(19) United States
(12) Patent Application Publication
(10) Pub. No.: US 2021/0147336 A1
(43) Pub. Date: May 20, 2021

(54) ORGANIC COMPOUND AND ORGANIC ELECTROLUMINESCENT DEVICE COMPRISING SAME

(71) Applicant: DOOSAN SOLUS CO., LTD., Iksan-si (KR)

(72) Inventors: Jae Yi SIM, Yongin-si (KR); Min-Sik EUM, Yongin-si (KR); Woo Jae PARK, Yongin-si (KR)

(73) Assignee: DOOSAN SOLUS CO., LTD., Iksan-si (KR)

(21) Appl. No.: 16/621,867

(22) PCT Filed: Nov. 21, 2017
(86) PCT No.: PCT/KR2017/013251

§ 371 Date: Dec. 12, 2019

(30) Foreign Application Priority Data
Jun. 14, 2017 (KR) 10-2017-0074873

(51) Int. Cl. C07C 211/61; H01L 51/00; C07D 307/91; C07D 333/76; C07D 209/82

(52) U.S. Cl. CPC C07C 211/61; H01L 51/0056; H01L 51/006; H01L 51/0072; H01L 51/0073; H01L 51/5064; C07D 307/91; C07D 333/76; C07D 209/82; H01L 51/0061; H01L 51/0074

(57) ABSTRACT

The present invention relates to a novel compound and an organic electroluminescent device including the same, and by using the compound according to the present invention in an organic material layer, preferably a light emitting layer, of an organic electroluminescent device, luminous efficiency, driving voltage, lifetime and the like of the organic electroluminescent device may be enhanced.

### TECHNICAL FIELD

This application claims priority to and the benefits of Korean Patent Application No. 10-2017-0074873, filed with the Korean Intellectual Property Office on Jun. 14, 2017, the entire contents of which are incorporated herein by reference.

The present invention relates to a novel organic compound capable of being used as a material for an organic electroluminescent device, and an organic electroluminescent device including the same.

### BACKGROUND ART

With the observation of organic thin film light emission made by Bemanose in 1950s as a start, studies on organic electroluminescent (EL) devices have been continued leading to blue electroluminescence using a single anthracene crystal in 1965, and in 1987, an organic electroluminescent device having a laminated structure divided into functional layers of a hole layer and a light emitting layer has been proposed by Tang. After that in order to manufacture organic electroluminescent devices with high efficiency and long lifetime, development has been made in the form of introducing each characteristic organic material layer into the device, which leads to the development of specialized materials used therein.

When a voltage is applied between the two electrodes in an organic electroluminescent device, holes and electrons are injected to an organic material layer from the anode and the cathode, respectively. When the injected holes and electrons meet, excitons are formed, and light emits when these excitons fall back to the ground state. Herein, materials used as the organic material layer may be divided into a light emitting material, a hole injection material, a hole transport material, an electron transport material, an electron injection material and the like depending on the function.

The light emitting material may be divided into, depending on the light emitting color, blue, green and red light emitting materials, and yellow and orange light emitting materials for obtaining better natural colors. In addition, in order to increase color purity and increase luminous efficiency through energy transfer, host/dopant series may be used as the light emitting material.

The dopant material may be divided into fluorescent dopants using organic materials and phosphorescent dopants using metal complex compounds including heavy atoms such as Ir or Pt.

Herein, development of phosphorescent materials may enhance luminous efficiency up to 4 times compared to fluorescence theoretically, and therefore, studies on phosphorescent host materials have been widely progressed as well as on phosphorescent dopants.

So far, NPB, BCP, Alq₃ and the like have been widely known as materials of a hole injection layer, a hole transport layer, a hole blocking layer and an electron transport layer, and anthracene derivatives have been reported as a material of alight emitting layer. Particularly, among light emitting layer materials, metal complex compounds including Ir such as Firpic, Ir(ppy)₃ or (acac)Ir(btp)₂ having advantages in terms of efficiency enhancement have been used as blue, green and red phosphorescent dopant materials, and 4,4-dicarbazolylbiphenyl (CBP) has been used as a phosphorescent host material.

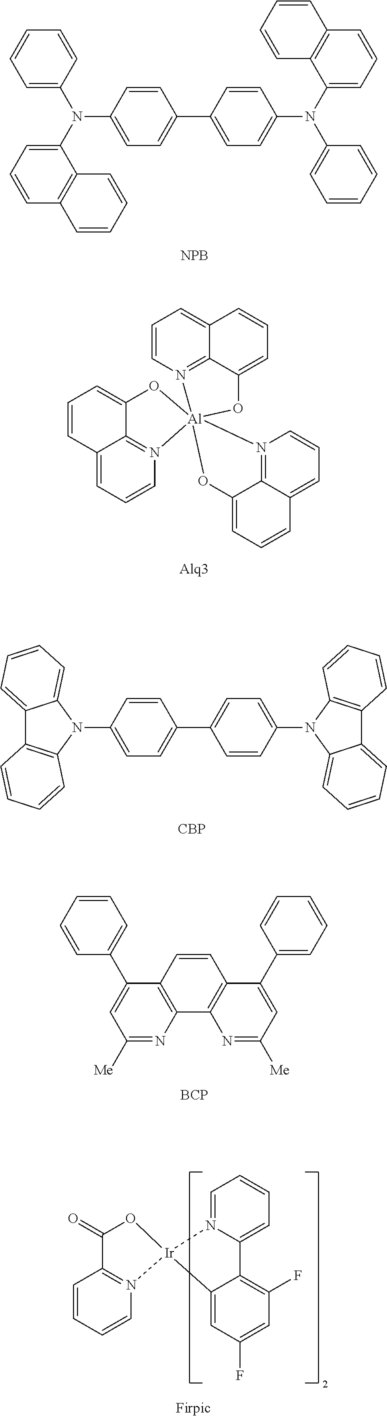

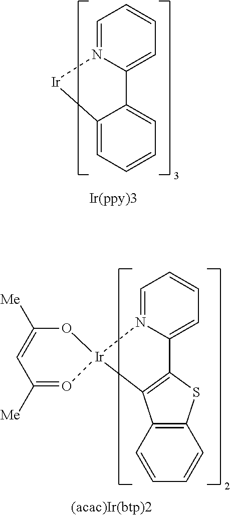

However, although being advantageous in terms of a light emission property, existing organic material layer materials have a low glass transition temperature and thereby have very unfavorable thermal stability, which is not satisfactory in terms of an organic electroluminescent device lifetime. Accordingly, development of organic material layer materials having superior performance has been required.

### DISCLOSURE

### Technical Problem

The present invention is directed to providing a novel organic compound capable of being used in an organic electroluminescent device, and having excellent hole and electron injection and transport abilities, a light emitting ability and the like.

The present invention is also directed to providing an organic electroluminescent device including the novel organic compound, and thereby exhibiting a low driving voltage, high luminous efficiency, and an enhanced lifetime.

### Technical Solution

In view of the above, one embodiment of the present invention provides a compound represented by the following Chemical Formula 1:

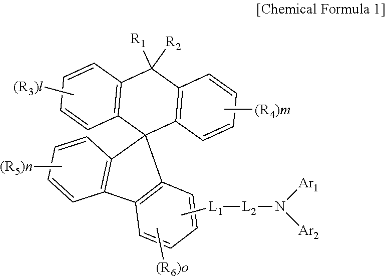

in Chemical Formula 1,

l, m and n are each independently an integer of 0 to 4;

o is an integer of 0 to 3;

R₁ and R₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group;

R₃ to R₆ are each independently selected from the group consisting of deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group, and when R₃ to R₆ are each present in plural numbers, these are the same as or different from each other;

the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of R₁ to R₆ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other;

L₁ and L₂ are each independently selected from the group consisting of a direct bond, a C₆˜C₁₈ arylene group and a heteroarylene group having 5 to 18 nuclear atoms;

Ar₁ and Ar₂ are each independently selected from the group consisting of hydrogen, deuterium, halogen, a cyano group, a nitro group, a C₁˜C₄₀ alkyl group, a C₂˜C₄ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group; and

the arylene group and the heteroarylene group of L₁ and L₂, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of Ar₁ and Ar₂ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₁˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other.

Another embodiment of the present invention provides an organic electroluminescent device including an anode, a cathode, and one or more organic material layers provided between the anode and the cathode, wherein at least one of the one or more organic material layers includes the compound of Chemical Formula 1.

The “halogen” in the present invention means fluorine, chlorine, bromine or iodine.

The “alkyl” in the present invention is a monovalent substituent derived from linear or branched saturated hydrocarbon having 1 to 40 carbon atoms. Examples thereof may include methyl, ethyl, propyl, isobutyl, sec-butyl, pentyl, iso-amyl, hexyl and the like, but are not limited thereto.

The “alkenyl” in the present invention is a monovalent substituent derived from linear or branched unsaturated hydrocarbon having one or more carbon-carbon double bonds and having 2 to 40 carbon atoms. Examples thereof may include vinyl, allyl, isopropenyl, 2-butenyl and the like, but are not limited thereto.

The “alkynyl” in the present invention is a monovalent substituent derived from linear or branched unsaturated hydrocarbon having one or more carbon-carbon triple bonds and having 2 to 40 carbon atoms. Examples thereof may include ethynyl, 2-propynyl and the like, but are not limited thereto.

The “aryl” in the present invention means a monovalent substituent derived from aromatic hydrocarbon having a single ring or two or more rings combined and having 6 to 60 carbon atoms. In addition, a monovalent substituent having two or more rings fused with each other, including only carbon (for example, the number of carbon atoms may be from 8 to 60) as a ring-forming atom, and with the whole molecule having non-aromaticity may also be included. Examples of such aryl may include phenyl, naphthyl, phenanthryl, anthryl, fluorenyl and the like, but are not limited thereto. The “heteroaryl” in the present invention means a monovalent substituent derived from monoheterocyclic or polyheterocyclic aromatic hydrocarbon having 5 to 60 nuclear atoms. Herein, one or more carbons, preferably 1 to 3 carbons, in the ring are substituted with a heteroatom selected from among N, O, P, S and Se. In addition, a monovalent group having two or more rings simply attached (pendant) or fused with each other, including a heteroatom selected from among N, O, P, S and Se as a ring-forming atom in addition to carbon, and with the whole molecule having non-aromaticity is interpreted to be included as well. Examples of such heteroaryl may include 6-membered monocyclic rings such as pyridyl, pyrazinyl, pyrimidinyl, pyridazinyl or triazinyl; polycyclic rings such as phenoxathienyl, indolizinyl, indolyl, purinyl, quinolyl, benzothiazole or carbazolyl; 2-furanyl, N-imidazolyl, 2-isoxazolyl, 2-pyridinyl, 2-pyrimidinyl and the like, but are not limited thereto.

The “aryloxy” in the present invention is a monovalent substituent represented by RO—, and R means aryl having 5 to 60 carbon atoms. Examples of such aryloxy may include phenyloxy, naphthyloxy, diphenyloxy and the like, but are not limited thereto.

The “alkyloxy” in the present invention is a monovalent substituent represented by R′O—, and R′ means alkyl having 1 to 40 carbon atoms and is interpreted to include a linear, branched or cyclic structure. Examples of such alkyloxy may include methoxy, ethoxy, n-propoxy, 1-propoxy, t-butoxy, n-butoxy, pentoxy and the like, but are not limited thereto.

The “arylamine” in the present invention means amine substituted with aryl having 6 to 60 carbon atoms.

The “cycloalkyl” in the present invention means a monovalent substituent derived from monocyclic or polycyclic non-aromatic hydrocarbon having 3 to 40 carbon atoms. Examples of such cycloalkyl may include cyclopropyl, cyclopentyl, cyclohexyl, norbomyl, adamantine and the like, but are not limited thereto.

The “heterocycloalkyl” in the present invention means a monovalent substituent derived from non-aromatic hydrocarbon having 3 to 40 nuclear atoms, and one or more carbons, preferably 1 to 3 carbons, in the ring are substituted with a heteroatom such as N, O, S or Se. Examples of such heterocycloalkyl may include morpholine, piperazine and the like, but are not limited thereto.

The “alkylsilyl” in the present invention means silyl substituted with alkyl having 1 to 40 carbon atoms, and the “arylsilyl” means silyl substituted with aryl having 5 to 60 carbon atoms.

The “fused ring” in the present invention means a fused aliphatic ring, a fused aromatic ring, a fused heteroaliphatic ring, a fused heteroaromatic ring, or a combined form thereof.

### Advantageous Effects

A compound of the present invention has excellent thermal stability, carrier transport ability, light emitting ability and the like, and therefore, is useful as a material of an organic material layer of an organic electroluminescent device.

In addition, an organic electroluminescent device including a compound of the present invention in an organic material layer has greatly enhanced properties in terms of light emitting performance, driving voltage, lifetime, efficiency and the like, and can be effectively used in a full color display panel and the like.

### DESCRIPTION OF DRAWINGS

FIG. 1 is a sectional diagram illustrating an organic electroluminescent device according to one embodiment of the present invention.

FIG. 2 is a sectional diagram illustrating an organic electroluminescent device according to one embodiment of the present invention.

10: Anode
20: Cathode
30: Organic Layer
31: Hole Transport Layer
32: Light Emitting Layer
33: Hole Transport Auxiliary Layer
34: Electron Transport Layer
35: Electron Transport Auxiliary Layer
36: Electron Injection Layer
37: Hole Injection Layer

### MODE FOR DISCLOSURE

One embodiment of the present invention provides a compound represented by the following Chemical Formula 1:

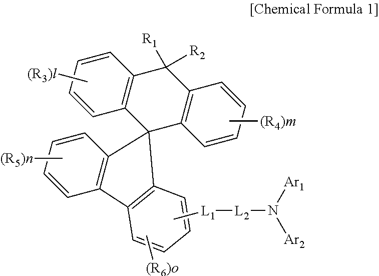

in Chemical Formula 1,

l, m and n are each independently an integer of 0 to 4;

o is an integer of 0 to 3;

R₁ and R₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group;

R₃ to R₆ are each independently selected from the group consisting of deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group, and when R₃ to R₆ are each present in plural numbers, these are the same as or different from each other;

the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of R₁ to R₆ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other;

L₁ and L₂ are each independently selected from the group consisting of a direct bond, a C₆˜C₁₈ arylene group and a heteroarylene group having 5 to 18 nuclear atoms;

Ar₁ and Ar₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group; and

the arylene group and the heteroarylene group of L₁ and L₂, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of Ar₁ and Ar₂ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other.

Hereinafter, the present invention will be described in detail.

1. Novel Organic Compound

A novel compound of the present invention may be represented by the following Chemical Formula 1:

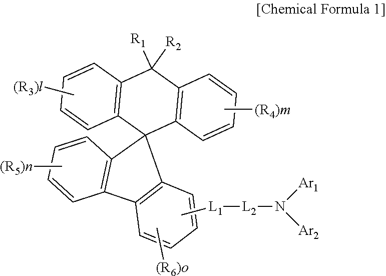

in Chemical Formula 1,

l, m and n are each independently an integer of 0 to 4;

o is an integer of 0 to 3;

R₁ and R₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group;

R₃ to R₆ are each independently selected from the group consisting of deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group, and when R₃ to R₆ are each present in plural numbers, these are the same as or different from each other;

the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of R₁ to R₆ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other;

L₁ and L₂ are each independently selected from the group consisting of a direct bond, a C₆˜C₁₈ arylene group and a heteroarylene group having 5 to 18 nuclear atoms;

Ar₁ and Ar₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group; and

the arylene group and the heteroarylene group of L₁ and L₂, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of Ar₁ and Ar₂ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other.

Spirodimethyl acridine structure-based materials have a very superior hole transport ability and show high hole mobility, and as a result, have a property of excellent luminous efficiency. In addition, thermal stability due to a high glass transition temperature, and a property of proper HOMO and LUMO energy levels between a hole injection layer and a light emitting layer are obtained enabling low voltage driving and thereby increasing a lifetime, and properties of amorphous crystallinity and high refractive index are effective in further increasing luminous efficiency.

Accordingly, the compound represented by Chemical Formula 1, a representative claimed structure of the present invention, has an excellent light emitting property, and may be used as a material of any one of a hole injection layer, a hole transport layer, a light emitting layer, an electron transport layer and an electron injection layer, organic materials of an organic electroluminescent device. Preferably, the compound represented by Chemical Formula 1 may be used as a material of a hole transport layer and a hole transport auxiliary layer.

According to preferred one embodiment of the present invention, the compound may be a compound represented by any one of the following Chemical Formulae 2 to 4:

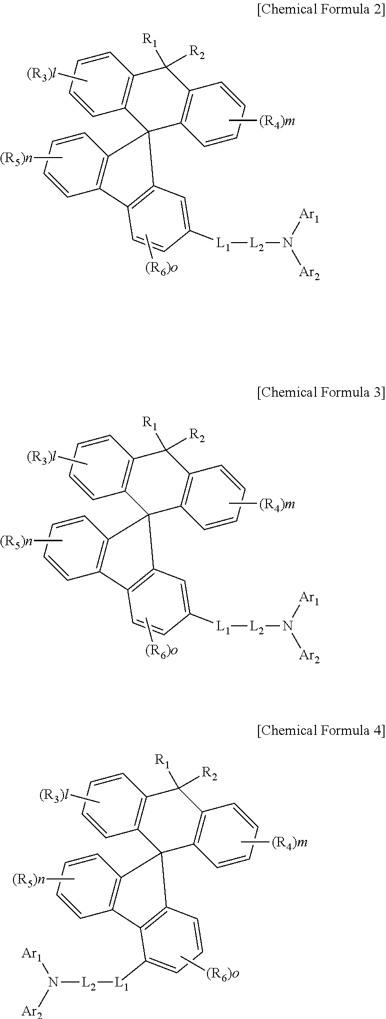

in Chemical Formulae 2 to 4,

R₁ to R₆, l, m, n, o, L₁, L₂, Ar₁ and Ar₂ have the same definitions as in Chemical Formula 1.

According to preferred one embodiment of the present invention, R₁ and R₂ are each independently selected from the group consisting of a C₁˜C₄₀ alkyl group, a C₆˜C₆₀ aryl group and a heteroaryl group having 5 to 60 nuclear atoms, and

the alkyl group, the aryl group and the heteroaryl group of R₁ and R₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, R₁ and R₂ are each independently selected from the group consisting of a methyl group, an ethyl group, a propyl group, a butyl group, a pentyl group, a phenyl group, a biphenyl group, a pyridinyl group, a pyrimidinyl group and a triazinyl group, and

the methyl group, the ethyl group, the propyl group, the butyl group, the pentyl group, the phenyl group, the biphenyl group, the pyridinyl group, the pyrimidinyl group and the triazinyl group of R₁ and R₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, R₁ and R₂ are each independently selected from the group consisting of a methyl group, an ethyl group, a propyl group, a butyl group, a pentyl group, a phenyl group, a biphenyl group, a pyridinyl group, a pyrimidinyl group and a triazinyl group, and

the methyl group, the ethyl group, the propyl group, the butyl group, the pentyl group, the phenyl group, the biphenyl group, the pyridinyl group, the pyrimidinyl group and the triazinyl group of R₁ and R₂ are each independently unsubstituted or substituted with one or more types of substituents selected from the group consisting of a methyl group, an ethyl group, a propyl group, a butyl group, a pentyl group, a phenyl group, a biphenyl group, a pyridinyl group, a pyrimidinyl group and a triazinyl group, and when substituted with a plurality of the substituents, these are the same as or different from each other.

According to preferred one embodiment of the present invention, L₁ and L₂ may be each independently a direct bond, or a linker selected from the group consisting of the following Chemical Formulae A-1 to A-4, and may be more preferably a direct bond, or a linker represented by A-1 or A-2:

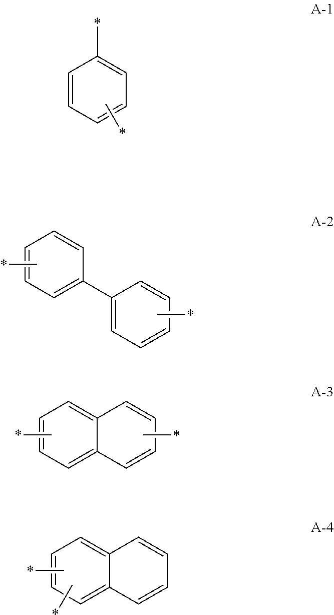

in Chemical Formulae A-1 to A-4,

* means a part where a bond is formed.

According to preferred one embodiment of the present invention, Ar₁ and Ar₂ are selected from the group consisting of a C₁˜C₄₀ alkyl group, a C₆˜C₆₀ aryl group and a heteroaryl group having 5 to 60 nuclear atoms, and

the alkyl group, the aryl group and the heteroaryl group of Ar₁ and Ar₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, Ar₁ and Ar₂ are selected from the group consisting of a methyl group, an ethyl group, a propyl group, a butyl group, a pentyl group, a phenyl group, a biphenyl group, a fluorenyl group, a carbazolyl group, a dibenzofuranyl group, a dibenzothiophenyl group, a pyridinyl group, a pyrimidinyl group, a triazinyl group, a naphthalenyl group, a triazolopyridinyl group, a quinolinyl group, an isoquinolinyl group, a cinnolinyl group, a quinoxalinyl group and a quinazolinyl group, and

the methyl group, the ethyl group, the propyl group, the butyl group, the pentyl group, the phenyl group, the biphenyl group, the fluorenyl group, the carbazolyl group, the dibenzofuranyl group, the dibenzothiophenyl group, the pyridinyl group, the pyrimidinyl group, the triazinyl group, the naphthalenyl group, the triazolopyridinyl group, the quinolinyl group, the isoquinolinyl group, the cinnolinyl group, the quinoxalinyl group and the quinazolinyl group of Ar₁ and Ar₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, Ar₁ and Ar₂ are selected from the group consisting of a methyl group, an ethyl group, a propyl group, a butyl group, a pentyl group, a phenyl group, a biphenyl group, a fluorenyl group, a carbazolyl group, a dibenzofuranyl group, a dibenzothiophenyl group, a pyridinyl group, a pyrimidinyl group, a triazinyl group, a naphthalenyl group, a triazolopyridinyl group, a quinolinyl group, an isoquinolinyl group, a cinnolinyl group, a quinoxalinyl group and a quinazolinyl group, and

the methyl group, the ethyl group, the propyl group, the butyl group, the pentyl group, the phenyl group, the biphenyl group, the fluorenyl group, the carbazolyl group, the dibenzofuranyl group, the dibenzothiophenyl group, the pyridinyl group, the pyrimidinyl group, the triazinyl group, the naphthalenyl group, the triazolopyridinyl group, the quinolinyl group, the isoquinolinyl group, the cinnolinyl group, the quinoxalinyl group and the quinazolinyl group of Ar₁ and Ar₂ are each independently unsubstituted or substituted with one or more types of substituents selected from the group consisting of a methyl group, an ethyl group, a propyl group, a butyl group, a pentyl group, a phenyl group, a biphenyl group, a fluorenyl group, a carbazolyl group, a dibenzofuranyl group, a dibenzothiophenyl group, a pyridinyl group, a pyrimidinyl group, a triazinyl group, a naphthalenyl group, a triazolopyridinyl group, a quinolinyl group, an isoquinolinyl group, a cinnolinyl group, a quinoxalinyl group and a quinazolinyl group, and when substituted with a plurality of the substituents, these are the same as or different from each other.

According to preferred one embodiment of the present invention, Ar₁ and Ar₂ may be a substituent represented by the following Chemical Formula 5 or 6:

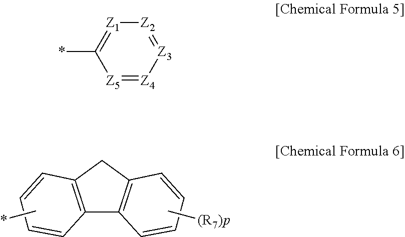

in Chemical Formulae 5 and 6,

* means a part where a bond is formed;

p is an integer of 0 to 4;

Z₁ to Z₅ are each independently N or C(R₈);

X₁ is O, S, N(R₉) or C(R₁₀)(R₁₁);

R₇ is selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₃˜C₄₀ cycloalkyl group, a heterocycloalkyl group having 3 to 40 nuclear atoms, a C₆˜C₆₀ arylamine group, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, or may bond to adjacent groups to form a fused ring, and when R₇ is present in plural numbers, these are the same as or different from each other;

R₈ to R₁₁ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₃˜C₄₀ cycloalkyl group, a heterocycloalkyl group having 3 to 40 nuclear atoms, a C₆˜C₆₀ arylamine group, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, or may bond to adjacent groups to form a fused ring, and when R₈ is present in plural numbers, these are the same as or different from each other; and

the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the mono or diarylphosphinyl group and the arylsilyl group of R₇ to R₁₁ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, and when substituted with a plurality of the substituents, these are the same as or different from each other.

According to preferred one embodiment of the present invention, R₈ to R₁₁ are each independently selected from the group consisting of a C₁˜C₄₀ alkyl group, a C₆˜C₆₀ aryl group and a heteroaryl group having 5 to 60 nuclear atoms, and

the alkyl group, the aryl group and the heteroaryl group of R₈ to R₁₁ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, R₈ to R₁₁ are each independently selected from the group consisting of a methyl group, an ethyl group, a propyl group, a butyl group, a pentyl group, a phenyl group, a biphenyl group, a pyridinyl group, a pyrimidinyl group and a triazinyl group, and

the methyl group, the ethyl group, the propyl group, the butyl group, the pentyl group, the phenyl group, the biphenyl group, the pyridinyl group, the pyrimidinyl group and the triazinyl group of R₈ to R₁₁ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

According to preferred one embodiment of the present invention, R₈ to R₁₁ are each independently selected from the group consisting of a methyl group, an ethyl group, a propyl group, a butyl group, a pentyl group, a phenyl group, a biphenyl group, a pyridinyl group, a pyrimidinyl group and a triazinyl group, and

the methyl group, the ethyl group, the propyl group, the butyl group, the pentyl group, the phenyl group, the biphenyl group, the pyridinyl group, the pyrimidinyl group and the triazinyl group of R₈ to R₁₁ are each independently unsubstituted or substituted with one or more types of substituents selected from the group consisting of a methyl group, an ethyl group, a propyl group, a butyl group, a pentyl group, a phenyl group, a biphenyl group, a pyridinyl group, a pyrimidinyl group and a triazinyl group, and when substituted with a plurality of the substituents, these are the same as or different from each other.

According to preferred one embodiment of the present invention, the compound may be N-([1,1′-biphenyl]-4-yl)-N-(4-(10,10-dimethyl-10H-spiro[anthracene-9,9′-fluoren]-2′-yl)phenyl)-[1,1′-biphenyl]-4-amine or N-(4-(10,10-dimethyl-10H-spiro[anthracene-9,9′-fluoren]-2′-yl)phenyl)-N-phenyldibenzo[b,d]furan-2-amine.

The compound represented by Chemical Formula 1 of the present invention may be represented by the following compounds, but is not limited thereto:

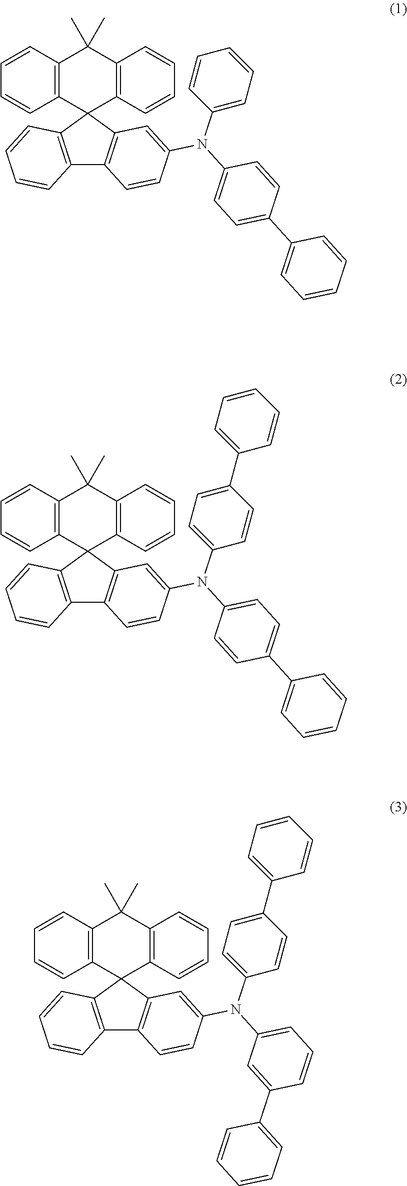 [SMILES: CC1(C)c2ccccc2C2(c3ccccc3-c3ccc(N(c4ccccc4)c4ccc(-c5ccccc5)cc4)cc32)c2ccccc21 ; CC1(C)c2ccccc2C2(c3ccccc3-c3ccc(N(c4ccc(-c5ccccc5)cc4)c4ccc(-c5ccccc5)cc4)cc32)c2ccccc21 ; CC1(C)c2ccccc2C2(c3ccccc3-c3ccc(N(c4ccc(-c5ccccc5)cc4)c4cccc(-c5ccccc5)c4)cc32)c2ccccc21]

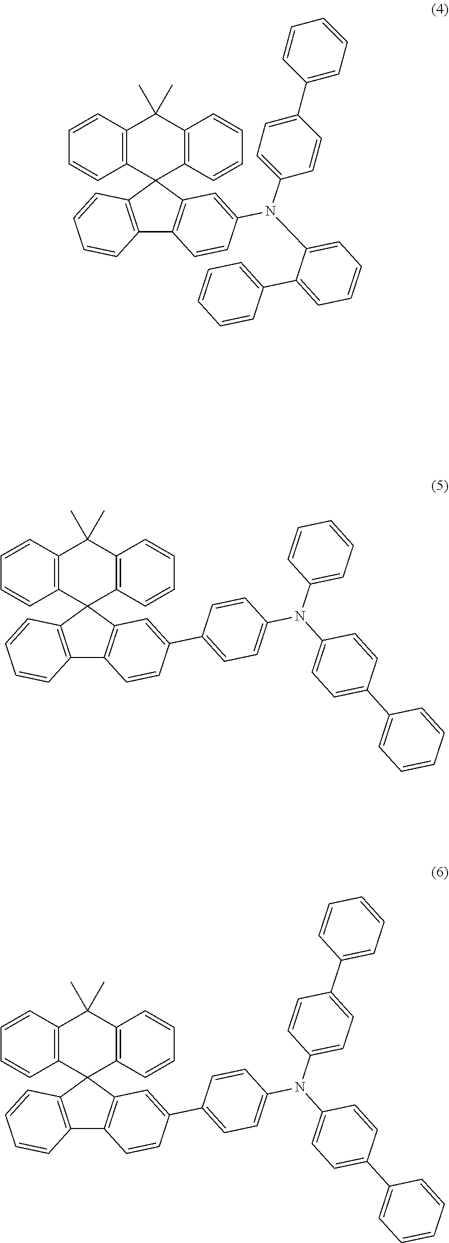 [SMILES: CC1(C)c2ccccc2C2(c3ccccc3-c3ccc(N(c4ccc(-c5ccccc5)cc4)c4ccccc4-c4ccccc4)cc32)c2ccccc21 ; CC1(C)c2ccccc2C2(c3ccccc3-c3ccc(-c4ccc(N(c5ccccc5)c5ccc(-c6ccccc6)cc5)cc4)cc32)c2ccccc21 ; CC1(C)c2ccccc2C2(c3ccccc3-c3ccc(-c4ccc(N(c5ccc(-c6ccccc6)cc5)c5ccc(-c6ccccc6)cc5)cc4)cc32)c2ccccc21]

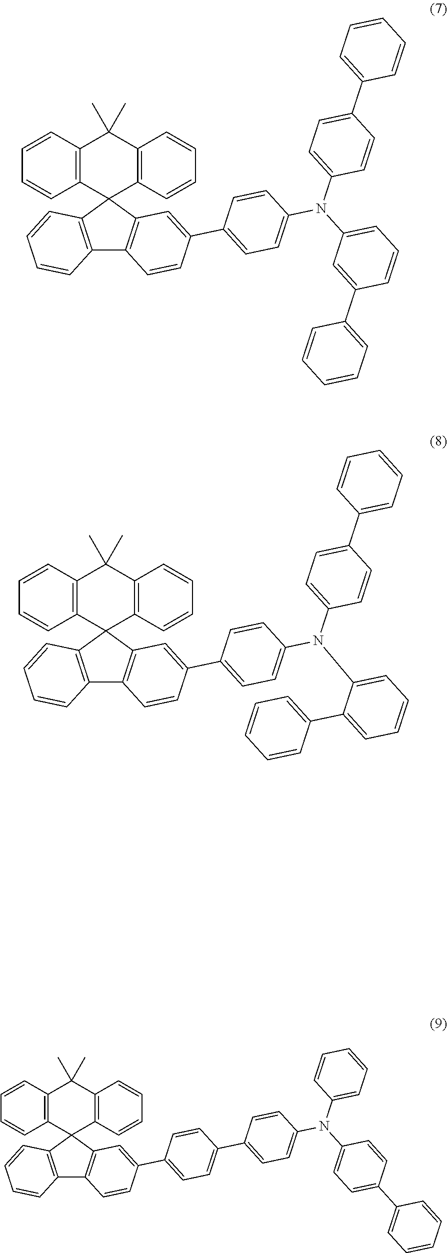 [SMILES: CC1(C)c2ccccc2C2(c3ccccc3-c3ccc(-c4ccc(N(c5ccc(-c6ccccc6)cc5)c5cccc(-c6ccccc6)c5)cc4)cc32)c2ccccc21 ; CC1(C)c2ccccc2C2(c3ccccc3-c3ccc(-c4ccc(N(c5ccc(-c6ccccc6)cc5)c5ccccc5-c5ccccc5)cc4)cc32)c2ccccc21 ; CC1(C)c2ccccc2C2(c3ccccc3-c3ccc(-c4ccc(-c5ccc(N(c6ccccc6)c6ccc(-c7ccccc7)cc6)cc5)cc4)cc32)c2ccccc21]

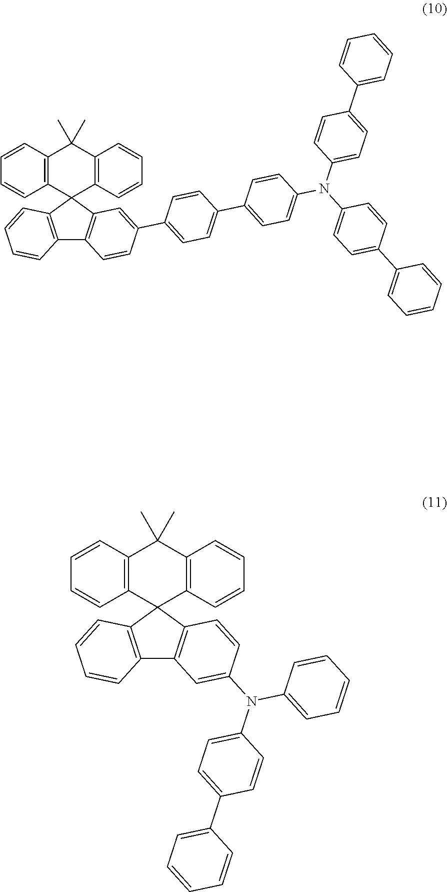

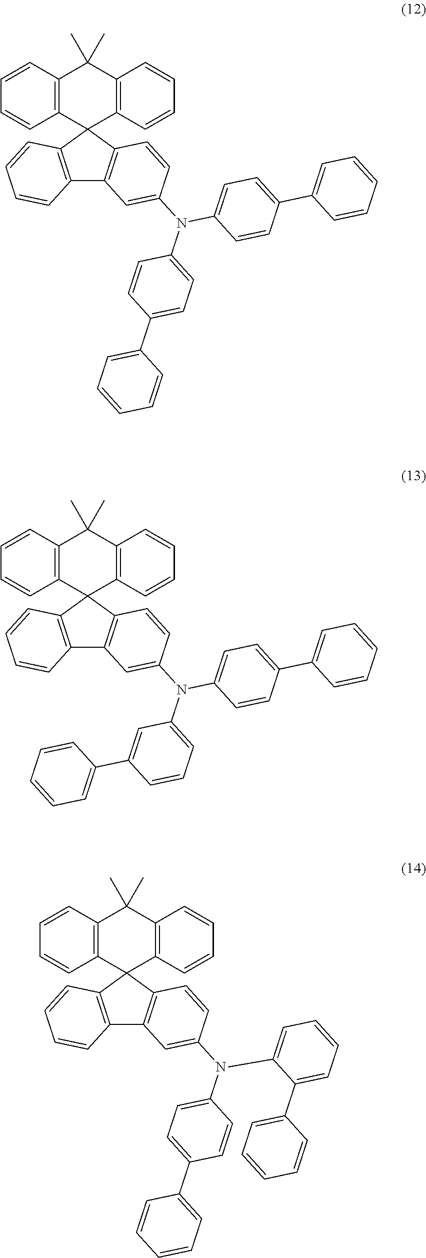 [SMILES: CC1(C)c2ccccc2C2(c3ccccc3-c3cc(N(c4ccc(-c5ccccc5)cc4)c4ccc(-c5ccccc5)cc4)ccc32)c2ccccc21 ; CC1(C)c2ccccc2C2(c3ccccc3-c3cc(N(c4ccc(-c5ccccc5)cc4)c4cccc(-c5ccccc5)c4)ccc32)c2ccccc21 ; CC1(C)c2ccccc2C2(c3ccccc3-c3cc(N(c4ccc(-c5ccccc5)cc4)c4ccccc4-c4ccccc4)ccc32)c2ccccc21]

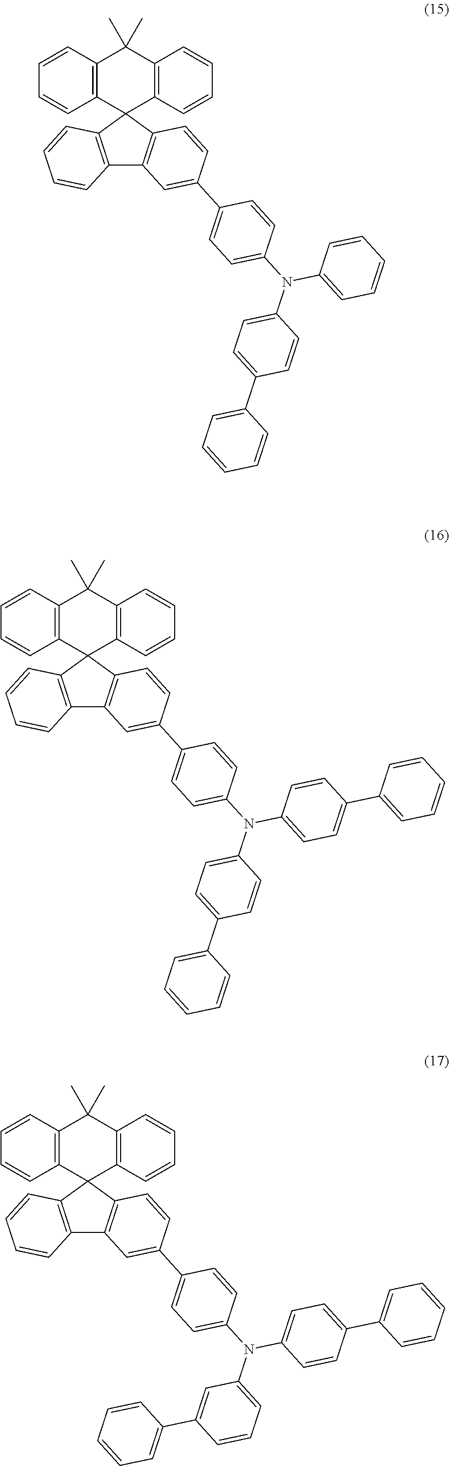 [SMILES: CC1(C)c2ccccc2C2(c3ccccc3-c3cc(-c4ccc(N(c5ccccc5)c5ccc(-c6ccccc6)cc5)cc4)ccc32)c2ccccc21 ; CC1(C)c2ccccc2C2(c3ccccc3-c3cc(-c4ccc(N(c5ccc(-c6ccccc6)cc5)c5ccc(-c6ccccc6)cc5)cc4)ccc32)c2ccccc21 ; CC1(C)c2ccccc2C2(c3ccccc3-c3cc(-c4ccc(N(c5ccc(-c6ccccc6)cc5)c5cccc(-c6ccccc6)c5)cc4)ccc32)c2ccccc21]

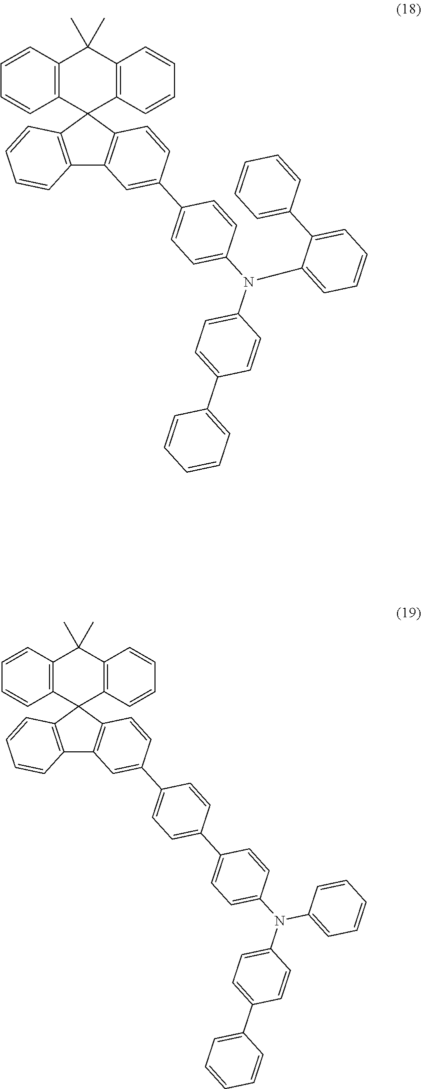 [SMILES: CC1(C)c2ccccc2C2(c3ccccc3-c3cc(-c4ccc(N(c5ccc(-c6ccccc6)cc5)c5ccccc5-c5ccccc5)cc4)ccc32)c2ccccc21 ; CC1(C)c2ccccc2C2(c3ccccc3-c3cc(-c4ccc(-c5ccc(N(c6ccccc6)c6ccc(-c7ccccc7)cc6)cc5)cc4)ccc32)c2ccccc21]

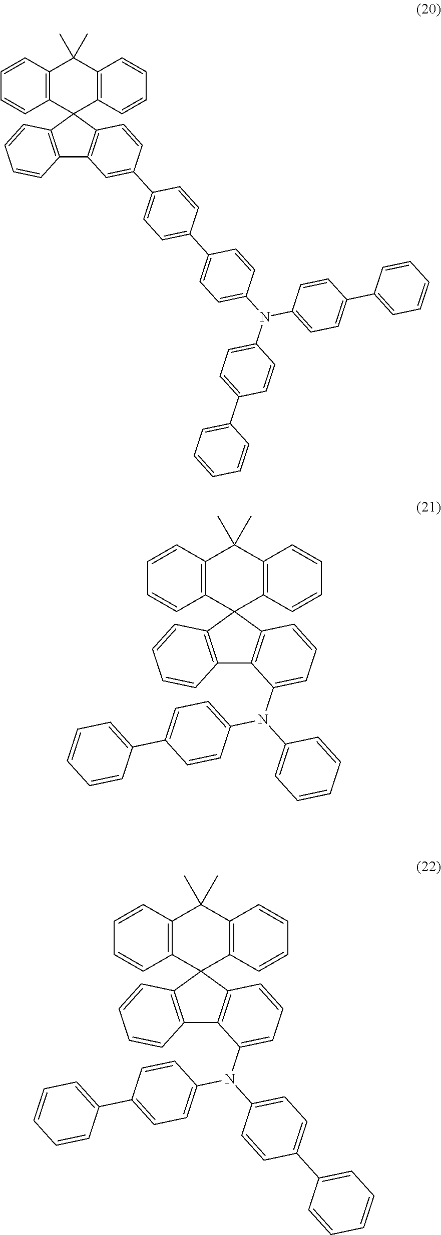

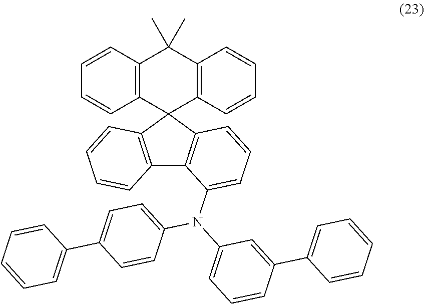 [SMILES: CC1(C)c2ccccc2C2(c3ccccc3-c3c(N(c4ccc(-c5ccccc5)cc4)c4cccc(-c5ccccc5)c4)cccc32)c2ccccc21]

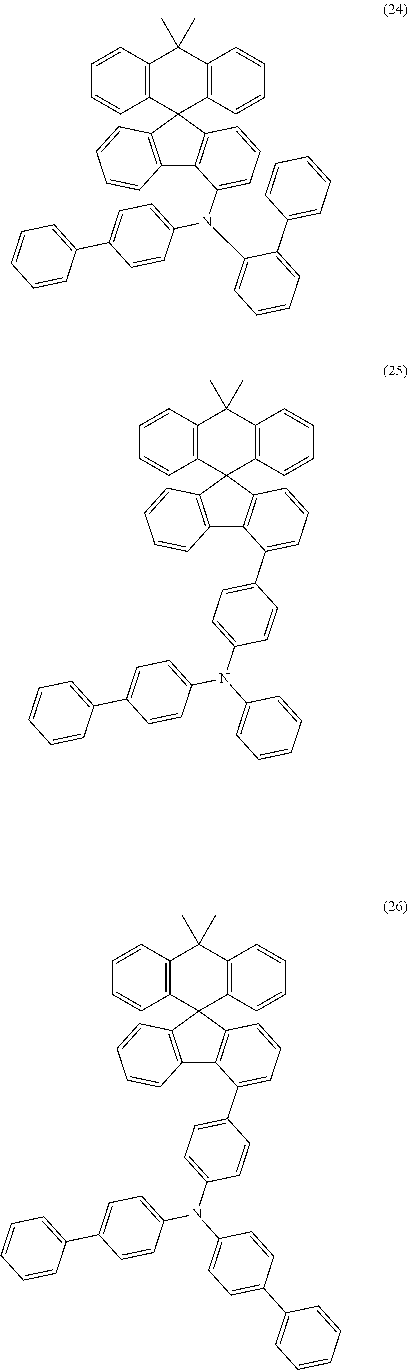 [SMILES: CC1(C)c2ccccc2C2(c3ccccc3-c3c(N(c4ccc(-c5ccccc5)cc4)c4ccccc4-c4ccccc4)cccc32)c2ccccc21 ; CC1(C)c2ccccc2C2(c3ccccc3-c3c(-c4ccc(N(c5ccccc5)c5ccc(-c6ccccc6)cc5)cc4)cccc32)c2ccccc21 ; CC1(C)c2ccccc2C2(c3ccccc3-c3c(-c4ccc(N(c5ccc(-c6ccccc6)cc5)c5ccc(-c6ccccc6)cc5)cc4)cccc32)c2ccccc21]

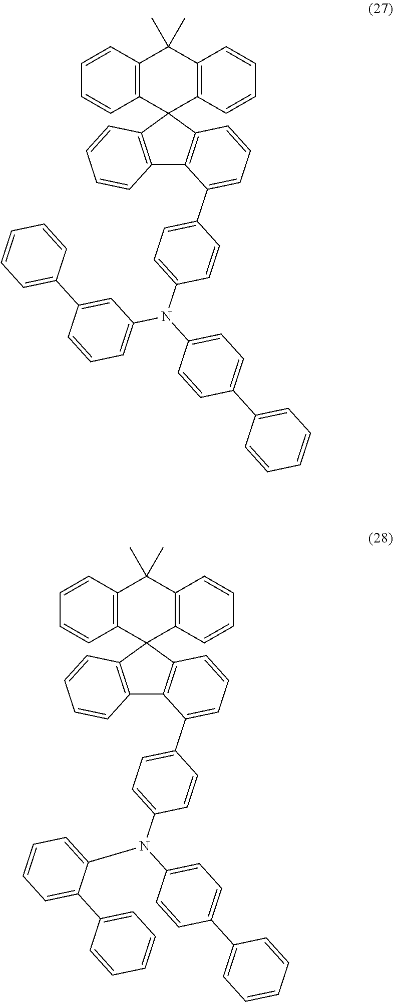 [SMILES: CC1(C)c2ccccc2C2(c3ccccc3-c3c(-c4ccc(N(c5ccc(-c6ccccc6)cc5)c5cccc(-c6ccccc6)c5)cc4)cccc32)c2ccccc21 ; CC1(C)c2ccccc2C2(c3ccccc3-c3c(-c4ccc(N(c5ccc(-c6ccccc6)cc5)c5ccccc5-c5ccccc5)cc4)cccc32)c2ccccc21]

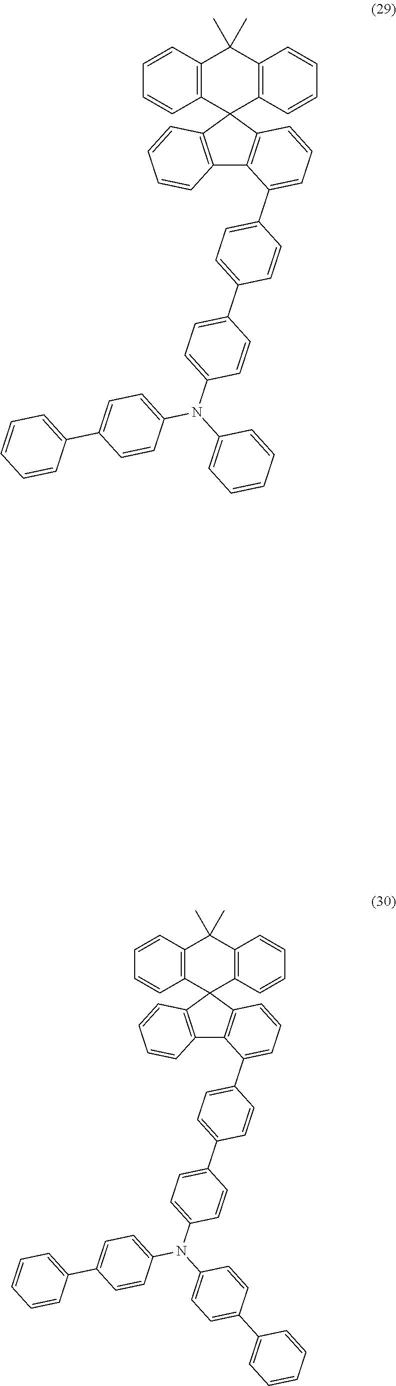 [SMILES: CC1(C)c2ccccc2C2(c3ccccc3-c3c(-c4ccc(-c5ccc(N(c6ccccc6)c6ccc(-c7ccccc7)cc6)cc5)cc4)cccc32)c2ccccc21 ; CC1(C)c2ccccc2C2(c3ccccc3-c3c(-c4ccc(-c5ccc(N(c6ccc(-c7ccccc7)cc6)c6ccc(-c7ccccc7)cc6)cc5)cc4)cccc32)c2ccccc21]

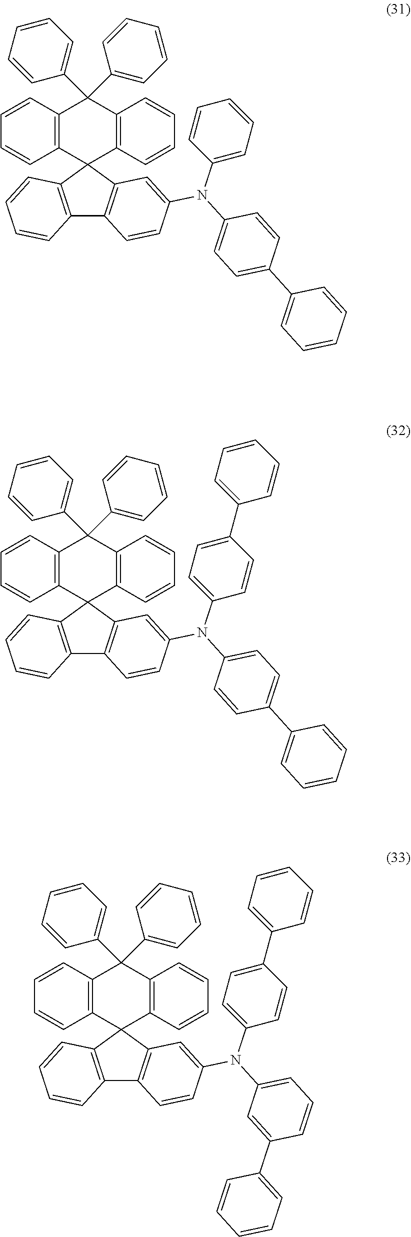 [SMILES: c1ccc(-c2ccc(N(c3ccccc3)c3ccc4c(c3)C3(c5ccccc5-4)c4ccccc4C(c4ccccc4)(c4ccccc4)c4ccccc43)cc2)cc1 ; c1ccc(-c2ccc(N(c3ccc(-c4ccccc4)cc3)c3ccc4c(c3)C3(c5ccccc5-4)c4ccccc4C(c4ccccc4)(c4ccccc4)c4ccccc43)cc2)cc1 ; c1ccc(-c2ccc(N(c3cccc(-c4ccccc4)c3)c3ccc4c(c3)C3(c5ccccc5-4)c4ccccc4C(c4ccccc4)(c4ccccc4)c4ccccc43)cc2)cc1]

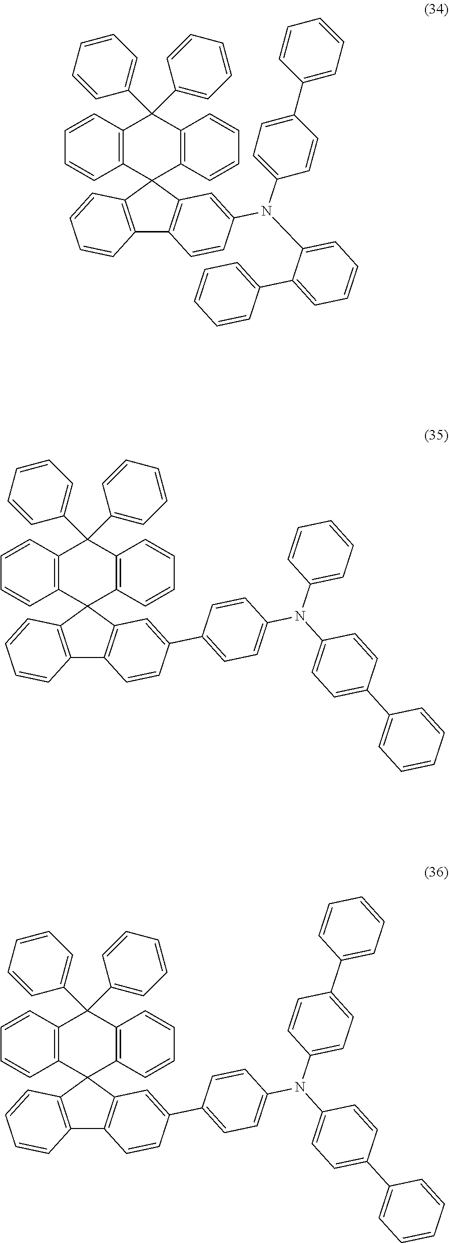 [SMILES: c1ccc(-c2ccc(N(c3ccc4c(c3)C3(c5ccccc5-4)c4ccccc4C(c4ccccc4)(c4ccccc4)c4ccccc43)c3ccccc3-c3ccccc3)cc2)cc1 ; c1ccc(-c2ccc(N(c3ccccc3)c3ccc(-c4ccc5c(c4)C4(c6ccccc6-5)c5ccccc5C(c5ccccc5)(c5ccccc5)c5ccccc54)cc3)cc2)cc1 ; c1ccc(-c2ccc(N(c3ccc(-c4ccccc4)cc3)c3ccc(-c4ccc5c(c4)C4(c6ccccc6-5)c5ccccc5C(c5ccccc5)(c5ccccc5)c5ccccc54)cc3)cc2)cc1]

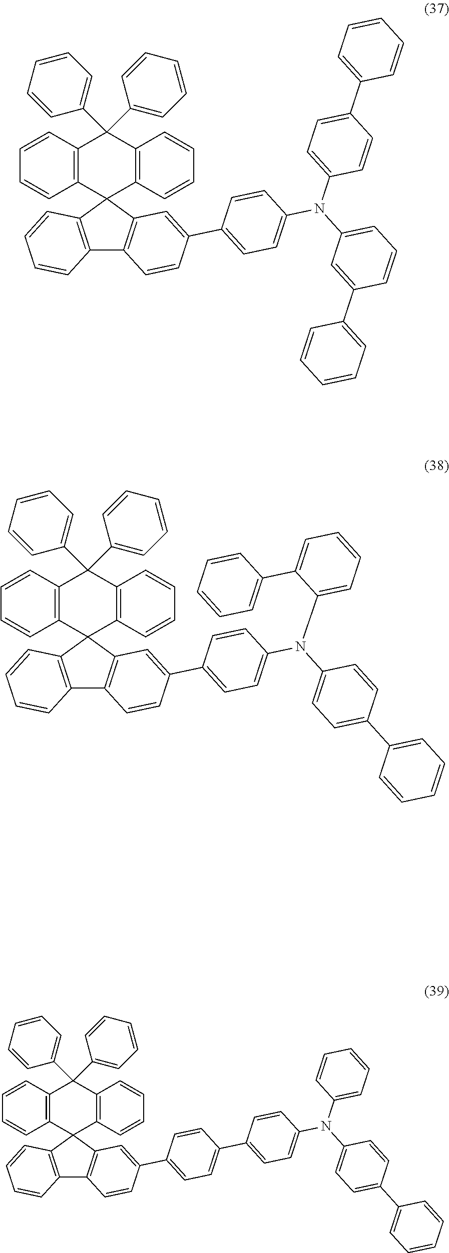 [SMILES: c1ccc(-c2ccc(N(c3ccc(-c4ccc5c(c4)C4(c6ccccc6-5)c5ccccc5C(c5ccccc5)(c5ccccc5)c5ccccc54)cc3)c3cccc(-c4ccccc4)c3)cc2)cc1 ; c1ccc(-c2ccc(N(c3ccc(-c4ccc5c(c4)C4(c6ccccc6-5)c5ccccc5C(c5ccccc5)(c5ccccc5)c5ccccc54)cc3)c3ccccc3-c3ccccc3)cc2)cc1 ; c1ccc(-c2ccc(N(c3ccccc3)c3ccc(-c4ccc(-c5ccc6c(c5)C5(c7ccccc7-6)c6ccccc6C(c6ccccc6)(c6ccccc6)c6ccccc65)cc4)cc3)cc2)cc1]

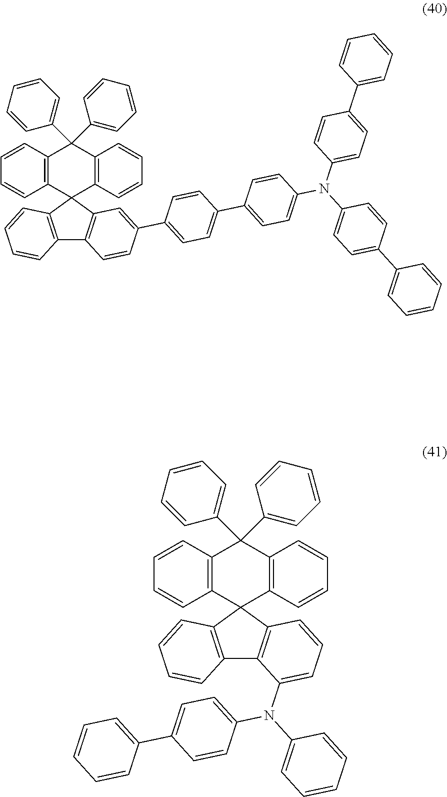

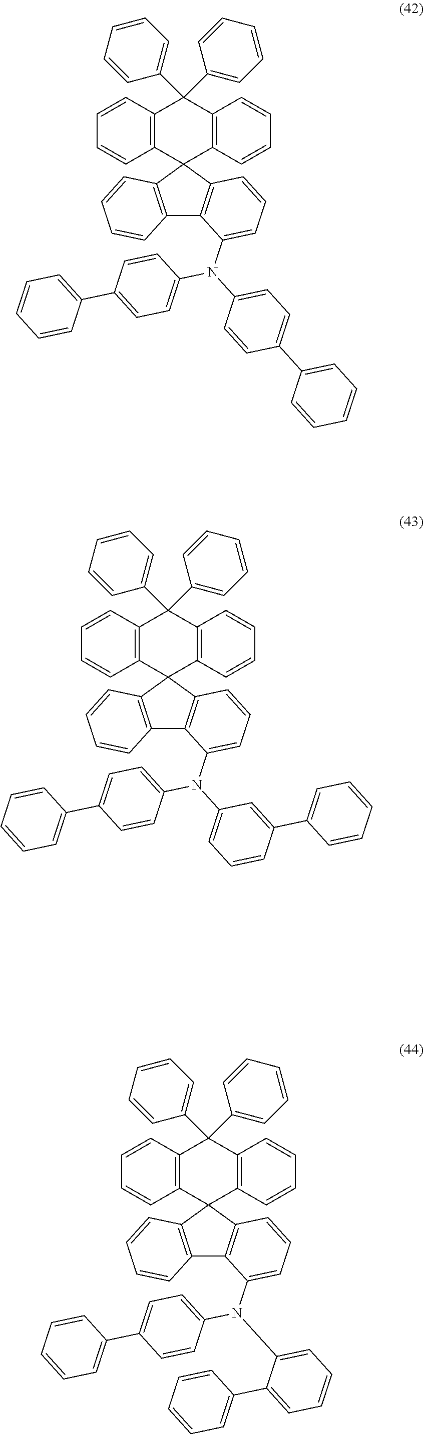 [SMILES: c1ccc(-c2ccc(N(c3ccc(-c4ccccc4)cc3)c3cccc4c3-c3ccccc3C43c4ccccc4C(c4ccccc4)(c4ccccc4)c4ccccc43)cc2)cc1 ; c1ccc(-c2ccc(N(c3cccc(-c4ccccc4)c3)c3cccc4c3-c3ccccc3C43c4ccccc4C(c4ccccc4)(c4ccccc4)c4ccccc43)cc2)cc1 ; c1ccc(-c2ccc(N(c3ccccc3-c3ccccc3)c3cccc4c3-c3ccccc3C43c4ccccc4C(c4ccccc4)(c4ccccc4)c4ccccc43)cc2)cc1]

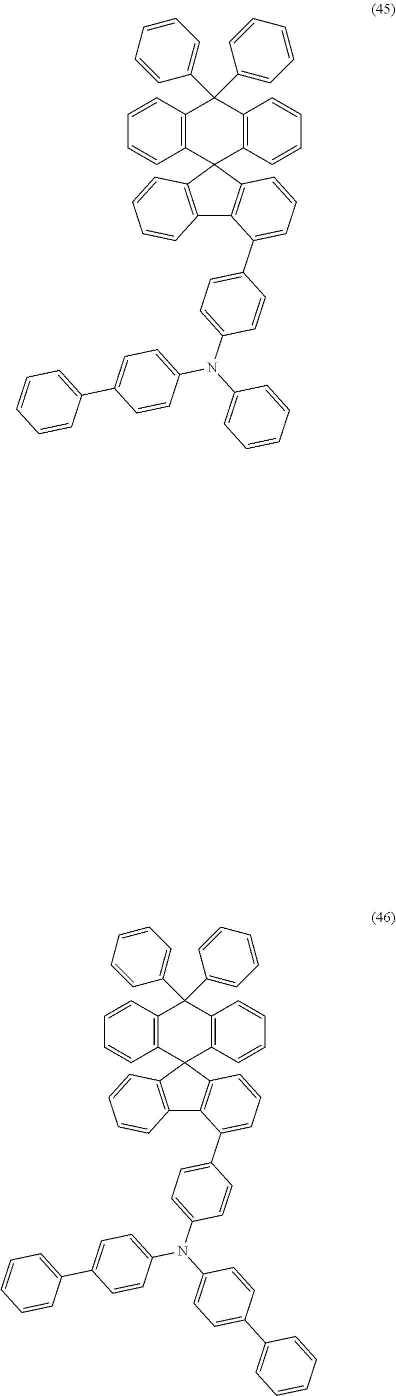 [SMILES: c1ccc(-c2ccc(N(c3ccccc3)c3ccc(-c4cccc5c4-c4ccccc4C54c5ccccc5C(c5ccccc5)(c5ccccc5)c5ccccc54)cc3)cc2)cc1 ; c1ccc(-c2ccc(N(c3ccc(-c4ccccc4)cc3)c3ccc(-c4cccc5c4-c4ccccc4C54c5ccccc5C(c5ccccc5)(c5ccccc5)c5ccccc54)cc3)cc2)cc1]

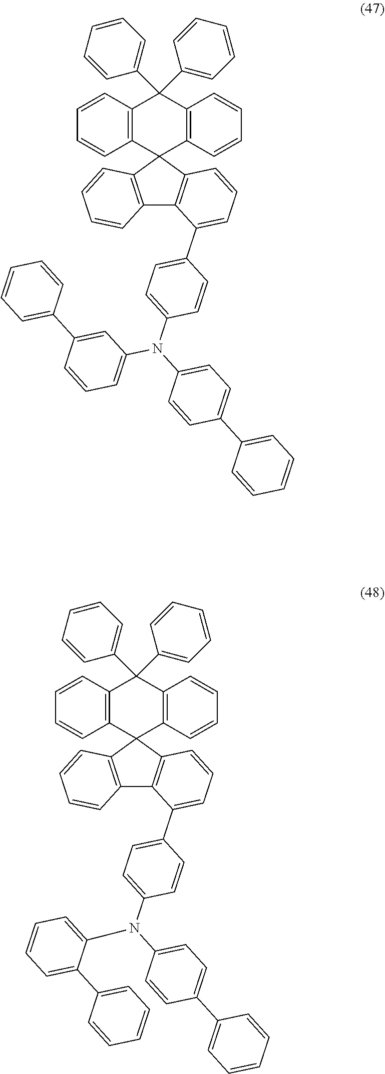 [SMILES: c1ccc(-c2ccc(N(c3ccc(-c4cccc5c4-c4ccccc4C54c5ccccc5C(c5ccccc5)(c5ccccc5)c5ccccc54)cc3)c3cccc(-c4ccccc4)c3)cc2)cc1 ; c1ccc(-c2ccc(N(c3ccc(-c4cccc5c4-c4ccccc4C54c5ccccc5C(c5ccccc5)(c5ccccc5)c5ccccc54)cc3)c3ccccc3-c3ccccc3)cc2)cc1]

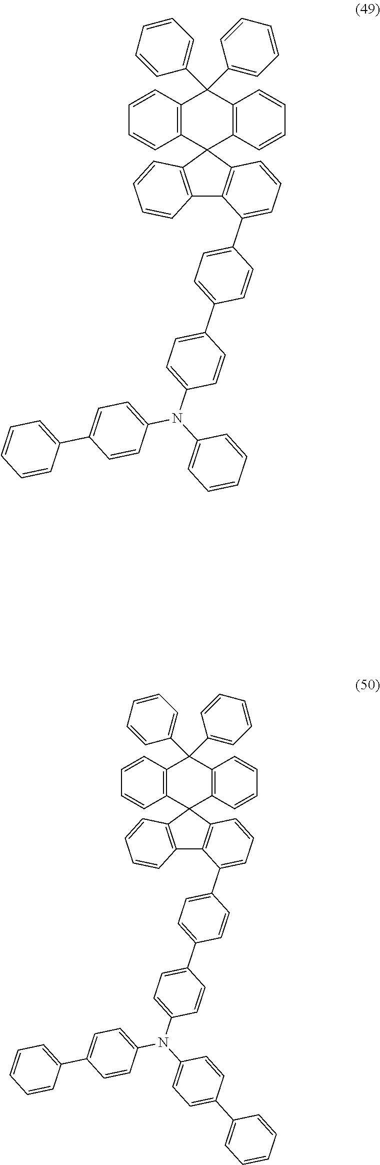 [SMILES: c1ccc(-c2ccc(N(c3ccccc3)c3ccc(-c4ccc(-c5cccc6c5-c5ccccc5C65c6ccccc6C(c6ccccc6)(c6ccccc6)c6ccccc65)cc4)cc3)cc2)cc1 ; c1ccc(-c2ccc(N(c3ccc(-c4ccccc4)cc3)c3ccc(-c4ccc(-c5cccc6c5-c5ccccc5C65c6ccccc6C(c6ccccc6)(c6ccccc6)c6ccccc65)cc4)cc3)cc2)cc1]

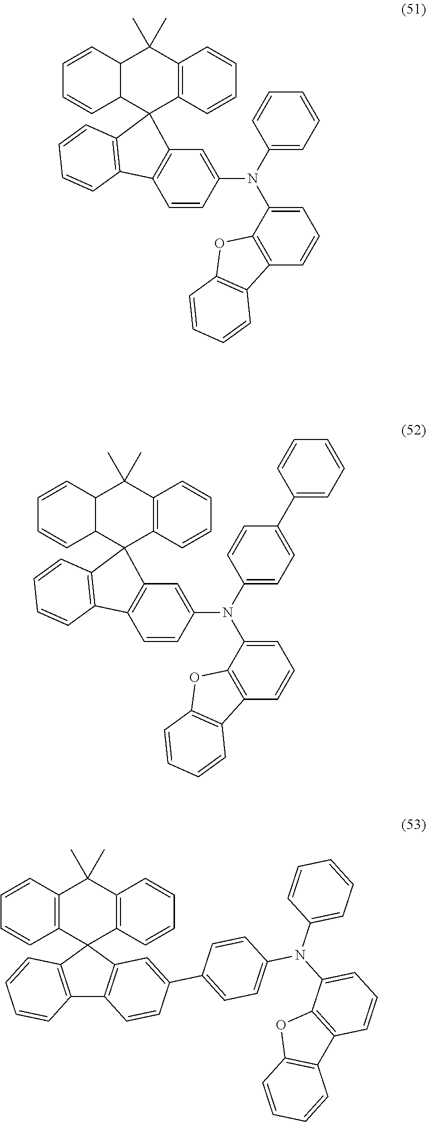 [SMILES: CC1(C)c2ccccc2C2(c3ccccc3-c3ccc(N(c4ccccc4)c4cccc5c4oc4ccccc45)cc32)C2C=CC=CC21 ; CC1(C)c2ccccc2C2(c3ccccc3-c3ccc(N(c4ccc(-c5ccccc5)cc4)c4cccc5c4oc4ccccc45)cc32)C2C=CC=CC21 ; CC1(C)c2ccccc2C2(c3ccccc3-c3ccc(-c4ccc(N(c5ccccc5)c5cccc6c5oc5ccccc56)cc4)cc32)c2ccccc21]

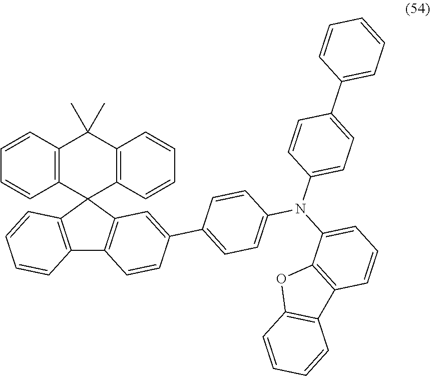 [SMILES: CC1(C)c2ccccc2C2(c3ccccc3-c3ccc(-c4ccc(N(c5ccc(-c6ccccc6)cc5)c5cccc6c5oc5ccccc56)cc4)cc32)c2ccccc21]

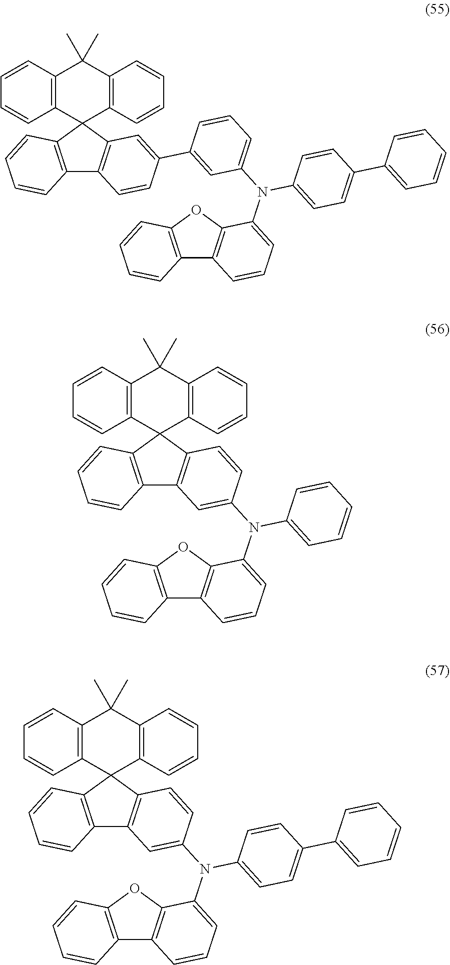 [SMILES: CC1(C)c2ccccc2C2(c3ccccc3-c3ccc(-c4cccc(N(c5ccc(-c6ccccc6)cc5)c5cccc6c5oc5ccccc56)c4)cc32)c2ccccc21 ; CC1(C)c2ccccc2C2(c3ccccc3-c3cc(N(c4ccccc4)c4cccc5c4oc4ccccc45)ccc32)c2ccccc21 ; CC1(C)c2ccccc2C2(c3ccccc3-c3cc(N(c4ccc(-c5ccccc5)cc4)c4cccc5c4oc4ccccc45)ccc32)c2ccccc21]

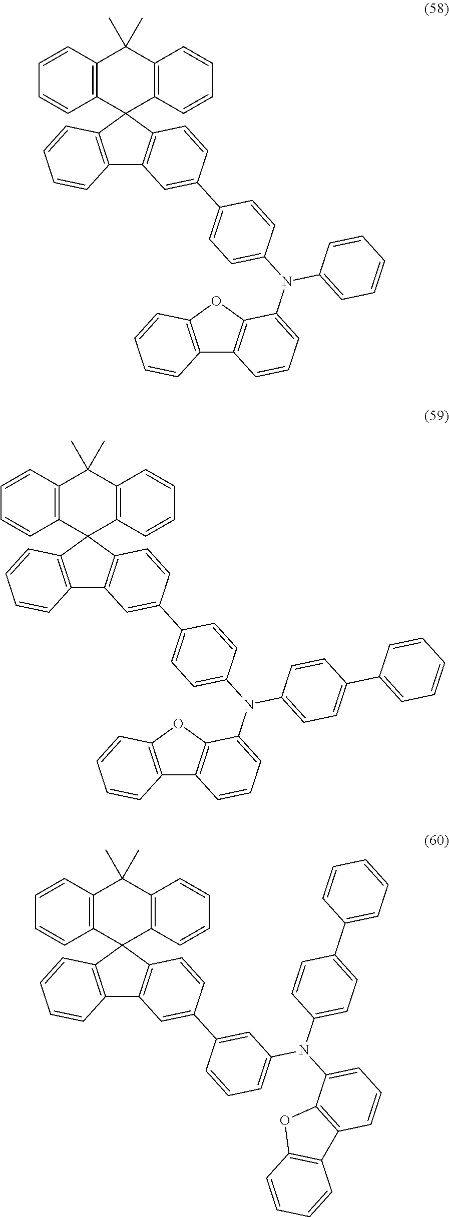 [SMILES: CC1(C)c2ccccc2C2(c3ccccc3-c3cc(-c4ccc(N(c5ccccc5)c5cccc6c5oc5ccccc56)cc4)ccc32)c2ccccc21 ; CC1(C)c2ccccc2C2(c3ccccc3-c3cc(-c4ccc(N(c5ccc(-c6ccccc6)cc5)c5cccc6c5oc5ccccc56)cc4)ccc32)c2ccccc21 ; CC1(C)c2ccccc2C2(c3ccccc3-c3cc(-c4cccc(N(c5ccc(-c6ccccc6)cc5)c5cccc6c5oc5ccccc56)c4)ccc32)c2ccccc21]

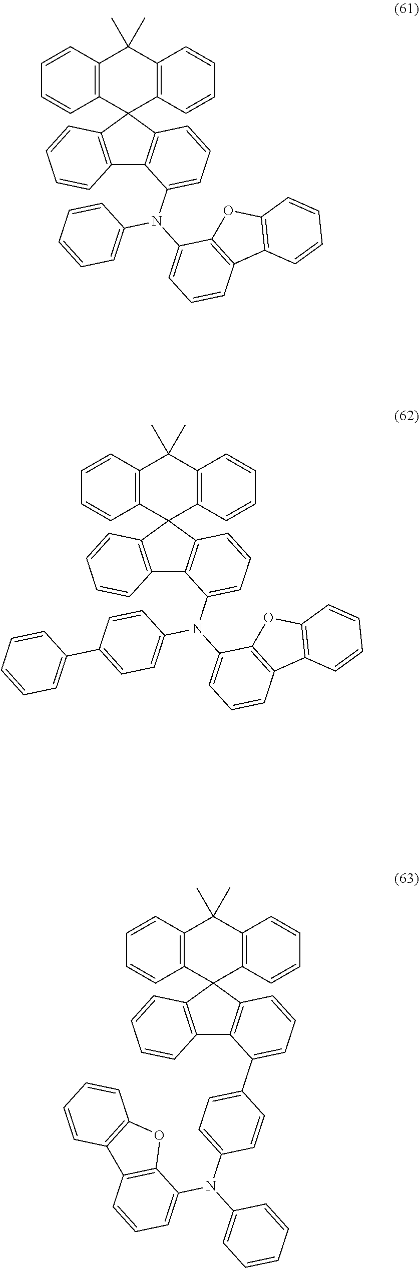 [SMILES: CC1(C)c2ccccc2C2(c3ccccc3-c3c(N(c4ccccc4)c4cccc5c4oc4ccccc45)cccc32)c2ccccc21 ; CC1(C)c2ccccc2C2(c3ccccc3-c3c(N(c4ccc(-c5ccccc5)cc4)c4cccc5c4oc4ccccc45)cccc32)c2ccccc21 ; CC1(C)c2ccccc2C2(c3ccccc3-c3c(-c4ccc(N(c5ccccc5)c5cccc6c5oc5ccccc56)cc4)cccc32)c2ccccc21]

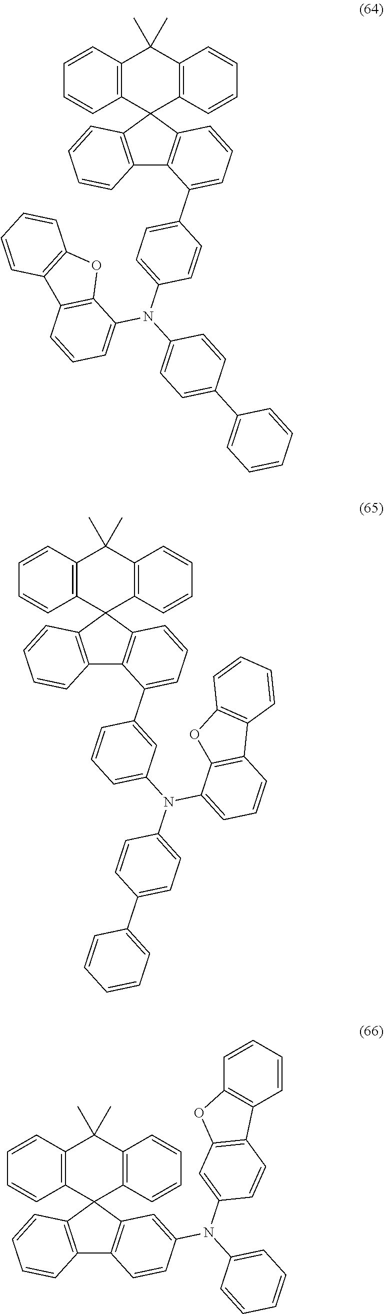 [SMILES: CC1(C)c2ccccc2C2(c3ccccc3-c3c(-c4ccc(N(c5ccc(-c6ccccc6)cc5)c5cccc6c5oc5ccccc56)cc4)cccc32)c2ccccc21 ; CC1(C)c2ccccc2C2(c3ccccc3-c3c(-c4cccc(N(c5ccc(-c6ccccc6)cc5)c5cccc6c5oc5ccccc56)c4)cccc32)c2ccccc21 ; CC1(C)c2ccccc2C2(c3ccccc3-c3ccc(N(c4ccccc4)c4ccc5c(c4)oc4ccccc45)cc32)c2ccccc21]

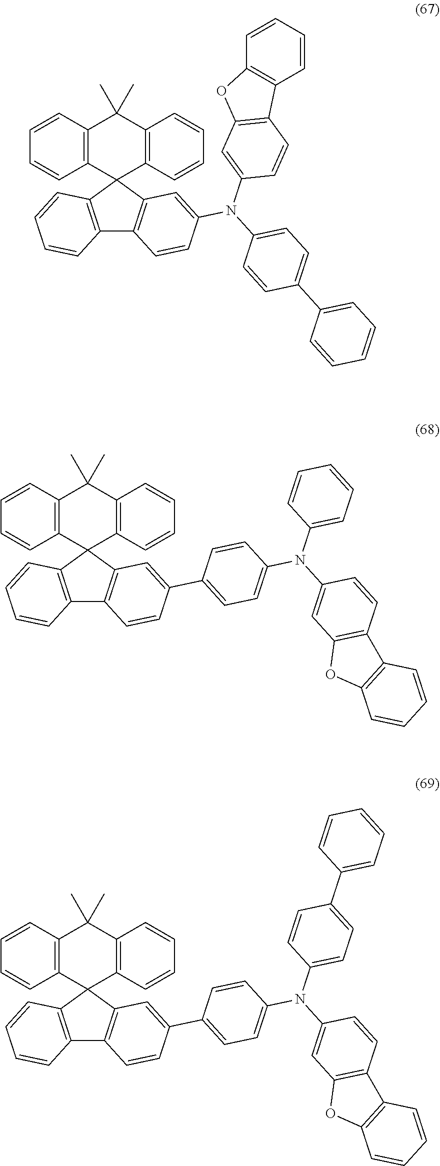 [SMILES: CC1(C)c2ccccc2C2(c3ccccc3-c3ccc(N(c4ccc(-c5ccccc5)cc4)c4ccc5c(c4)oc4ccccc45)cc32)c2ccccc21 ; CC1(C)c2ccccc2C2(c3ccccc3-c3ccc(-c4ccc(N(c5ccccc5)c5ccc6c(c5)oc5ccccc56)cc4)cc32)c2ccccc21 ; CC1(C)c2ccccc2C2(c3ccccc3-c3ccc(-c4ccc(N(c5ccc(-c6ccccc6)cc5)c5ccc6c(c5)oc5ccccc56)cc4)cc32)c2ccccc21]

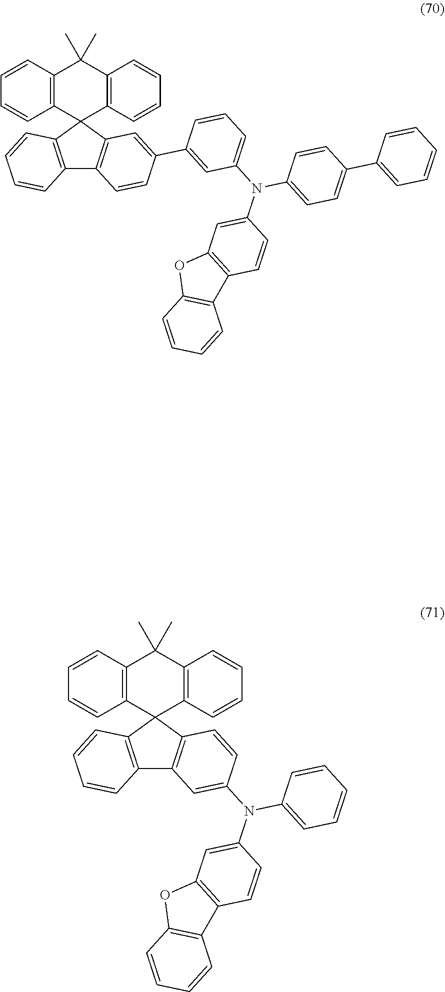 [SMILES: CC1(C)c2ccccc2C2(c3ccccc3-c3ccc(-c4cccc(N(c5ccc(-c6ccccc6)cc5)c5ccc6c(c5)oc5ccccc56)c4)cc32)c2ccccc21 ; CC1(C)c2ccccc2C2(c3ccccc3-c3cc(N(c4ccccc4)c4ccc5c(c4)oc4ccccc45)ccc32)c2ccccc21]

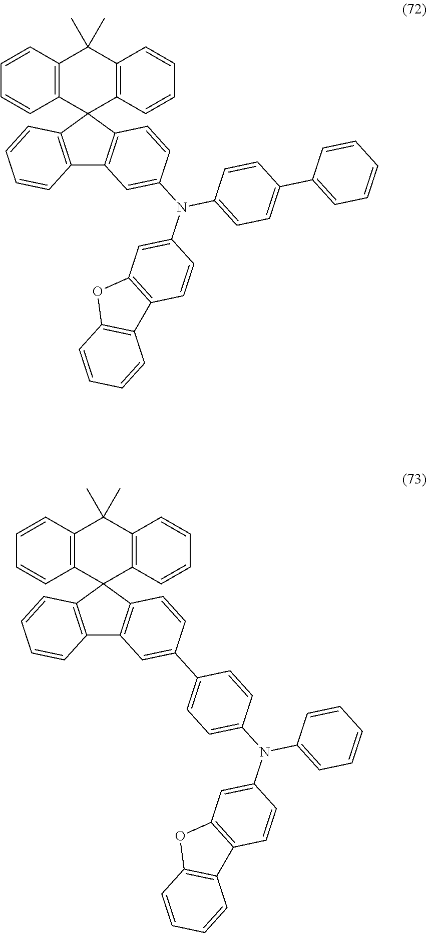 [SMILES: CC1(C)c2ccccc2C2(c3ccccc3-c3cc(N(c4ccc(-c5ccccc5)cc4)c4ccc5c(c4)oc4ccccc45)ccc32)c2ccccc21 ; CC1(C)c2ccccc2C2(c3ccccc3-c3cc(-c4ccc(N(c5ccccc5)c5ccc6c(c5)oc5ccccc56)cc4)ccc32)c2ccccc21]

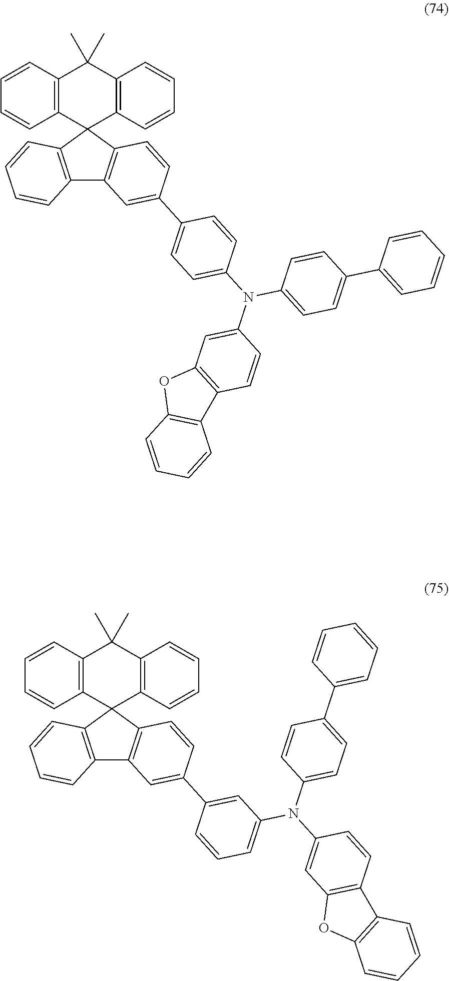 [SMILES: CC1(C)c2ccccc2C2(c3ccccc3-c3cc(-c4ccc(N(c5ccc(-c6ccccc6)cc5)c5ccc6c(c5)oc5ccccc56)cc4)ccc32)c2ccccc21 ; CC1(C)c2ccccc2C2(c3ccccc3-c3cc(-c4cccc(N(c5ccc(-c6ccccc6)cc5)c5ccc6c(c5)oc5ccccc56)c4)ccc32)c2ccccc21]

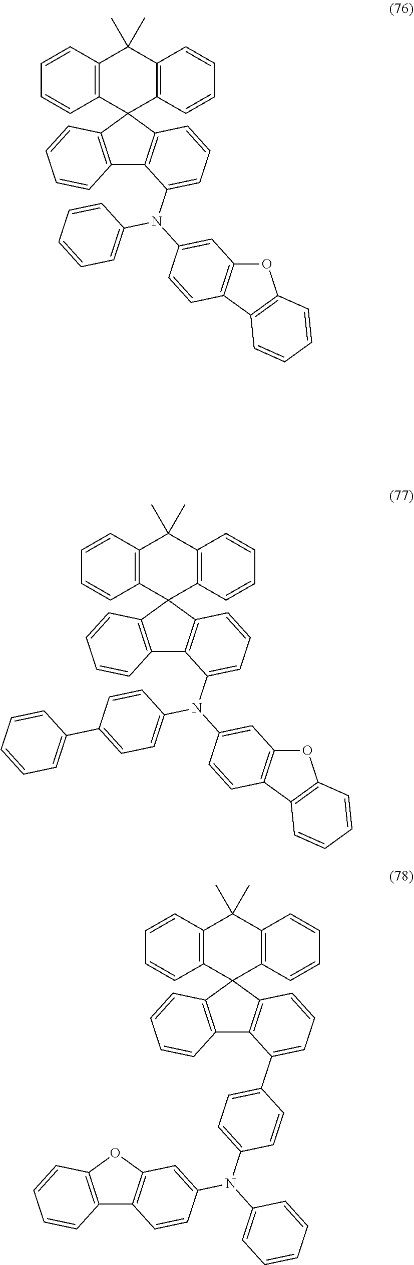 [SMILES: CC1(C)c2ccccc2C2(c3ccccc3-c3c(N(c4ccccc4)c4ccc5c(c4)oc4ccccc45)cccc32)c2ccccc21 ; CC1(C)c2ccccc2C2(c3ccccc3-c3c(N(c4ccc(-c5ccccc5)cc4)c4ccc5c(c4)oc4ccccc45)cccc32)c2ccccc21 ; CC1(C)c2ccccc2C2(c3ccccc3-c3c(-c4ccc(N(c5ccccc5)c5ccc6c(c5)oc5ccccc56)cc4)cccc32)c2ccccc21]

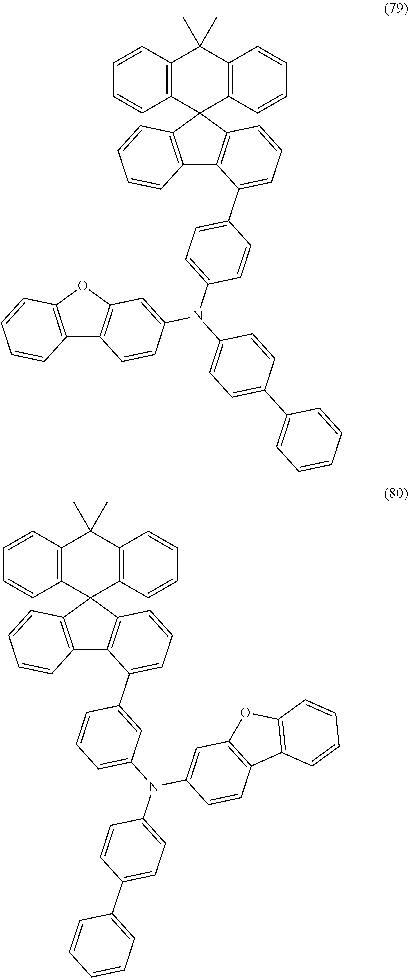 [SMILES: CC1(C)c2ccccc2C2(c3ccccc3-c3c(-c4ccc(N(c5ccc(-c6ccccc6)cc5)c5ccc6c(c5)oc5ccccc56)cc4)cccc32)c2ccccc21 ; CC1(C)c2ccccc2C2(c3ccccc3-c3c(-c4cccc(N(c5ccc(-c6ccccc6)cc5)c5ccc6c(c5)oc5ccccc56)c4)cccc32)c2ccccc21]

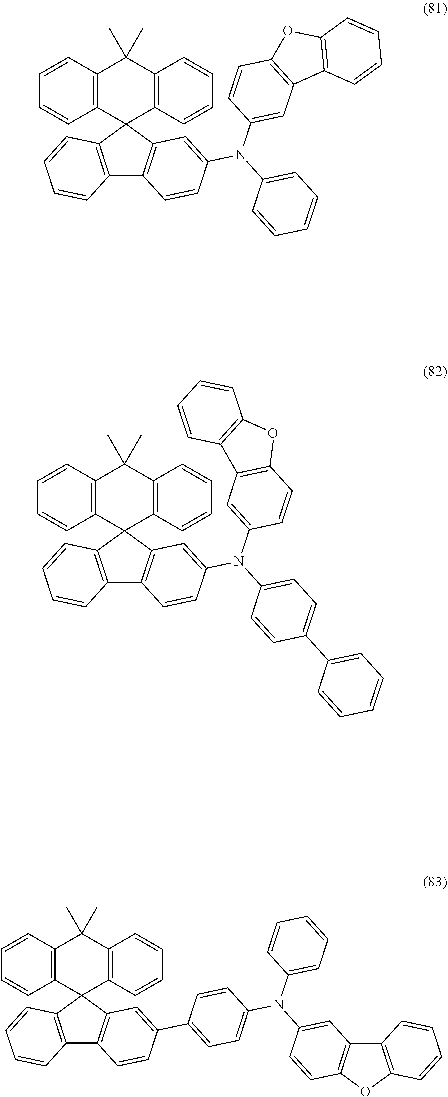 [SMILES: CC1(C)c2ccccc2C2(c3ccccc3-c3ccc(N(c4ccccc4)c4ccc5oc6ccccc6c5c4)cc32)c2ccccc21 ; CC1(C)c2ccccc2C2(c3ccccc3-c3ccc(N(c4ccc(-c5ccccc5)cc4)c4ccc5oc6ccccc6c5c4)cc32)c2ccccc21 ; CC1(C)c2ccccc2C2(c3ccccc3-c3ccc(-c4ccc(N(c5ccccc5)c5ccc6oc7ccccc7c6c5)cc4)cc32)c2ccccc21]

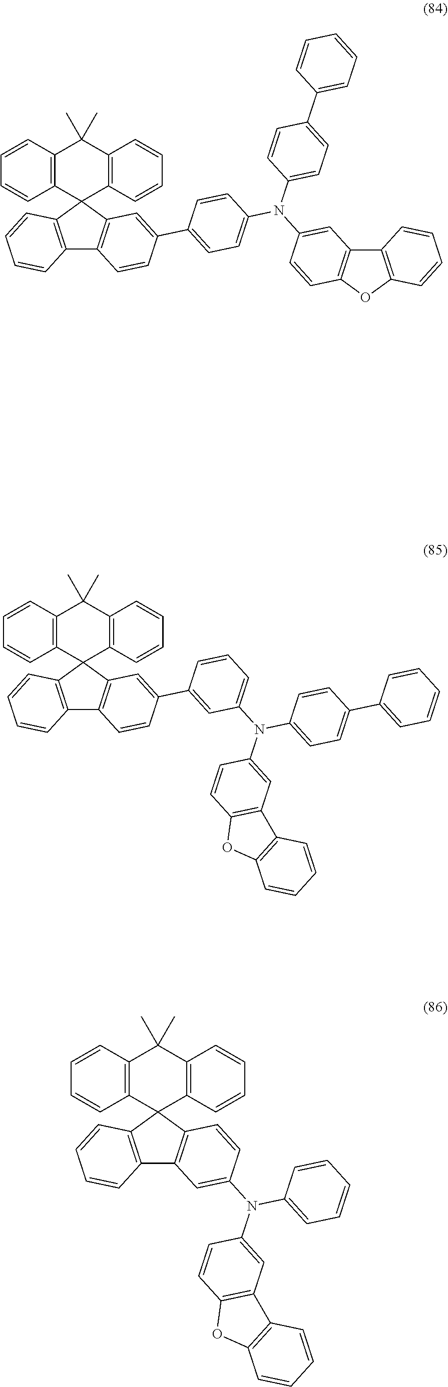 [SMILES: CC1(C)c2ccccc2C2(c3ccccc3-c3ccc(-c4ccc(N(c5ccc(-c6ccccc6)cc5)c5ccc6oc7ccccc7c6c5)cc4)cc32)c2ccccc21 ; CC1(C)c2ccccc2C2(c3ccccc3-c3ccc(-c4cccc(N(c5ccc(-c6ccccc6)cc5)c5ccc6oc7ccccc7c6c5)c4)cc32)c2ccccc21 ; CC1(C)c2ccccc2C2(c3ccccc3-c3cc(N(c4ccccc4)c4ccc5oc6ccccc6c5c4)ccc32)c2ccccc21]

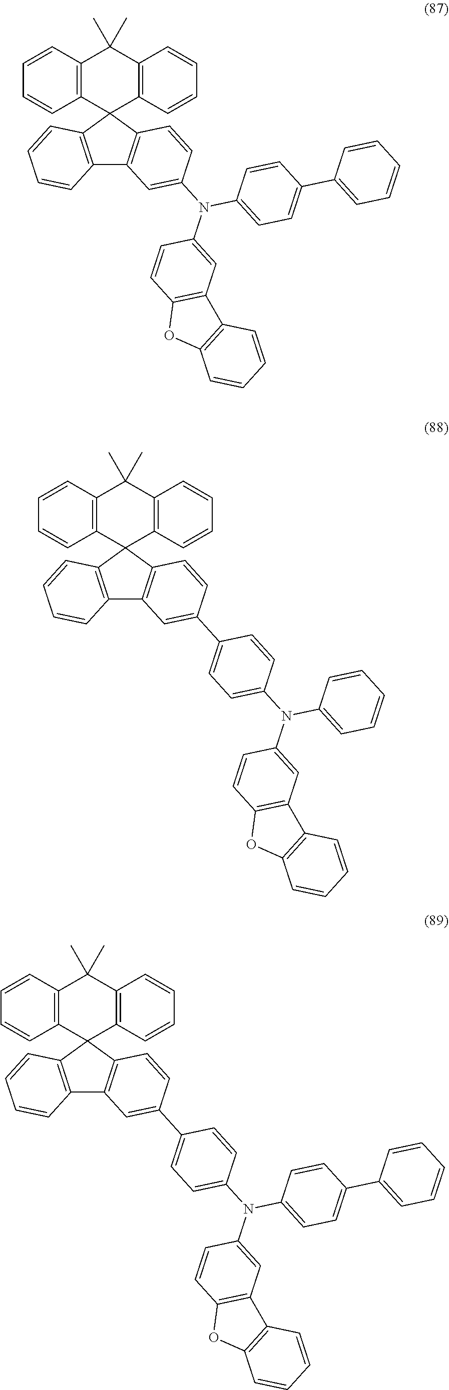 [SMILES: CC1(C)c2ccccc2C2(c3ccccc3-c3cc(N(c4ccc(-c5ccccc5)cc4)c4ccc5oc6ccccc6c5c4)ccc32)c2ccccc21 ; CC1(C)c2ccccc2C2(c3ccccc3-c3cc(-c4ccc(N(c5ccccc5)c5ccc6oc7ccccc7c6c5)cc4)ccc32)c2ccccc21 ; CC1(C)c2ccccc2C2(c3ccccc3-c3cc(-c4ccc(N(c5ccc(-c6ccccc6)cc5)c5ccc6oc7ccccc7c6c5)cc4)ccc32)c2ccccc21]

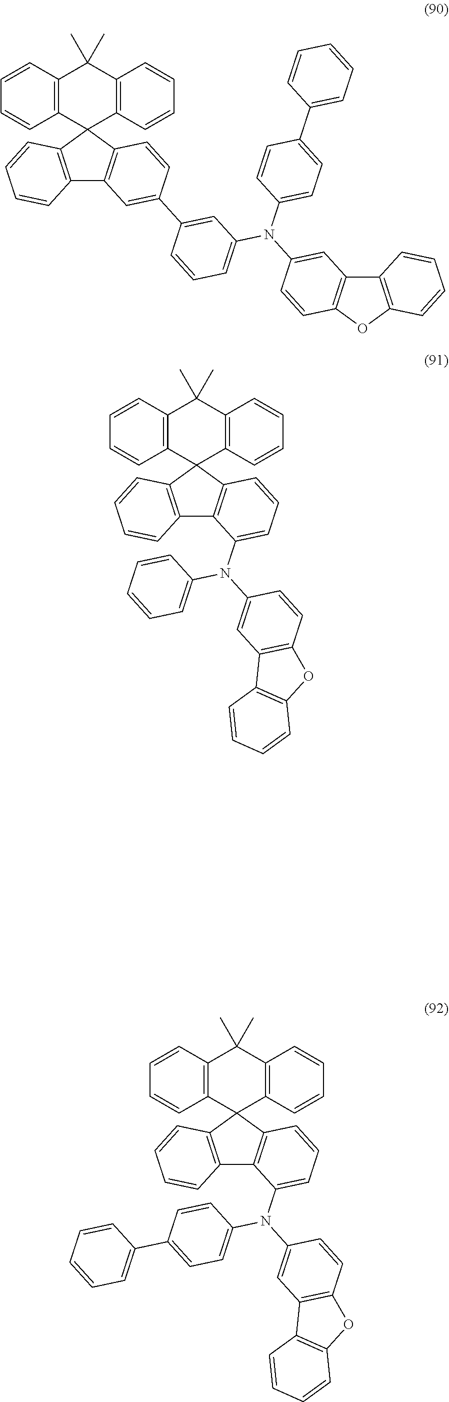 [SMILES: CC1(C)c2ccccc2C2(c3ccccc3-c3cc(-c4cccc(N(c5ccc(-c6ccccc6)cc5)c5ccc6oc7ccccc7c6c5)c4)ccc32)c2ccccc21 ; CC1(C)c2ccccc2C2(c3ccccc3-c3c(N(c4ccccc4)c4ccc5oc6ccccc6c5c4)cccc32)c2ccccc21 ; CC1(C)c2ccccc2C2(c3ccccc3-c3c(N(c4ccc(-c5ccccc5)cc4)c4ccc5oc6ccccc6c5c4)cccc32)c2ccccc21]

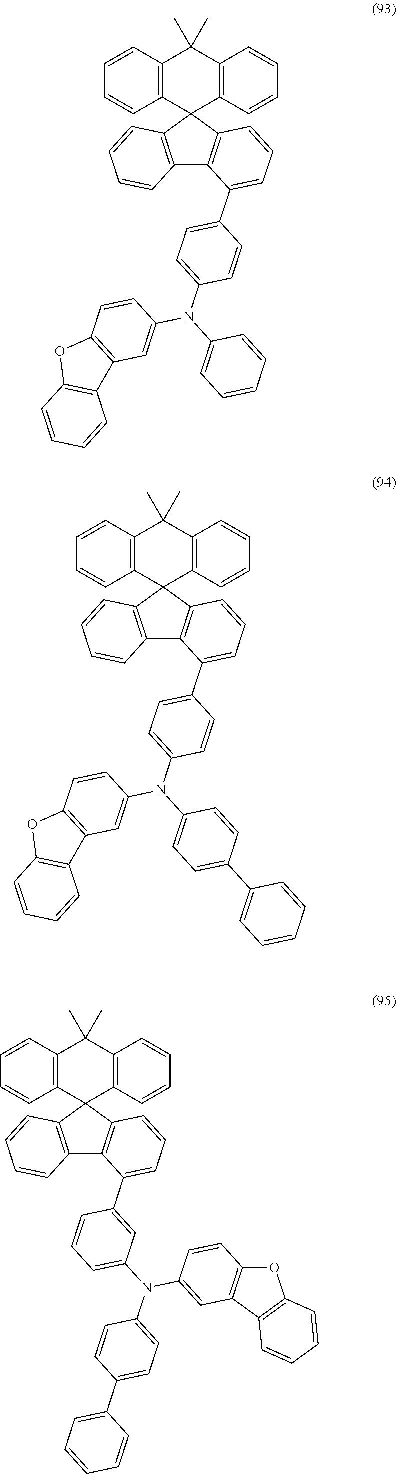 [SMILES: CC1(C)c2ccccc2C2(c3ccccc3-c3c(-c4ccc(N(c5ccccc5)c5ccc6oc7ccccc7c6c5)cc4)cccc32)c2ccccc21 ; CC1(C)c2ccccc2C2(c3ccccc3-c3c(-c4ccc(N(c5ccc(-c6ccccc6)cc5)c5ccc6oc7ccccc7c6c5)cc4)cccc32)c2ccccc21 ; CC1(C)c2ccccc2C2(c3ccccc3-c3c(-c4cccc(N(c5ccc(-c6ccccc6)cc5)c5ccc6oc7ccccc7c6c5)c4)cccc32)c2ccccc21]

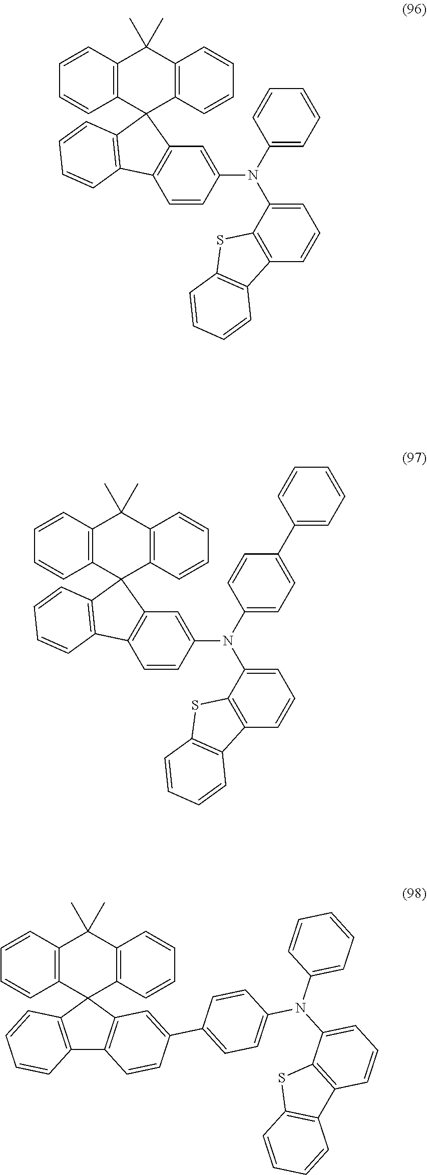 [SMILES: CC1(C)c2ccccc2C2(c3ccccc3-c3ccc(N(c4ccccc4)c4cccc5c4sc4ccccc45)cc32)c2ccccc21 ; CC1(C)c2ccccc2C2(c3ccccc3-c3ccc(N(c4ccc(-c5ccccc5)cc4)c4cccc5c4sc4ccccc45)cc32)c2ccccc21 ; CC1(C)c2ccccc2C2(c3ccccc3-c3ccc(-c4ccc(N(c5ccccc5)c5cccc6c5sc5ccccc56)cc4)cc32)c2ccccc21]

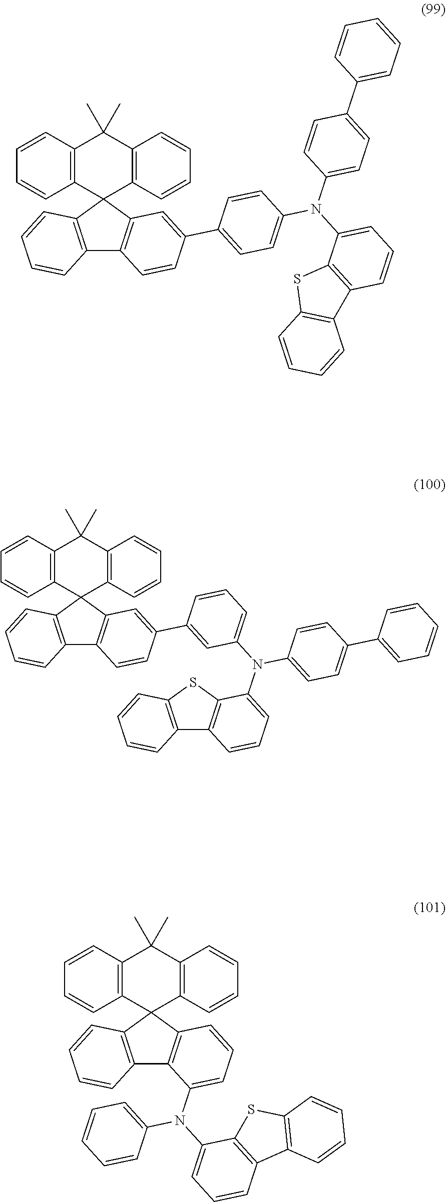 [SMILES: CC1(C)c2ccccc2C2(c3ccccc3-c3ccc(-c4ccc(N(c5ccc(-c6ccccc6)cc5)c5cccc6c5sc5ccccc56)cc4)cc32)c2ccccc21 ; CC1(C)c2ccccc2C2(c3ccccc3-c3ccc(-c4cccc(N(c5ccc(-c6ccccc6)cc5)c5cccc6c5sc5ccccc56)c4)cc32)c2ccccc21 ; CC1(C)c2ccccc2C2(c3ccccc3-c3c(N(c4ccccc4)c4cccc5c4sc4ccccc45)cccc32)c2ccccc21]

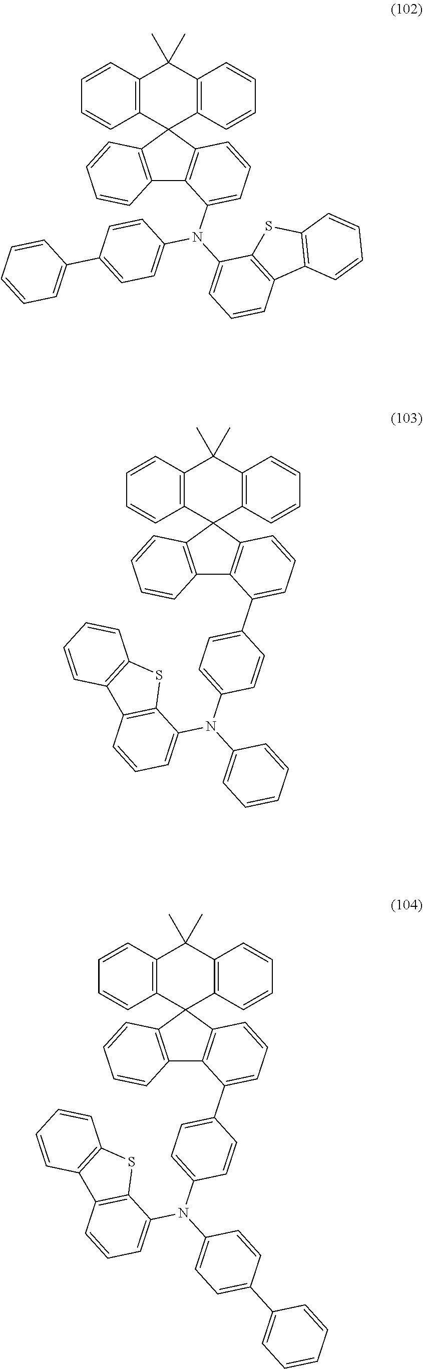 [SMILES: CC1(C)c2ccccc2C2(c3ccccc3-c3c(N(c4ccc(-c5ccccc5)cc4)c4cccc5c4sc4ccccc45)cccc32)c2ccccc21 ; CC1(C)c2ccccc2C2(c3ccccc3-c3c(-c4ccc(N(c5ccccc5)c5cccc6c5sc5ccccc56)cc4)cccc32)c2ccccc21 ; CC1(C)c2ccccc2C2(c3ccccc3-c3c(-c4ccc(N(c5ccc(-c6ccccc6)cc5)c5cccc6c5sc5ccccc56)cc4)cccc32)c2ccccc21]

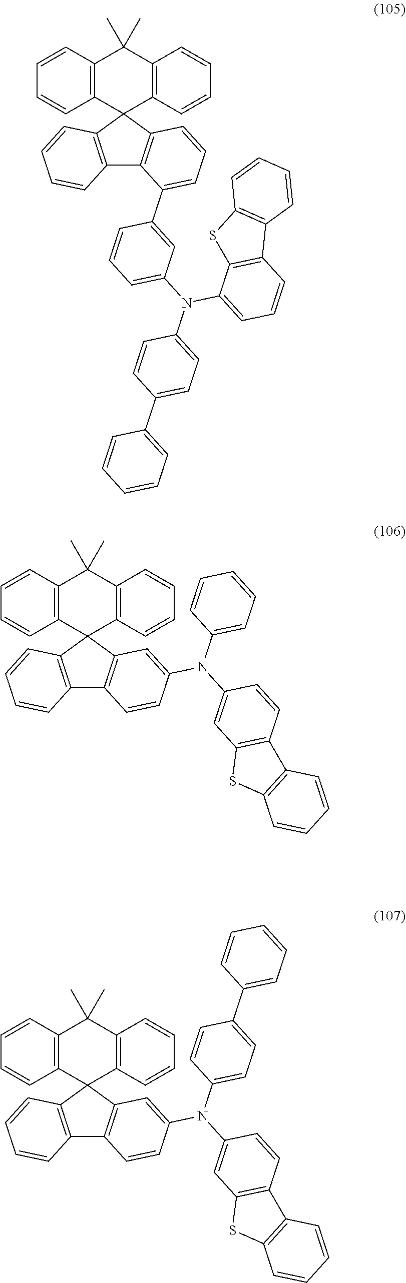 [SMILES: CC1(C)c2ccccc2C2(c3ccccc3-c3c(-c4cccc(N(c5ccc(-c6ccccc6)cc5)c5cccc6c5sc5ccccc56)c4)cccc32)c2ccccc21 ; CC1(C)c2ccccc2C2(c3ccccc3-c3ccc(N(c4ccccc4)c4ccc5c(c4)sc4ccccc45)cc32)c2ccccc21 ; CC1(C)c2ccccc2C2(c3ccccc3-c3ccc(N(c4ccc(-c5ccccc5)cc4)c4ccc5c(c4)sc4ccccc45)cc32)c2ccccc21]

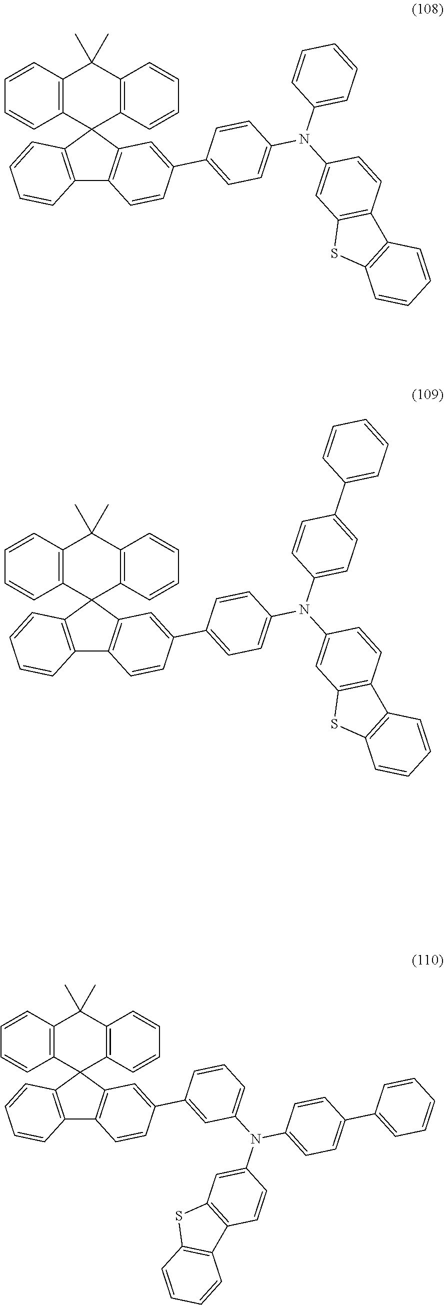 [SMILES: CC1(C)c2ccccc2C2(c3ccccc3-c3ccc(-c4ccc(N(c5ccccc5)c5ccc6c(c5)sc5ccccc56)cc4)cc32)c2ccccc21 ; CC1(C)c2ccccc2C2(c3ccccc3-c3ccc(-c4ccc(N(c5ccc(-c6ccccc6)cc5)c5ccc6c(c5)sc5ccccc56)cc4)cc32)c2ccccc21 ; CC1(C)c2ccccc2C2(c3ccccc3-c3ccc(-c4cccc(N(c5ccc(-c6ccccc6)cc5)c5ccc6c(c5)sc5ccccc56)c4)cc32)c2ccccc21]

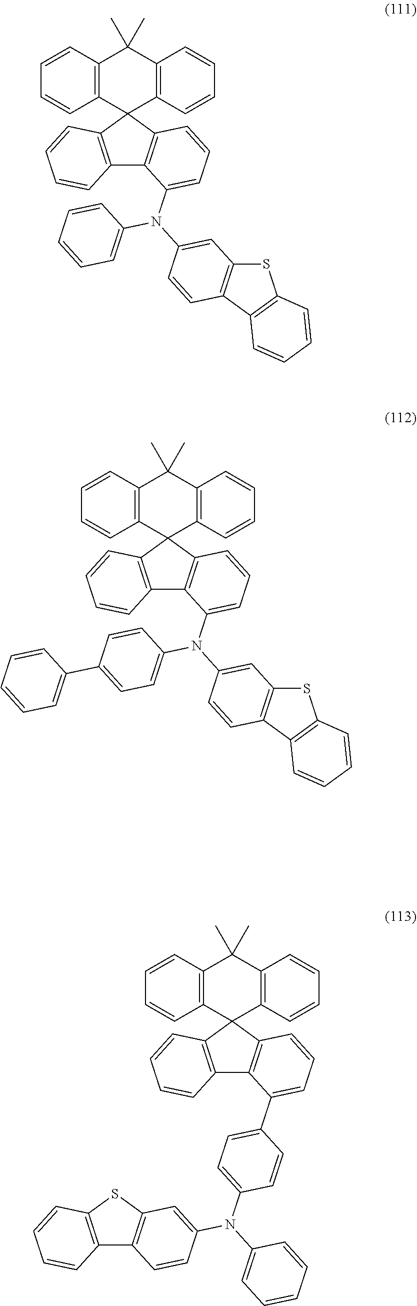 [SMILES: CC1(C)c2ccccc2C2(c3ccccc3-c3c(N(c4ccccc4)c4ccc5c(c4)sc4ccccc45)cccc32)c2ccccc21 ; CC1(C)c2ccccc2C2(c3ccccc3-c3c(N(c4ccc(-c5ccccc5)cc4)c4ccc5c(c4)sc4ccccc45)cccc32)c2ccccc21 ; CC1(C)c2ccccc2C2(c3ccccc3-c3c(-c4ccc(N(c5ccccc5)c5ccc6c(c5)sc5ccccc56)cc4)cccc32)c2ccccc21]

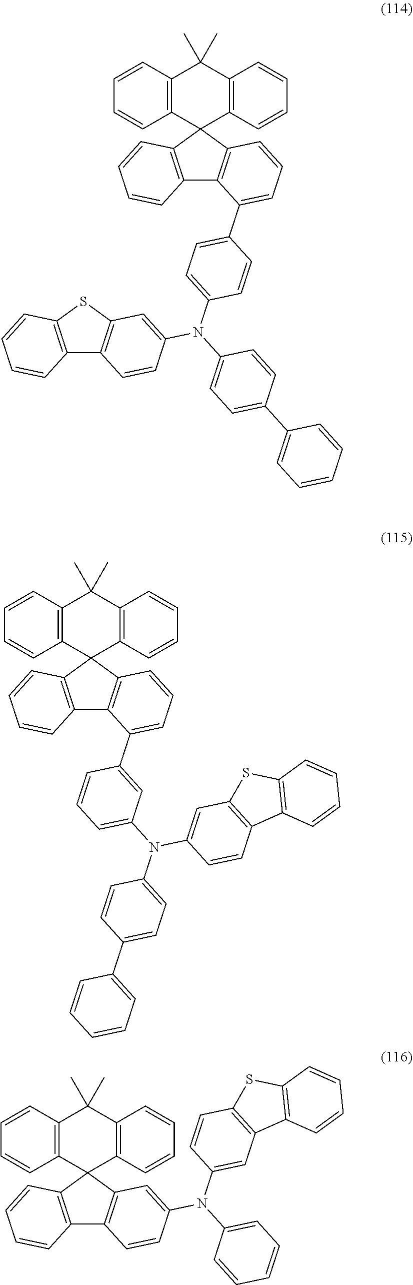

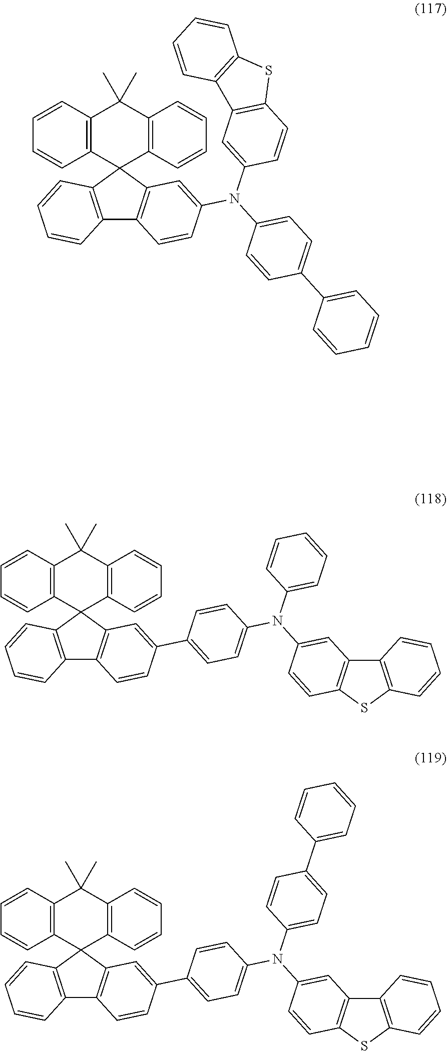 [SMILES: CC1(C)c2ccccc2C2(c3ccccc3-c3ccc(N(c4ccc(-c5ccccc5)cc4)c4ccc5sc6ccccc6c5c4)cc32)c2ccccc21 ; CC1(C)c2ccccc2C2(c3ccccc3-c3ccc(-c4ccc(N(c5ccccc5)c5ccc6sc7ccccc7c6c5)cc4)cc32)c2ccccc21 ; CC1(C)c2ccccc2C2(c3ccccc3-c3ccc(-c4ccc(N(c5ccc(-c6ccccc6)cc5)c5ccc6sc7ccccc7c6c5)cc4)cc32)c2ccccc21]

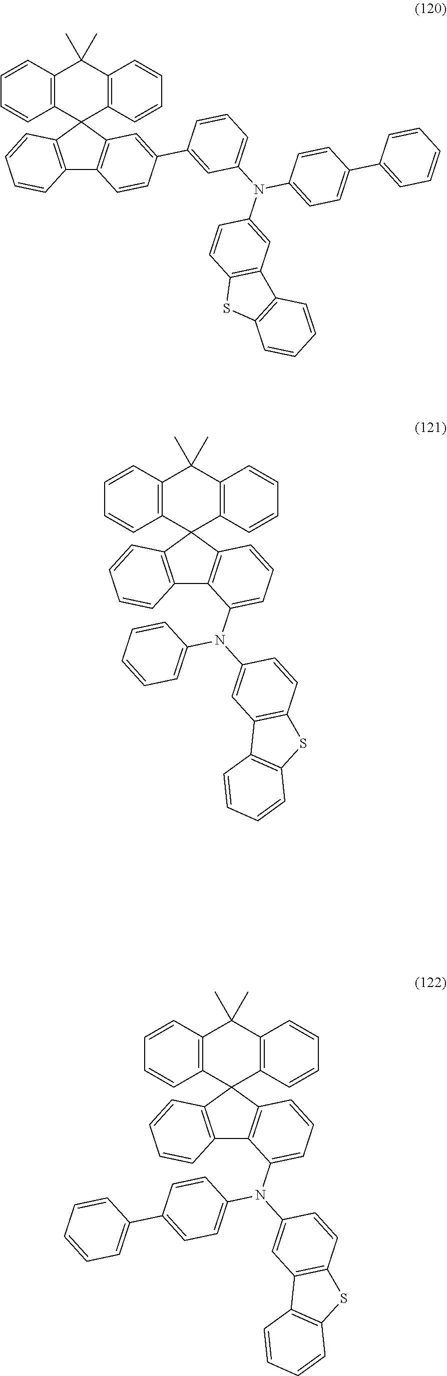 [SMILES: CC1(C)c2ccccc2C2(c3ccccc3-c3ccc(-c4cccc(N(c5ccc(-c6ccccc6)cc5)c5ccc6sc7ccccc7c6c5)c4)cc32)c2ccccc21 ; CC1(C)c2ccccc2C2(c3ccccc3-c3c(N(c4ccccc4)c4ccc5sc6ccccc6c5c4)cccc32)c2ccccc21 ; CC1(C)c2ccccc2C2(c3ccccc3-c3c(N(c4ccc(-c5ccccc5)cc4)c4ccc5sc6ccccc6c5c4)cccc32)c2ccccc21]

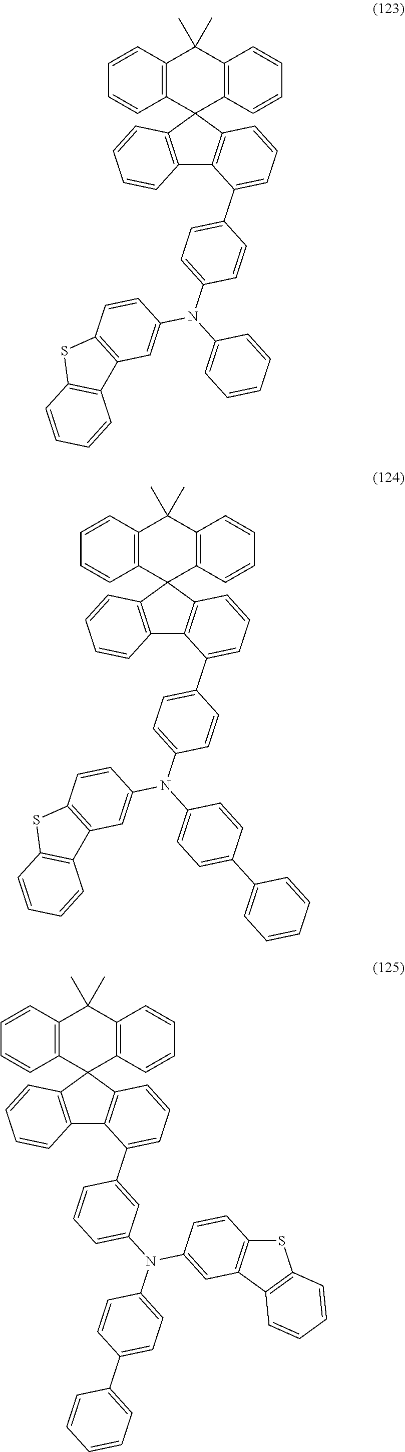 [SMILES: CC1(C)c2ccccc2C2(c3ccccc3-c3c(-c4ccc(N(c5ccccc5)c5ccc6sc7ccccc7c6c5)cc4)cccc32)c2ccccc21 ; CC1(C)c2ccccc2C2(c3ccccc3-c3c(-c4ccc(N(c5ccc(-c6ccccc6)cc5)c5ccc6sc7ccccc7c6c5)cc4)cccc32)c2ccccc21 ; CC1(C)c2ccccc2C2(c3ccccc3-c3c(-c4cccc(N(c5ccc(-c6ccccc6)cc5)c5ccc6sc7ccccc7c6c5)c4)cccc32)c2ccccc21]

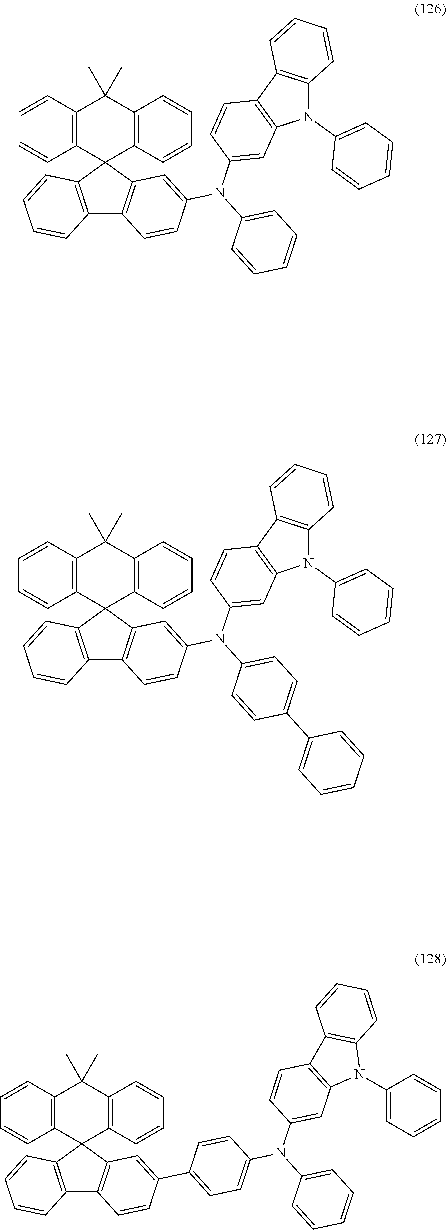 [SMILES: C=CC1=C(C=C)C2(c3ccccc3-c3ccc(N(c4ccccc4)c4ccc5c6ccccc6n(-c6ccccc6)c5c4)cc32)c2ccccc2C1(C)C ; CC1(C)c2ccccc2C2(c3ccccc3-c3ccc(N(c4ccc(-c5ccccc5)cc4)c4ccc5c6ccccc6n(-c6ccccc6)c5c4)cc32)c2ccccc21 ; CC1(C)c2ccccc2C2(c3ccccc3-c3ccc(-c4ccc(N(c5ccccc5)c5ccc6c7ccccc7n(-c7ccccc7)c6c5)cc4)cc32)c2ccccc21]

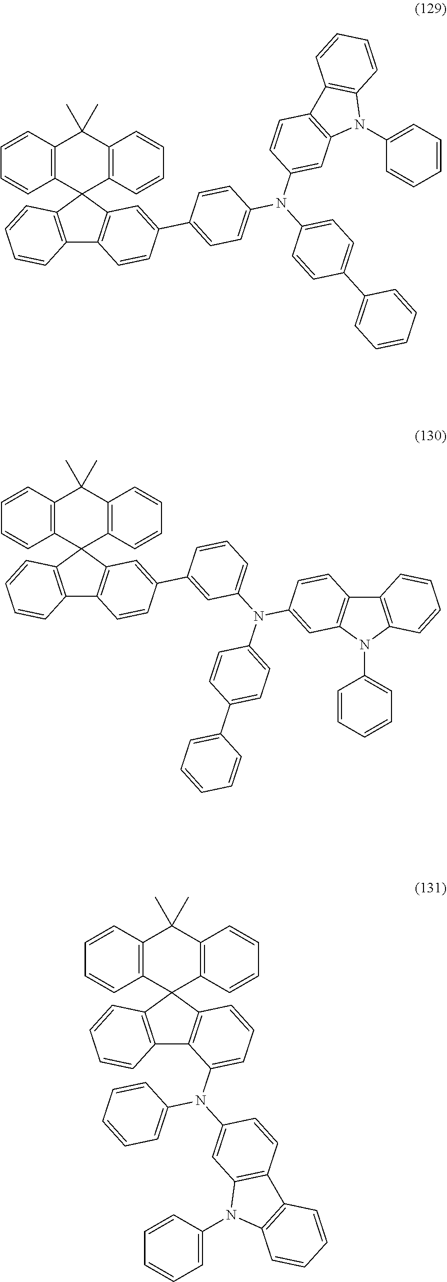 [SMILES: CC1(C)c2ccccc2C2(c3ccccc3-c3ccc(-c4ccc(N(c5ccc(-c6ccccc6)cc5)c5ccc6c7ccccc7n(-c7ccccc7)c6c5)cc4)cc32)c2ccccc21 ; CC1(C)c2ccccc2C2(c3ccccc3-c3ccc(-c4cccc(N(c5ccc(-c6ccccc6)cc5)c5ccc6c7ccccc7n(-c7ccccc7)c6c5)c4)cc32)c2ccccc21 ; CC1(C)c2ccccc2C2(c3ccccc3-c3c(N(c4ccccc4)c4ccc5c6ccccc6n(-c6ccccc6)c5c4)cccc32)c2ccccc21]

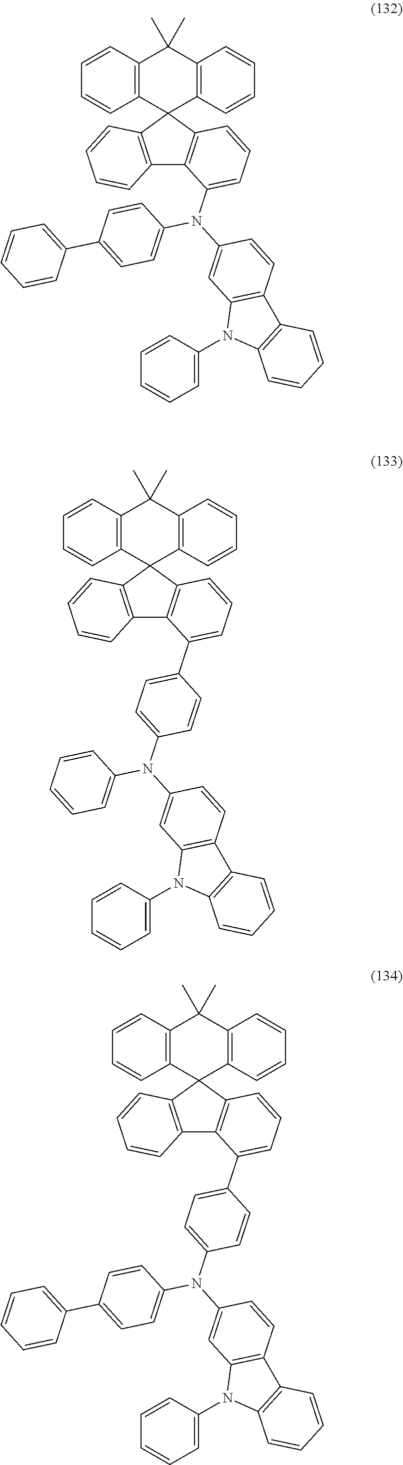 [SMILES: CC1(C)c2ccccc2C2(c3ccccc3-c3c(N(c4ccc(-c5ccccc5)cc4)c4ccc5c6ccccc6n(-c6ccccc6)c5c4)cccc32)c2ccccc21 ; CC1(C)c2ccccc2C2(c3ccccc3-c3c(-c4ccc(N(c5ccccc5)c5ccc6c7ccccc7n(-c7ccccc7)c6c5)cc4)cccc32)c2ccccc21 ; CC1(C)c2ccccc2C2(c3ccccc3-c3c(-c4ccc(N(c5ccc(-c6ccccc6)cc5)c5ccc6c7ccccc7n(-c7ccccc7)c6c5)cc4)cccc32)c2ccccc21]

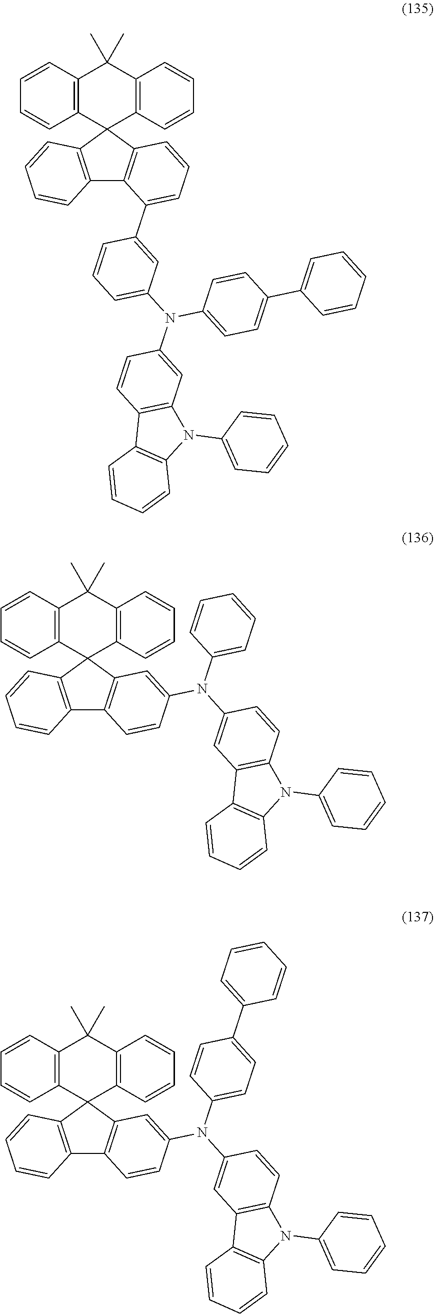 [SMILES: CC1(C)c2ccccc2C2(c3ccccc3-c3c(-c4cccc(N(c5ccc(-c6ccccc6)cc5)c5ccc6c7ccccc7n(-c7ccccc7)c6c5)c4)cccc32)c2ccccc21 ; CC1(C)c2ccccc2C2(c3ccccc3-c3ccc(N(c4ccccc4)c4ccc5c(c4)c4ccccc4n5-c4ccccc4)cc32)c2ccccc21 ; CC1(C)c2ccccc2C2(c3ccccc3-c3ccc(N(c4ccc(-c5ccccc5)cc4)c4ccc5c(c4)c4ccccc4n5-c4ccccc4)cc32)c2ccccc21]

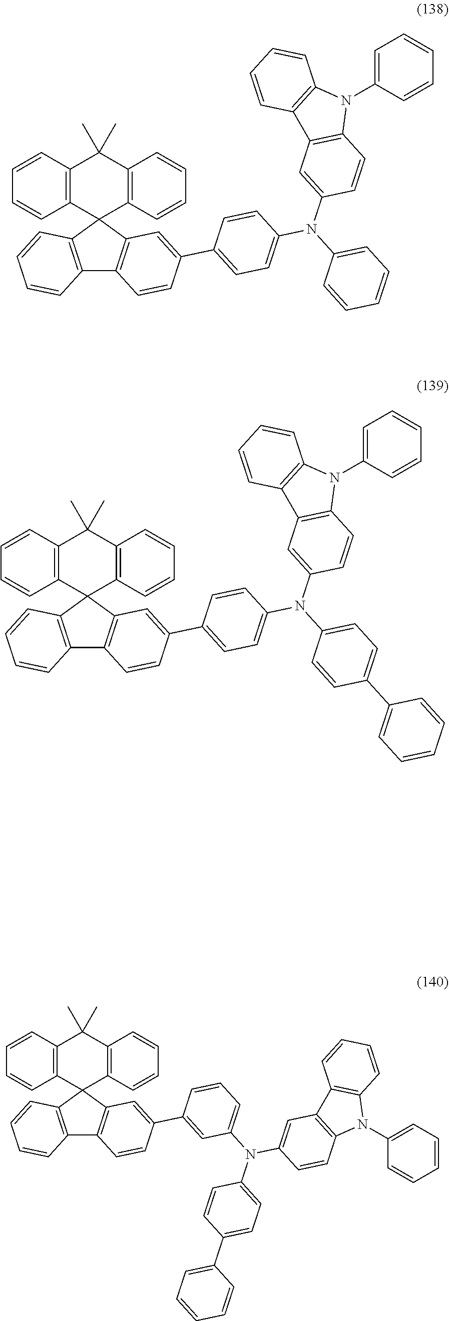 [SMILES: CC1(C)c2ccccc2C2(c3ccccc3-c3ccc(-c4ccc(N(c5ccccc5)c5ccc6c(c5)c5ccccc5n6-c5ccccc5)cc4)cc32)c2ccccc21 ; CC1(C)c2ccccc2C2(c3ccccc3-c3ccc(-c4ccc(N(c5ccc(-c6ccccc6)cc5)c5ccc6c(c5)c5ccccc5n6-c5ccccc5)cc4)cc32)c2ccccc21 ; CC1(C)c2ccccc2C2(c3ccccc3-c3ccc(-c4cccc(N(c5ccc(-c6ccccc6)cc5)c5ccc6c(c5)c5ccccc5n6-c5ccccc5)c4)cc32)c2ccccc21]

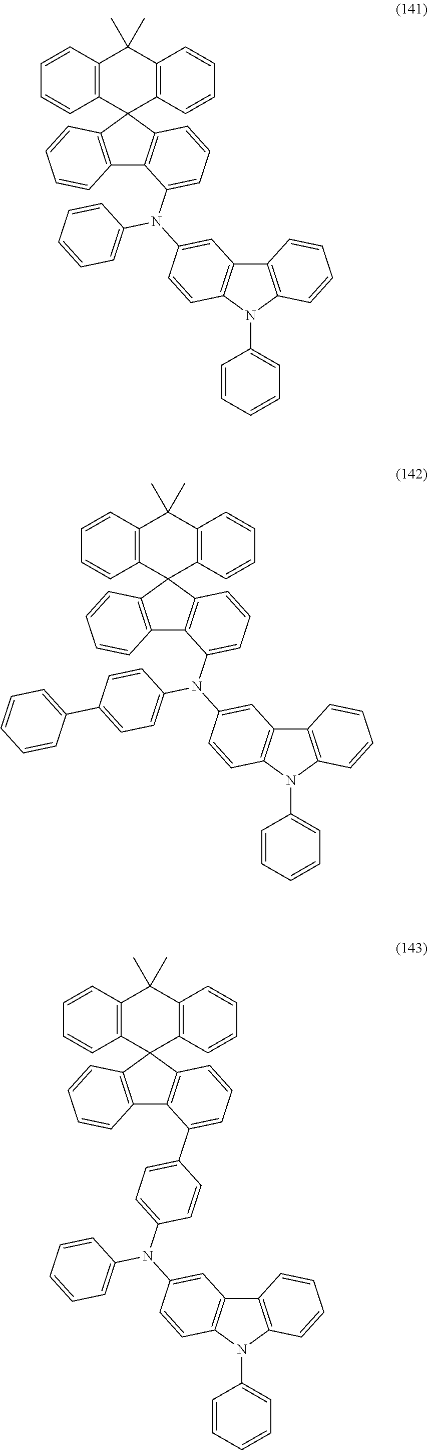 [SMILES: CC1(C)c2ccccc2C2(c3ccccc3-c3c(N(c4ccccc4)c4ccc5c(c4)c4ccccc4n5-c4ccccc4)cccc32)c2ccccc21 ; CC1(C)c2ccccc2C2(c3ccccc3-c3c(N(c4ccc(-c5ccccc5)cc4)c4ccc5c(c4)c4ccccc4n5-c4ccccc4)cccc32)c2ccccc21 ; CC1(C)c2ccccc2C2(c3ccccc3-c3c(-c4ccc(N(c5ccccc5)c5ccc6c(c5)c5ccccc5n6-c5ccccc5)cc4)cccc32)c2ccccc21]

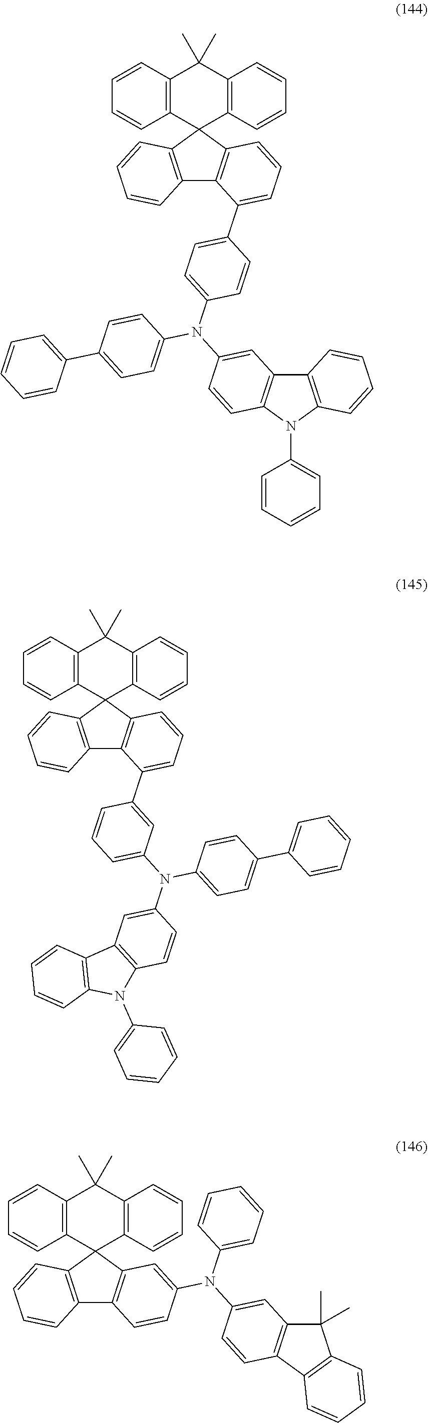 [SMILES: CC1(C)c2ccccc2C2(c3ccccc3-c3c(-c4ccc(N(c5ccc(-c6ccccc6)cc5)c5ccc6c(c5)c5ccccc5n6-c5ccccc5)cc4)cccc32)c2ccccc21 ; CC1(C)c2ccccc2C2(c3ccccc3-c3c(-c4cccc(N(c5ccc(-c6ccccc6)cc5)c5ccc6c(c5)c5ccccc5n6-c5ccccc5)c4)cccc32)c2ccccc21 ; CC1(C)c2ccccc2-c2ccc(N(c3ccccc3)c3ccc4c(c3)C3(c5ccccc5-4)c4ccccc4C(C)(C)c4ccccc43)cc21]

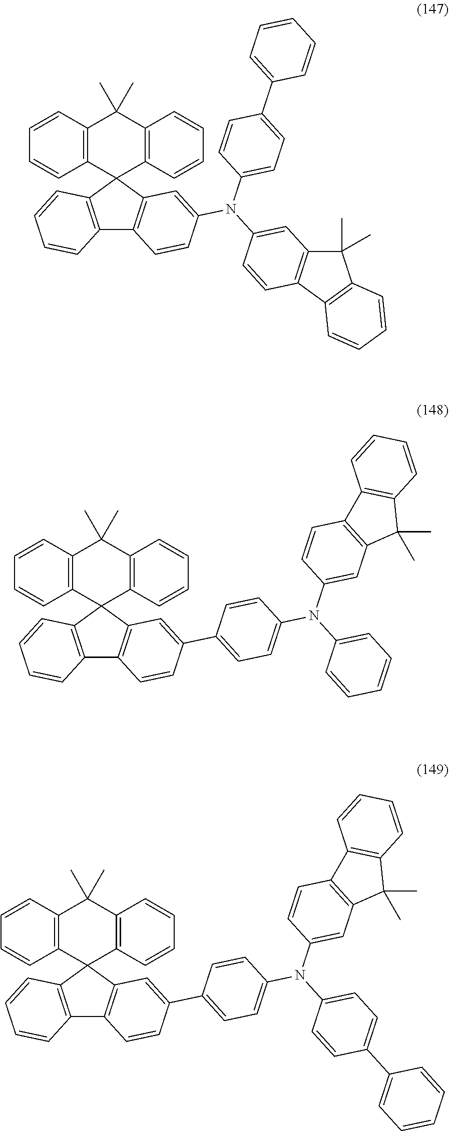 [SMILES: CC1(C)c2ccccc2-c2ccc(N(c3ccc(-c4ccccc4)cc3)c3ccc4c(c3)C3(c5ccccc5-4)c4ccccc4C(C)(C)c4ccccc43)cc21 ; CC1(C)c2ccccc2-c2ccc(N(c3ccccc3)c3ccc(-c4ccc5c(c4)C4(c6ccccc6-5)c5ccccc5C(C)(C)c5ccccc54)cc3)cc21 ; CC1(C)c2ccccc2-c2ccc(N(c3ccc(-c4ccccc4)cc3)c3ccc(-c4ccc5c(c4)C4(c6ccccc6-5)c5ccccc5C(C)(C)c5ccccc54)cc3)cc21]

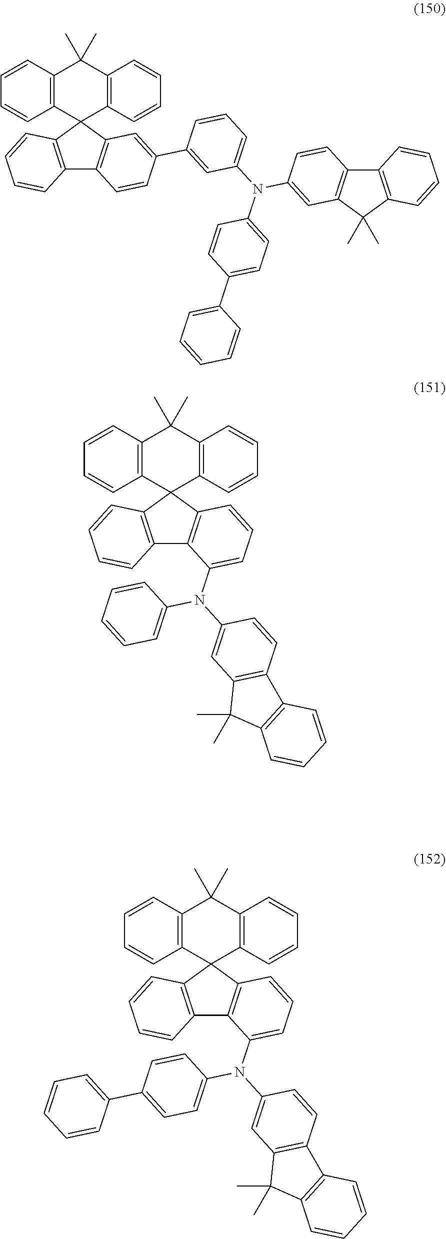

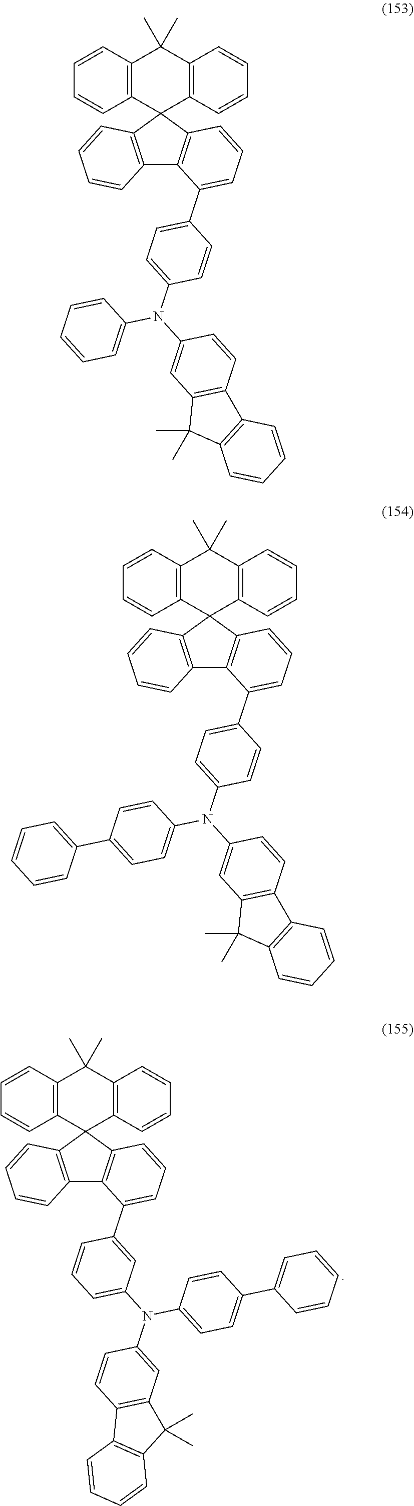

The compound of Chemical Formula 1 of the present invention may be synthesized using general synthesis methods (refer to Chem. Rev., 60:313 (1960); J. Chem. SOC. 4482 (1955); Chem. Rev. 95: 2457 (1995) or the like). Detailed synthesis processes of the compounds of the present invention will be specifically described in synthesis examples to be described later.

2. Organic Electroluminescent Device

Meanwhile, another aspect of the present invention relates to an organic electroluminescent device (organic EL device) including the compound represented by Chemical Formula 1 according to the present invention.

Specifically, the present invention relates to an organic electroluminescent device including an anode, a cathode, and one or more organic material layers provided between the anode and the cathode, and at least one of the one or more organic material layers includes the compound represented by Chemical Formula 1. Herein, the compound may be used either alone or as a mixture of two or more.

The one or more organic material layers may be any one or more of a hole injection layer, a hole transport layer, a light emitting layer, a light emitting auxiliary layer, a lifetime improving layer, an electron transport layer, an electron transport auxiliary layer and an electron injection layer, and at least one organic material layer among these may include the compound represented by Chemical Formula 1.

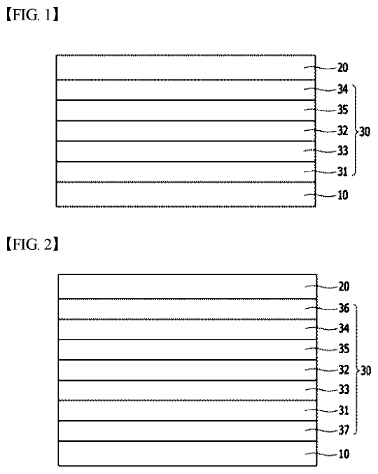

The structure of the organic electroluminescent device according to the present invention described above is not particularly limited, but, when referring to FIG. 1 as one example, includes an anode (10) and a cathode (20) facing each other, and an organic layer (30) located between the anode (10) and the cathode (20). Herein, the organic layer (30) may include a hole transport layer (31), a light emitting layer (32) and an electron transport layer (34). In addition, a hole transport auxiliary layer (33) may be included between the hole transport layer (31) and the light emitting layer (32), and an electron transport auxiliary layer (35) may be included between the electron transport layer (34) and the light emitting layer (32).

When referring to FIG. 2 as another example of the present invention, the organic layer (30) may further include a hole injection layer (37) between the hole transport layer (31) and the anode (10), and may further include an electron injection layer (36) between the electron transport layer (34) and the cathode (20).

The hole injection layer (37) laminated between the hole transport layer (31) and the anode (10) in the present invention is a layer having a function of, as well as improving interfacial properties between ITO used as the anode and an organic material used as the hole transport layer (31), smoothing the ITO surface by being coated on the top of the ITO of which surface is not smooth, and those commonly used in the art may be used without particular limit, and for example, amine compounds may be used. However, the hole injection layer is not limited thereto.

In addition, the electron injection layer (36) is a layer laminated on the top of the electron transport layer (34) and having a function of facilitating electron injection from the cathode and eventually improving power efficiency, and is not particularly limited as long as it is commonly used in the art. For example, materials such as LiF, Liq, NaCl, CsF, Li₂O or BaO may be used.

Although not shown in the drawings in the present invention, a light emitting auxiliary layer may be further included between the hole transport auxiliary layer (33) and the light emitting layer (32). The light emitting auxiliary layer may perform a role of adjusting a thickness of the organic layer (30) while performing a role of transporting holes to the light emitting layer (32). The light emitting auxiliary layer may include a hole transport material, and may be formed with the same material as the hole transport layer (31).

In addition, although not shown in the drawings in the present invention, a lifetime improving layer may be further included between the electron transport auxiliary layer (35) and the light emitting layer (32). Holes migrating to the light emitting layer (32) by getting on an ionization potential level in an organic light emitting device are not able to diffuse or migrate to the electron transport layer by being blocked by a high energy barrier of the lifetime improving layer, and consequently, the lifetime improving layer has a function of limiting the holes in the light emitting layer. Such a function of limiting the holes in the light emitting layer prevents the holes from diffusing to the electron transport layer migrating electrons by reduction, and therefore, suppresses a lifetime decrease phenomenon caused through an irreversible decomposition reaction by oxidation, and thereby contributes to improving a lifetime of the organic light emitting device.

Spiroacridine-based structures basically have very superior electrochemical stability, a high glass transition temperature and an excellent carrier transport ability, and particularly have a very superior hole transport ability, and thereby smoothly transport holes to a light emitting layer increasing luminous efficiency.

In the present invention, the compound represented by Chemical Formula 1 has structural characteristics of adding spirodimethylfluorene and arylamine, and has properties of low voltage driving and high refractive index, and as a result, physical properties of high efficiency and long lifetime are obtained.

Accordingly, the compound represented by Chemical Formula 1, a representative claimed structure of the present invention, has an excellent light emitting property, and may be used as a material of any one of a hole injection layer, a hole transport layer, a light emitting layer, an electron transport layer and an electron injection layer, which are organic material layers of an organic electroluminescent device. Preferably, the compound represented by Chemical Formula 1 may be used as a material of a hole transport layer and a hole transport auxiliary layer.

In addition, the organic electroluminescent device in the present invention has, as described above, an anode, one or more organic material layers and a cathode consecutively laminated, and in addition thereto, may further include an insulating layer or an adhesive layer at an interface between the electrode and the organic material layer.

Except that at least one or more of the organic material layers (for example, electron transport auxiliary layer) are formed to include the compound represented by Chemical Formula 1, the organic electroluminescent device of the present invention may be manufactured by forming other organic material layers and electrodes using materials and methods known in the art.

The organic material layer may be formed using a vacuum deposition method or a solution coating method. Examples of the solution coating method may include spin coating, dip coating, doctor blading, inkjet printing, thermal transfer method or the like, but are not limited thereto.

A substrate capable of being used in the present invention is not particularly limited, and silicon wafers, quartz, glass plates, metal plates, plastic films, sheets and the like may be used.

The anode material may be prepared using, for example, a conductor having high work function so as to have smooth hole injection, and examples thereof may include metals such as vanadium, chromium, copper, zinc or gold, or alloys thereof; metal oxides such as zinc oxide, indium oxide, indium tin oxide (ITO) or indium zinc oxide (IZO); combinations of metals and oxides such as ZnO:Al or SnO₂:Sb; conductive polymers such as polythiophene, poly(3-methylthiophene), poly[3,4-(ethylene-1,2-dioxy)thiophene] (PEDT), polypyrrole or polyaniline; carbon black, and the like, but are not limited thereto.

The cathode material may be prepared using, for example, a conductor having low work function so as to have smooth electron injection, and examples thereof may include metals such as magnesium, calcium, sodium, potassium, titanium, indium, yttrium, lithium, gadolinium, aluminum, silver, tin or lead, or alloys thereof; and multilayer-structured materials such as LiF/Al or LiO₂/Al, but are not limited thereto.

Hereinafter, the present invention will be described in detail with reference to examples as follows. However, the following examples are for illustrative purposes only, and the present invention is not limited to the following examples.

### EXAMPLE

### [Preparation Example 1] Synthesis of Core 1

### <Step 1> Synthesis of 9-(4′-chloro-[1,1′-biphenyl]-2-yl)-10,10-dimethyl-9,10-dihydroanthracen-9-ol

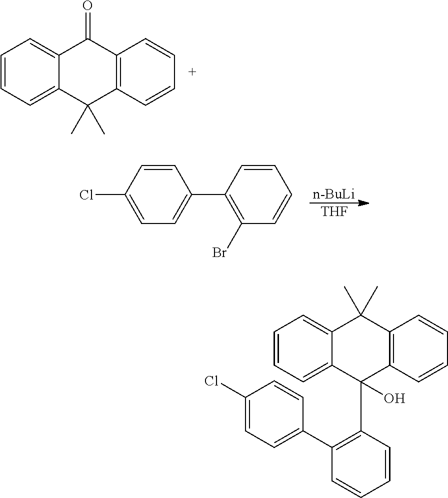

THF (500 mL) was added to 2-bromo-4′-chloro-1,1′-biphenyl (50 g, 0.19 mol). Then, the temperature of the reaction solution was lowered to −78° C., and a 1.6 M n-BuLi solution (128 mL, 0.21 mol) was slowly added dropwise to the reaction solution. After stirring the result for 1 hour at the same temperature, 10,10-dimethylanthracen-9(10H)-one (45.7 g, 0.21 mol) dissolved in THF (500 mL) was slowly added to the reaction solution, and the result was stirred for 1 hour at the same temperature, and then further stirred for 24 hours at room temperature. Then, purified water (500 mL) was introduced to the reaction solution to terminate the reaction, and then the result was extracted with E.A (2.0 L) and washed with distilled water. After that, the obtained organic layer was dried with anhydrous MgSO₄, vacuum distilled, and then purified using silica gel column chromatography to obtain a target compound (52.2 g, yield 68%).

¹H-NMR (in DMSO): δ 8.42 (d, 1H), 7.55 (t, 1H), 7.31 (m, 3H), 7.20 (m, 2H), 7.06 (m, 2H), 6.86 (d, 2H), 6.68 (d, 1H), 6.60 (d, 2H), 5.71 (s, 1H), 5.62 (d, 2H), 1.36 (s, 3H), 1.08 (s, 3H)

[LCMS]: 410

### <Step 2> Synthesis of 2′-chloro-10,10-dimethyl-10H-spiro[anthracene-9,9′-fluorene]

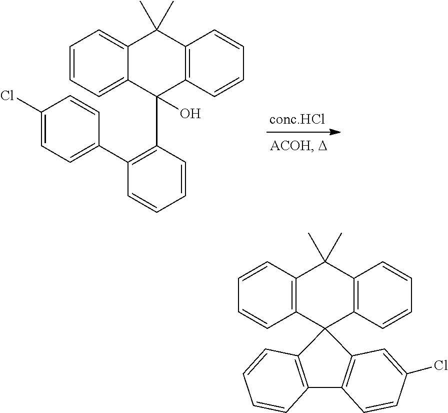 [SMILES: CC1(C)c2ccccc2C(O)(c2ccccc2-c2ccc(Cl)cc2)c2ccccc21 ; CC1(C)c2ccccc2C2(c3ccccc3-c3ccc(Cl)cc32)c2ccccc21]

Conc. HCl (70 mL) and AcOH (700 mL) were added to 9-(4′-chloro-[1,1′-biphenyl]-2-yl)-10,10-dimethyl-9,10-dihydroanthracen-9-ol (46.0 g, 0.11 mol). The reaction solution was heated under reflux for 2 hours at 100° C. The temperature was lowered to room temperature, and after introducing purified water (500 mL) to the reaction solution to terminate the reaction, the produced solids were vacuum filtered and dried using warm air to obtain a target compound (43.1 g, yield 98%).

¹H-NMR (in CDCl₃): δ 7.78 (d, 1H), 7.73 (d, 1H), 7.60 (dd, 2H), 7.30 (m, 2H), 7.20 (dt, 2H), 7.14 (dt, 1H), 6.86 (m, 4H), 6.28 (dd, 2H), 1.92 (s, 3H), 1.90 (s, 3H)

[LCMS]: 392

### [Preparation Example 2] Synthesis of Core 2

### <Step 1> Synthesis of 9-(3′-chloro-[1,1′-biphenyl]-2-yl)-10,10-dimethyl-9,10-dihydroanthracen-9-ol

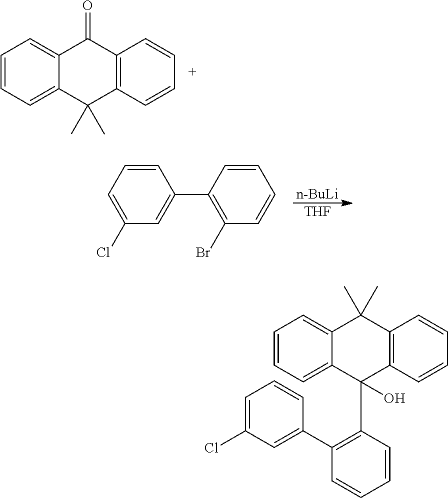

A target compound (49.9 g, 65%) corresponding to a structural isomer of Core 1 was obtained in the same manner as in Step 1 of [Preparation Example 1].

[LCMS]: 410

### <Step 2> Synthesis of 3′-chloro-10,10-dimethyl-10H-spiro[anthracene-9,9′-fluorene]

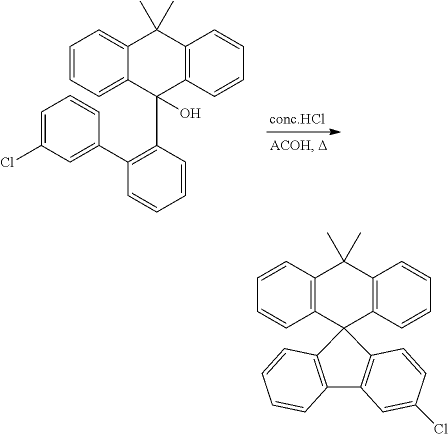 [SMILES: CC1(C)c2ccccc2C(O)(c2ccccc2-c2cccc(Cl)c2)c2ccccc21 ; CC1(C)c2ccccc2C2(c3ccccc3-c3cc(Cl)ccc32)c2ccccc21]

A target compound (28.6 g, 60%) corresponding to a structural isomer of Core 1 was obtained in the same manner as in Step 2 of [Preparation Example 1].

[LCMS]: 392

### [Preparation Example 3] Synthesis of Core 3

### <Step 1> Synthesis of 9-(2′-bromo-[1,1′-biphenyl]-2-yl)-10,10-dimethyl-9,10-dihydroanthracen-9-ol

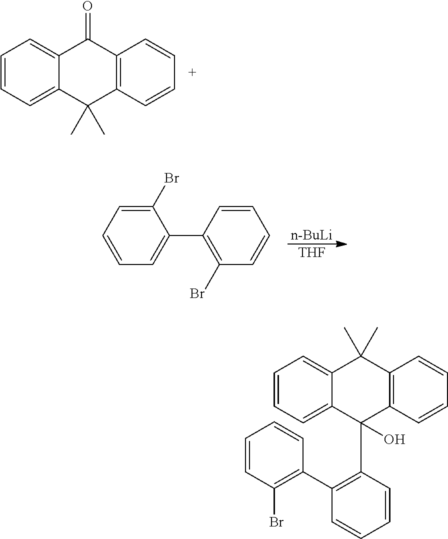

A target compound (42.3 g 58%) corresponding to a structural isomer of Core 1 was obtained in the same manner as in Step 1 of [Preparation Example 1] except that 2,2′-dibromo-1,1′-biphenyl was used as the reaction material.

[LCMS]: 455

### <Step 2> Synthesis of 4′-bromo-10,10-dimethyl-10H-spiro[anthracene-9,9′-fluorene]

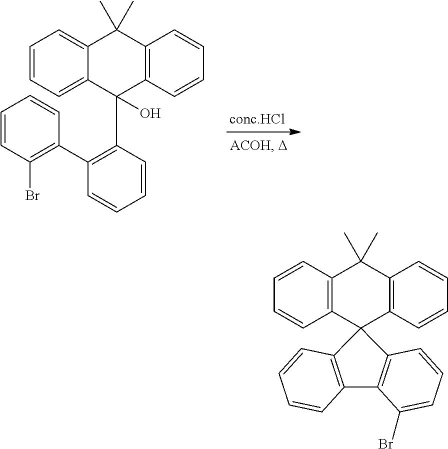 [SMILES: CC1(C)c2ccccc2C(O)(c2ccccc2-c2ccccc2Br)c2ccccc21 ; CC1(C)c2ccccc2C2(c3ccccc3-c3c(Br)cccc32)c2ccccc21]

A target compound (38.3 g, 95%) corresponding to a structural isomer of Core 1 was obtained in the same manner as in Step 2 of [Preparation Example 1].

¹H-NMR (in CDCl₃): δ 8.68 (d, 1H), 7.60 (d, 2H), 7.48 (d, 1H), 7.37 (t, 1H), 7.28 (m, 3H), 6.97 (t, 1H), 6.76 (m, 4H), 6.29 (d, 2H), 1.97 (s, 6H)

[LCMS]: 437

### [Preparation Example 4] Synthesis of Core 4

### <Step 1> Synthesis of 9-(4′-chloro-[1,1′-biphenyl]-2-yl)-10,10-diphenyl-9,10-dihydroanthracen-9-ol

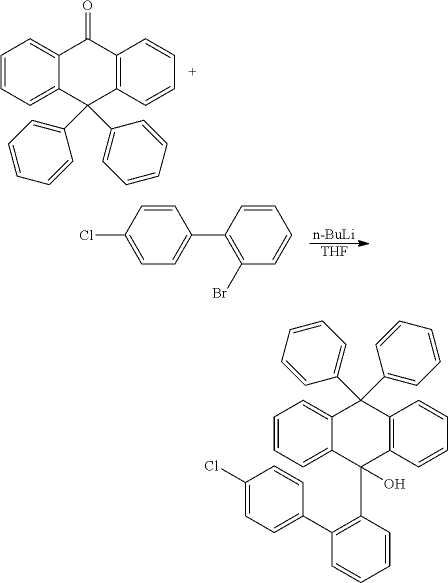

THF (500 mL) was added to 2-bromo-4′-chloro-1,1′-biphenyl (50 g, 0.19 mol). Then, the temperature of the reaction solution was lowered to −78° C., and a 1.6 M n-BuLi solution (128 mL, 0.21 mol) was slowly added dropwise to the reaction solution. After stirring the result for 1 hour at the same temperature, 10,10-diphenylanthracen-9(10H)-one (45.7 g, 0.21 mol) dissolved in THF (500 mL) was slowly added to the reaction solution, and the result was stirred for 1 hour at the same temperature, and then further stirred for 24 hours at room temperature. Then, purified water (500 mL) was introduced to the reaction solution to terminate the reaction, and then the result was extracted with E.A (2.0 L) and washed with distilled water. After that, the obtained organic layer was dried with anhydrous MgSO₄, vacuum distilled, and then purified using silica gel column chromatography to obtain a target compound (62.0 g, yield 62%).

[LCMS]: 535

### <Step 2> Synthesis of 2′-chloro-10,10-diphenyl-10H-spiro[anthracene-9,9′-fluorene]

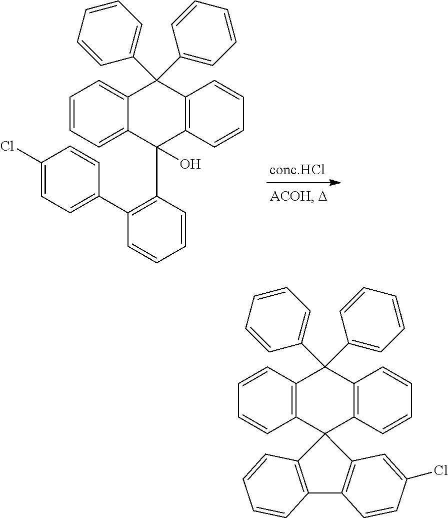 [SMILES: OC1(c2ccccc2-c2ccc(Cl)cc2)c2ccccc2C(c2ccccc2)(c2ccccc2)c2ccccc21 ; Clc1ccc2c(c1)C1(c3ccccc3-2)c2ccccc2C(c2ccccc2)(c2ccccc2)c2ccccc21]

Conc. HCl (90 mL) and AcOH (900 mL) were added to 9-(4′-chloro-[1,1′-biphenyl]-2-yl)-10,10-diphenyl-9,10-dihydroanthracen-9-ol (62.0 g, 0.12 mol). The reaction solution was heated under reflux for 2 hours at 100° C. The temperature was lowered to room temperature, and after introducing purified water (500 mL) to the reaction solution to terminate the reaction, the produced solids were vacuum filtered and dried using warm air to obtain a target compound (57.5 g, yield 96%).

[LCMS]: 517

### [Preparation Example 5] Synthesis of 4,4,5,5-tetramethyl-2-(spiro[fluorene-9,9′-xanthen]-2-yl)-1,3,2-dioxaborolane

### <Step 1> Synthesis of 9-(3′-chloro-[1,1′-biphenyl]-2-yl)-10,10-diphenyl-9,10-dihydroanthracen-9-ol

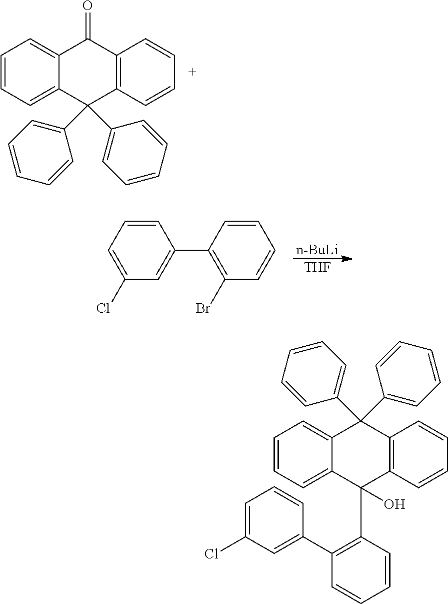

A target compound (66.0 g, 66%) corresponding to a structural isomer of Core 4 was obtained in the same manner as in Step 1 of [Preparation Example 4].

[LCMS]: 535

### <Step 2> Synthesis of 3′-chloro-10,10-diphenyl-10H-spiro[anthracene-9,9′-fluorene]

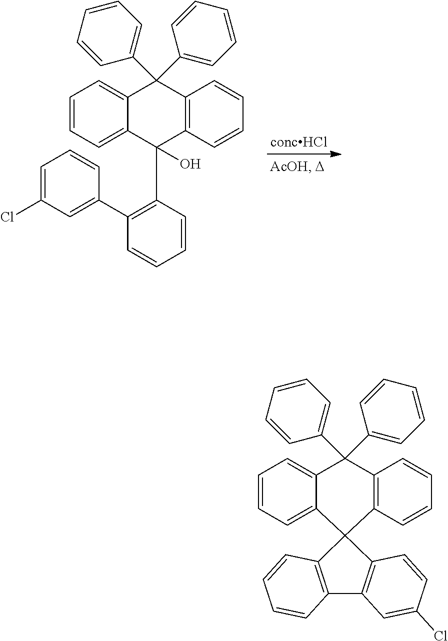 [SMILES: OC1(c2ccccc2-c2cccc(Cl)c2)c2ccccc2C(c2ccccc2)(c2ccccc2)c2ccccc21 ; Clc1ccc2c(c1)-c1ccccc1C21c2ccccc2C(c2ccccc2)(c2ccccc2)c2ccccc21]

A target compound (28.1 g, 44%) corresponding to a structural isomer of Core 4 was obtained in the same manner as in Step 2 of [Preparation Example 4].

[LCMS]: 517

### [Preparation Example 6] Synthesis of Core 6

### <Step 1> Synthesis of 9-(2′-bromo-[1,1′-biphenyl]-2-yl)-10,10-diphenyl-9,10-dihydroanthracen-9-ol

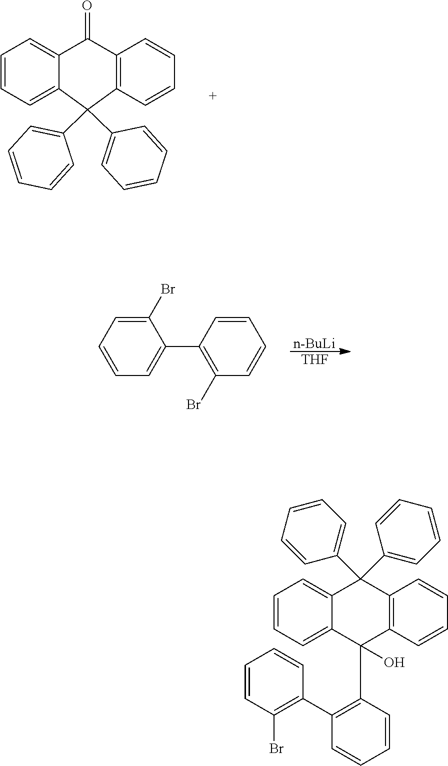

A target compound (49.2 g, 53%) corresponding to a structural isomer of Core 4 was obtained in the same manner as in Step 1 of [Preparation Example 4] except that 2,2′-dibromo-1,1′-biphenyl was used as the reaction material.

[LCMS]: 579

### <Step 2> Synthesis of 4′-bromo-10,10-dimethyl-10H-spiro[anthracene-9,9′-fluorene]

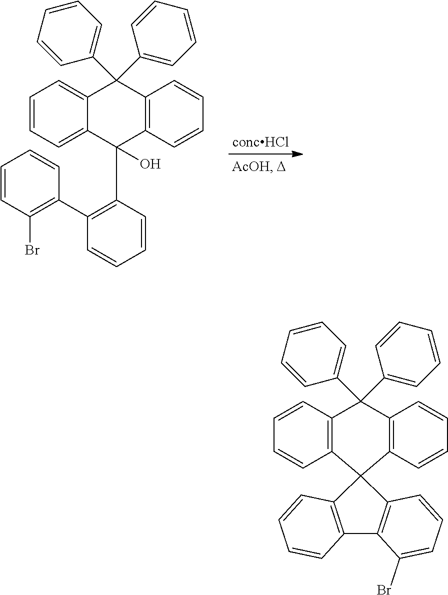 [SMILES: OC1(c2ccccc2-c2ccccc2Br)c2ccccc2C(c2ccccc2)(c2ccccc2)c2ccccc21 ; Brc1cccc2c1-c1ccccc1C21c2ccccc2C(c2ccccc2)(c2ccccc2)c2ccccc21]

A target compound (43.7 g, 92%) corresponding to a structural isomer of Core 4 was obtained in the same manner as in Step 2 of [Preparation Example 4].

[LCMS]: 561

### [Preparation Example 7] Synthesis of 2-(10,10-dimethyl-10H-spiro[anthracene-9,9′-fluoren]-2′-yl)-4,4,5,5-tetramethyl-1,3,2-dioxaborolane

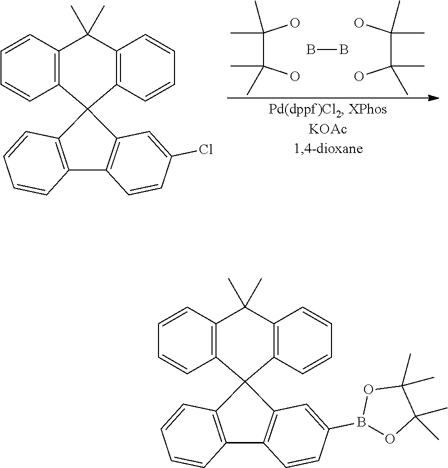

Dioxane (500 mL) was added to 2′-chloro-10,10-dimethyl-10H-spiro[anthracene-9,9′-fluorene] (20.0 g, 50.9 mmol) synthesized in [Preparation Example 1] and 4,4,4′,4′,5,5,5′,5′-octamethyl-2,2′-bi(1,3,2-dioxaborolane) (15.5 g, 61.1 mmol). Then, Pd(dppf)Cl₂ (2.1 g, 2.6 mmol), XPhos (2.4 g, 5.1 mmol) and KOAc (15.0 g, 153 mmol) were added thereto, and the result was heated under reflux for 8 hours at 130° C. Then, the temperature was lowered to room temperature, and an aqueous ammonium chloride solution (500 mL) was introduced to the reaction solution to terminate the reaction, and then the result was extracted with E.A (1.0 L) and washed with distilled water. After that, the obtained organic layer was dried with anhydrous MgSO₄, vacuum distilled, and then purified using silica gel column chromatography to obtain a target compound (21.0 g, yield 85%).

¹H-NMR (in CDCl₃): δ 7.92 (m, 3H), 7.60 (d, 2H), 7.34 (s, 1H), 7.30 (t, 1H), 7.18 (dt, 2H), 7.11 (t, 1H), 6.84 (m, 3H), 6.29 (dd, 2H), 1.92 (dd, 6H), 1.34 (s, 12H)

[LCMS]: 484

### [Preparation Example 8] Synthesis of 2-(10,10-dimethyl-10H-spiro[anthracene-9,9′-fluoren]-3′-yl)-4,4,5,5-tetramethyl-1,3,2-dioxaborolane

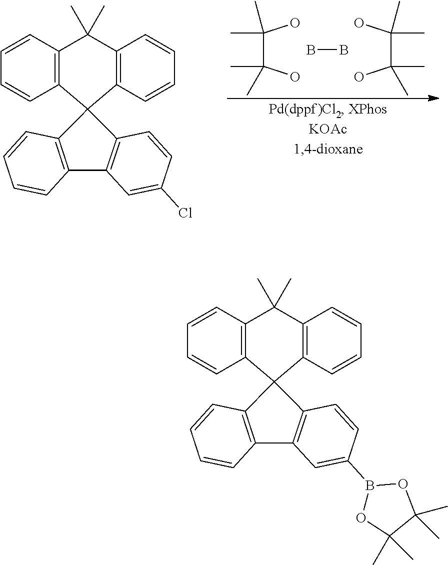

A target compound (19.2 g, yield 78%) was obtained in the same manner as in [Preparation Example 7] except that 3′-chloro-10,10-dimethyl-1 OH-spiro[anthracene-9,9′-fluorene] was used instead of 2′-chloro-10,10-dimethyl-1 OH-spiro[anthracene-9,9′-fluorene].

[LCMS]: 484

### [Preparation Example 9] Synthesis of 2-(10,10-dimethyl-10H-spiro[anthracene-9,9′-fluoren]-4′-yl)-4,4,5,5-tetramethyl-1,3,2-dioxaborolane

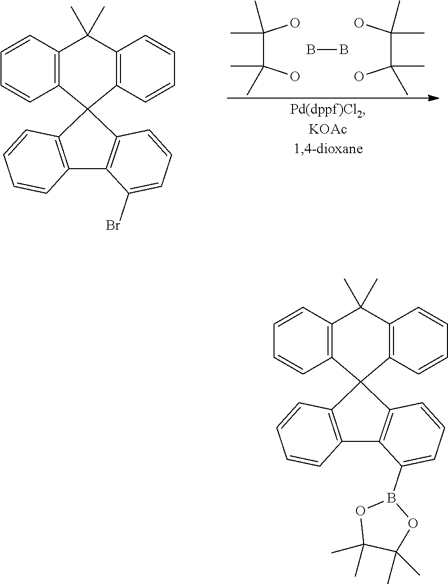

Dioxane (500 mL) was added to 4′-bromo-10,10-dimethyl-10H-spiro[anthracene-9,9′-fluorene] (15.0 g, 34.3 mmol) synthesized in [Preparation Example 3] and 4,4,4′,4′,5,5,5′,5′-octamethyl-2,2′-bi(1,3,2-dioxaborolane) (10.5 g, 41.2 mmol). Then, Pd(dppf)Cl₂ (1.5 g 1.3 mmol) and KOAc (10.1 g, 103 mmol) were added thereto, and the result was heated under reflux for 3 hours at 130° C. Then, the temperature was lowered to room temperature, and an aqueous ammonium chloride solution (500 mL) was introduced to the reaction solution to terminate the reaction, and then the result was extracted with E.A (1.0 L) and washed with distilled water. After that, the obtained organic layer was dried with anhydrous MgSO₄, vacuum distilled, and then purified using silica gel column chromatography to obtain a target compound (9.1 g, yield 55%).

¹H-NMR (in CDCl₃): δ 8.76 (d, 2H), 7.92 (dd, 1H), 7.58 (dd, 2H), 7.27 (dt, 1H), 7.09 (m, 4H), 6.93 (dd, 1H), 6.79 (m, 3H), 6.26 (dd, 2H), 1.87 (dd, 6H), 1.48 (s, 12H)

[LCMS]: 484

### [Preparation Example 10] Synthesis of 2-(10,10-Biphenyl-10H-spiro[anthracene-9,9′-fluoren]-2′-yl)-4,4,5,5-tetramethyl-1,3,2-dioxaborolane

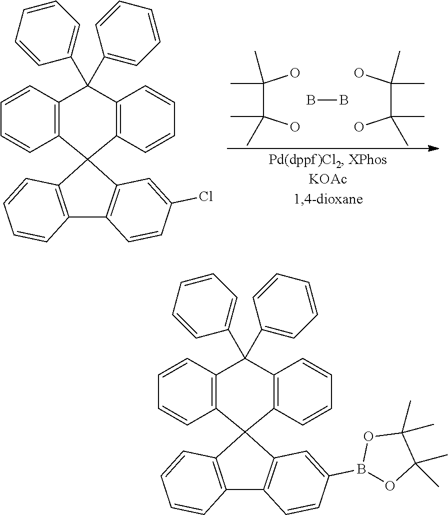

A target compound (16.8 g, yield 82%) was obtained in the same manner as in [Preparation Example 7] except that 2′-chloro-10,10-diphenyl-10H-spiro[anthracene-9,9′-fluorene] was used.

[LCMS]: 608

### [Preparation Example 11] Synthesis of 2-(10,10-diphenyl-10H-spiro[anthracene-9,9′-fluoren]-3′-yl)-4,4,5,5-tetramethyl-1,3,2-dioxaborolane

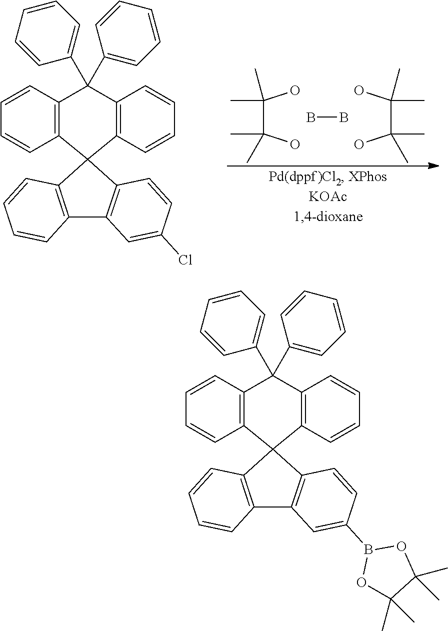

A target compound (19.4 g, yield 84%) was obtained in the same manner as in [Preparation Example 7] except that 3′-chloro-10,10-diphenyl-10H-spiro[anthracene-9,9′-fluorene].

[LCMS]: 608

### [Preparation Example 12] Synthesis of 2-(10,10-diphenyl-10H-spiro[anthracene-9,9′-fluoren]-4′-yl)-4,4,5,5-tetramethyl-1,3,2-dioxaborolane

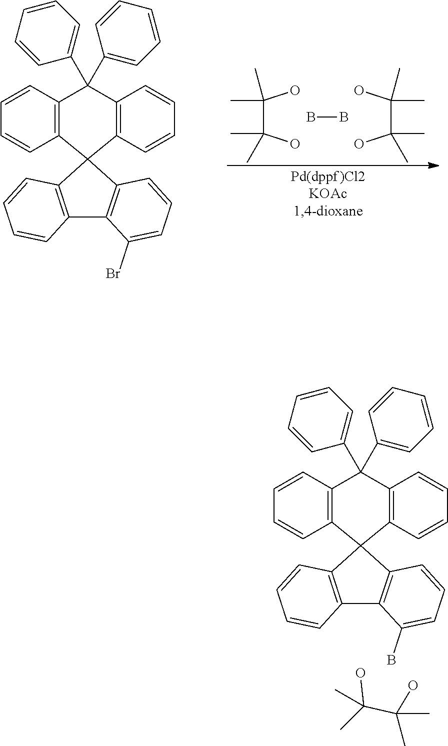

A target compound (10.2 g, yield 52%) was obtained in the same manner as in [Preparation Example 9] except that 4′-bromo-10,10-diphenyl-10H-spiro[anthracene-9,9′-fluorene] was used

[LCMS]: 608

### [Synthesis Example 1] Synthesis of Compound 2

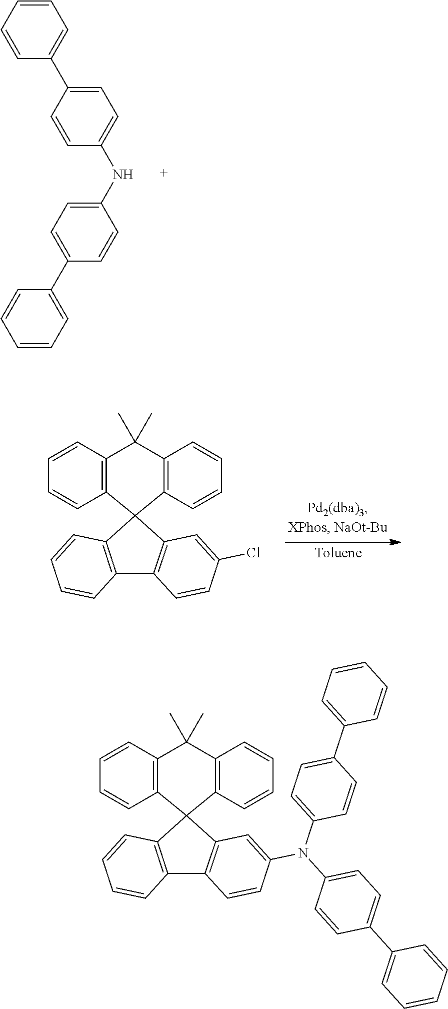 [SMILES: c1ccc(-c2ccc(Nc3ccc(-c4ccccc4)cc3)cc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccc(-c5ccccc5)cc4)c4ccc(-c5ccccc5)cc4)cc32)c2ccccc21]

Toluene (100 mL) was added to Preparation Example 1 (8.1 g, 20.6 mmol) and di([1,1′-biphenyl]-4-yl)amine (6.0 g, 18.7 mmol). Pd₂(dba)₃ (0.91 g, 1.0 mmol), XPhos (0.91 g, 1.9 mmol) and NaOt-Bu (3.6 g, 37.4 mmol) were introduced to the reaction solution, and the result was heated under reflux for 5 hours at 120° C. The temperature was lowered to room temperature, and purified water (300 mL) was introduced to the reaction solution to terminate the reaction. The mixture solution was extracted with E.A (500 mL) and then washed with distilled water. The obtained organic layer was dried with anhydrous MgSO₄, vacuum distilled, and purified using silica gel column chromatography to obtain a target compound (8.6 g, yield 68%).

[LCMS]: 677

### [Synthesis Example 2] Synthesis of Compound 4

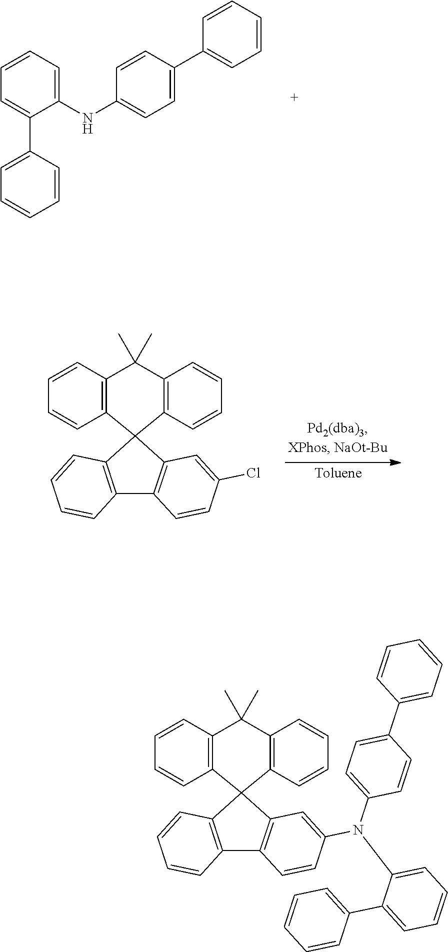 [SMILES: c1ccc(-c2ccc(Nc3ccccc3-c3ccccc3)cc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccc(-c5ccccc5)cc4)c4ccccc4-c4ccccc4)cc32)c2ccccc21]

A target compound (5.5 g, yield 72%) was obtained in the carne manner as in [Synthesis Example 1] except that 2′-chloro-10,10-dimethyl-10H-spiro[anthracene-9,9′-fluorene] of Preparation Example 1 and N-([1,1′-biphenyl]-4-yl)-[1,1′-biphenyl]-2-amine were used.

[LCMS]: 677

### [Synthesis Example 3] Synthesis of Compound 6

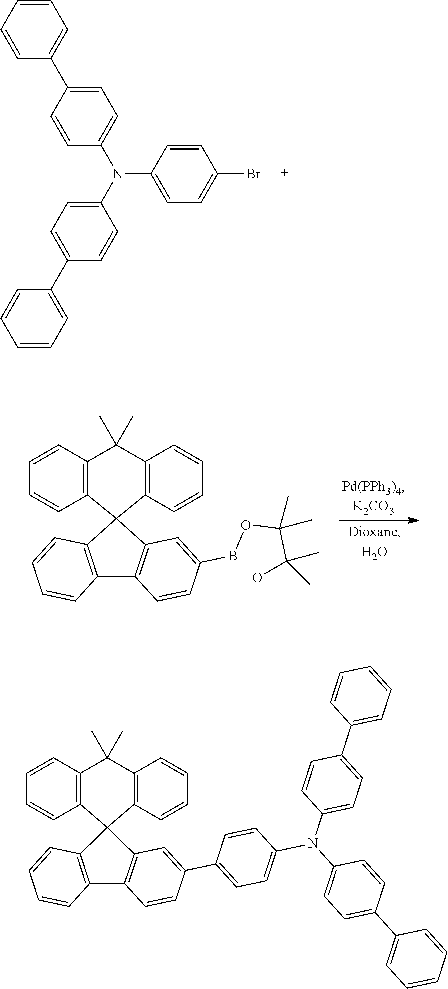

Dioxane (100 mL) and H₂O (25 mL) were added to Preparation Example 7 (8 g, 16.6 mmol) synthesized above and 2-chloro-4,6-diphenyl-1,3,5-triazine (8.7 g, 18.3 mmol) Pd(PPh₃)₄ (1.0 g, 0.9 mmol) and K₂CO₃ (6.9 g, 49.8 mmol) were added thereto, and the result was heated under reflux for 6 hours at 120° C. The temperature was lowered to room temperature, and purified water (300 mL) was introduced to the reaction solution to terminate the reaction. The mixture solution was extracted with E.A (1.0 L) and then washed with distilled water. The obtained organic layer was dried with anhydrous MgSO₄, vacuum distilled, and purified using silica gel column chromatography to obtain a target compound (8.3 g, yield 66%).

[LCMS]: 753

### [Synthesis Example 4] Synthesis of Compound 8

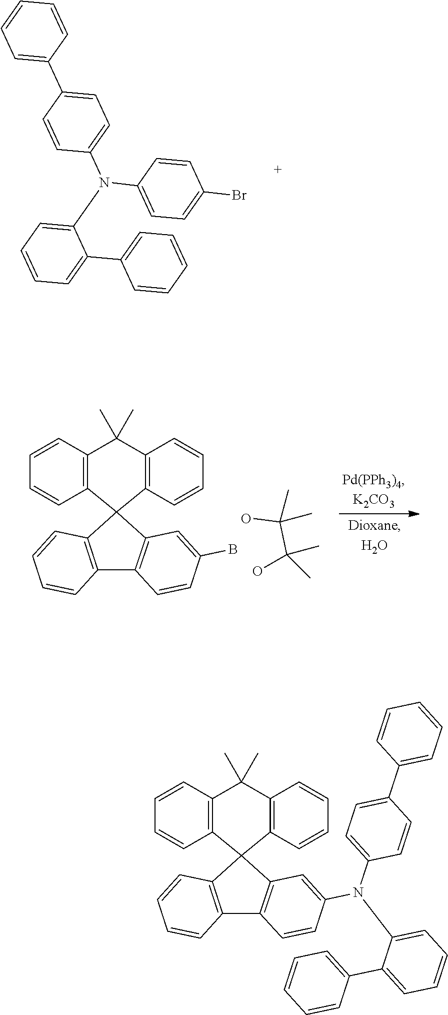

A target compound (6.0 g, yield 62%) was obtained in the same manner as in [Synthesis Example 3] except that 2-(10,10-dimethyl-10H-spiro[anthracene-9,9′-fluoren]-2′5-tetramethyl-1,3,2-dioxaborolane of Preparation Example 7 and N-([1,1′-biphenyl]-4-yl)-N-(4-bromophenyl)-[1,1′-biphenyl]-2-amine were used.

[LCMS]: 753

### [Synthesis Example 5] Synthesis of Compound 12

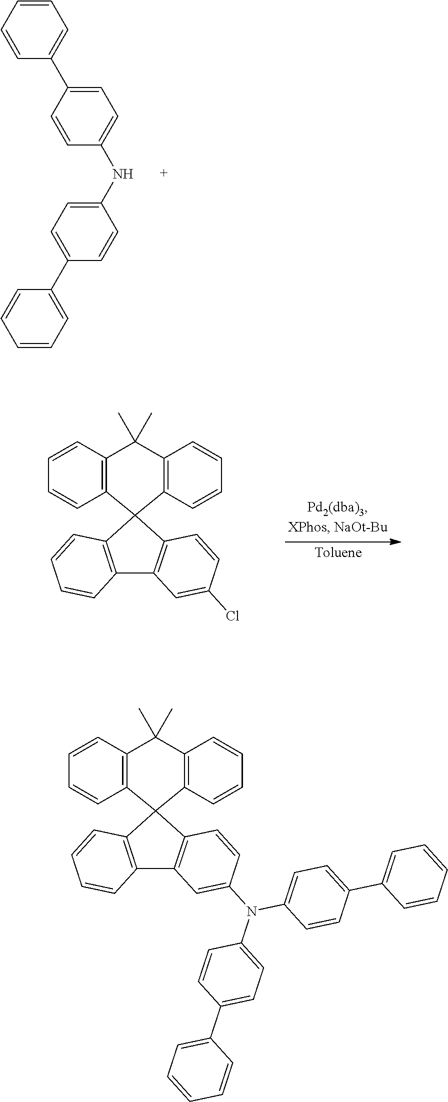 [SMILES: c1ccc(-c2ccc(Nc3ccc(-c4ccccc4)cc3)cc2)cc1 ; CC1(C)c2ccccc2C2(c3ccccc3-c3cc(Cl)ccc32)c2ccccc21 ; CC1(C)c2ccccc2C2(c3ccccc3-c3cc(N(c4ccc(-c5ccccc5)cc4)c4ccc(-c5ccccc5)cc4)ccc32)c2ccccc21]

A target compound (6.5 g, yield 66%) was obtained in the same manner as in [Synthesis Example 1] except that 3′-chloro-10,10-dimethyl-10H-spiro[anthracene-9,9′-fluorene] of Preparation Example 2 and di([1,1′-biphenyl]-4-yl)amine were used.

[LCMS]: 677

### [Synthesis Example 6] Synthesis of Compound 14

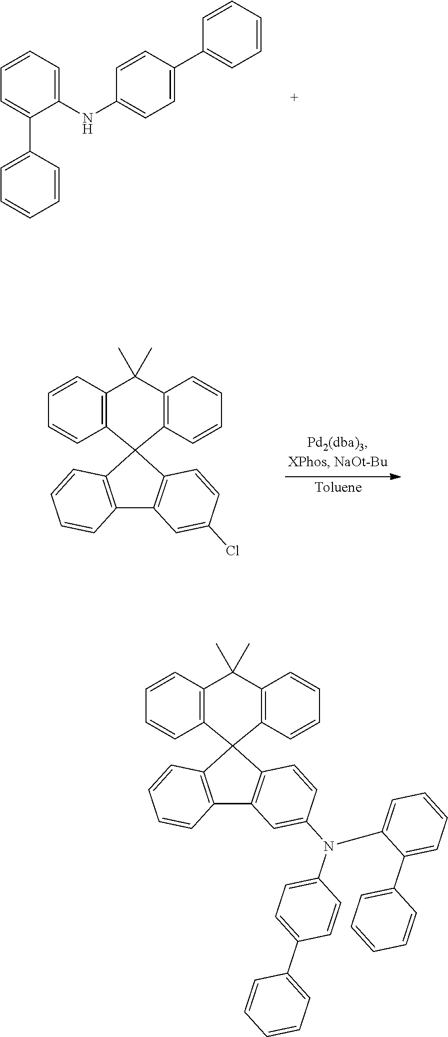 [SMILES: c1ccc(-c2ccc(Nc3ccccc3-c3ccccc3)cc2)cc1 ; CC1(C)c2ccccc2C2(c3ccccc3-c3cc(Cl)ccc32)c2ccccc21 ; CC1(C)c2ccccc2C2(c3ccccc3-c3cc(N(c4ccc(-c5ccccc5)cc4)c4ccccc4-c4ccccc4)ccc32)c2ccccc21]

A target compound (5.0 g, yield 62%) was obtained in the same manner as in [Synthesis Example 1] except that 3′-chloro-10,10-dimethyl-10H-spiro[anthracene-9,9′-fluorene] of Preparation Example 2 and N-([1,1′-biphenyl]-4-yl)-[1,1′-biphenyl]-2-amine were used.

[LCMS]: 677

### [Synthesis Example 7] Synthesis of Compound 16

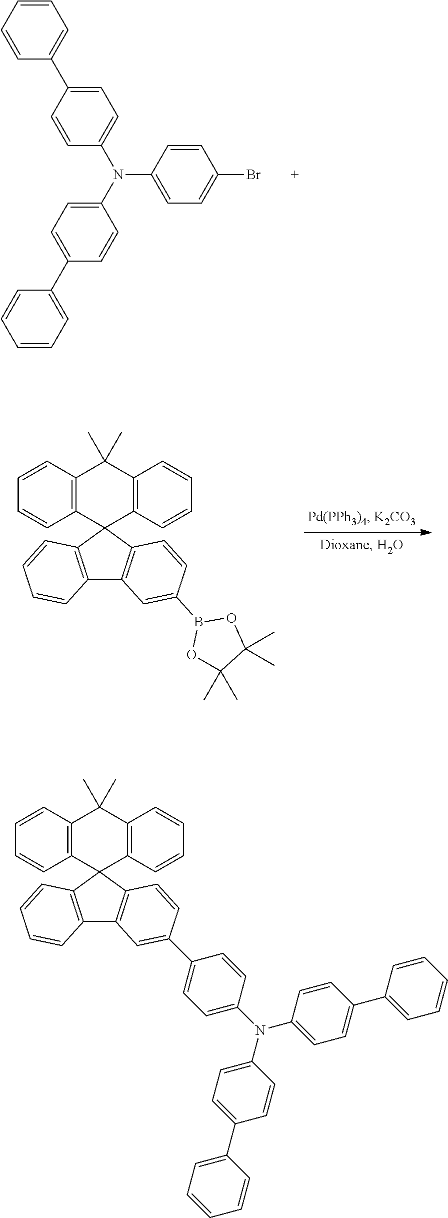 [SMILES: Brc1ccc(N(c2ccc(-c3ccccc3)cc2)c2ccc(-c3ccccc3)cc2)cc1 ; CC1(C)c2ccccc2C2(c3ccccc3-c3cc(B4OC(C)(C)C(C)(C)O4)ccc32)c2ccccc21 ; CC1(C)c2ccccc2C2(c3ccccc3-c3cc(-c4ccc(N(c5ccc(-c6ccccc6)cc5)c5ccc(-c6ccccc6)cc5)cc4)ccc32)c2ccccc21]

A target compound (4.5 g, yield 58%) was obtained in the same manner as in [Synthesis Example 3] except that 2-(10,10-dimethyl-10H-spiro[anthracene-9,9′-fluoren]-3′-yl)-4,4,5,5-tetramethyl-1,3,2-dioxaborolane of Preparation Example 8 and N-([1,1′-biphenyl]-4 yl)-N-(4-bromophenyl)-[1,1′-biphenyl]-4-amine were used.

[LCMS]: 753

### [Synthesis Example 8] Synthesis of Compound 19

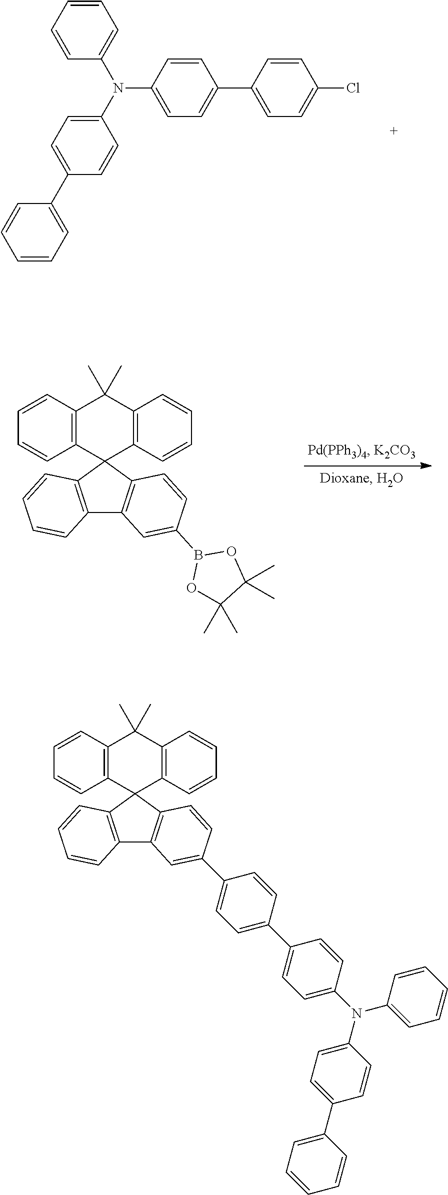 [SMILES: Clc1ccc(-c2ccc(N(c3ccccc3)c3ccc(-c4ccccc4)cc3)cc2)cc1 ; CC1(C)c2ccccc2C2(c3ccccc3-c3cc(B4OC(C)(C)C(C)(C)O4)ccc32)c2ccccc21 ; CC1(C)c2ccccc2C2(c3ccccc3-c3cc(-c4ccc(-c5ccc(N(c6ccccc6)c6ccc(-c7ccccc7)cc6)cc5)cc4)ccc32)c2ccccc21]

A target compound (7.4 g, yield 69%) was obtained in the same manner as in [Synthesis Example 3] except that 2-(10,10-dimethyl-10H-spiro[anthracene-9,9′-fluoren]-3′-yl)-4,4,5,5-tetramethyl-1,3,2-dioxaborolane of Preparation Example 8 and N-([1,1′-biphenyl]-4-yl)-4′-chloro-N-phenyl-[1,1′-biphenyl]-4-amine were used.

[LCMS]: 753

### [Synthesis Example 9] Synthesis of Compound 22

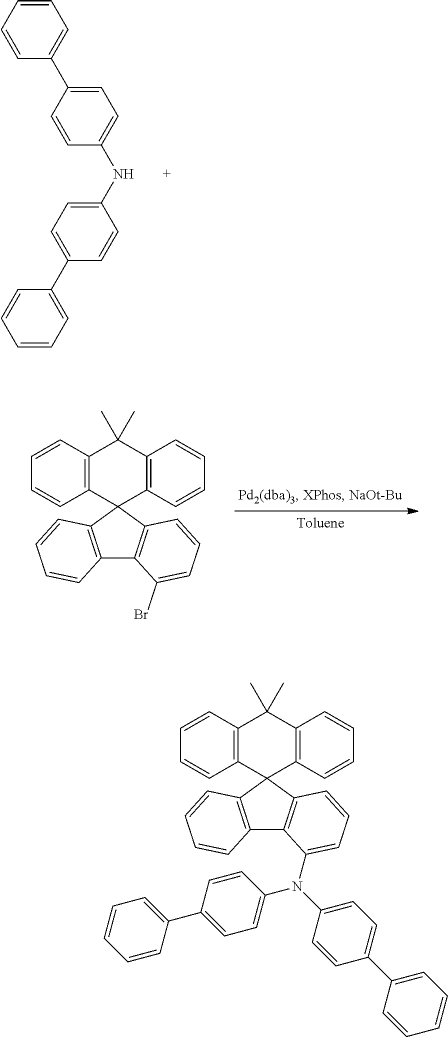 [SMILES: c1ccc(-c2ccc(Nc3ccc(-c4ccccc4)cc3)cc2)cc1 ; CC1(C)c2ccccc2C2(c3ccccc3-c3c(Br)cccc32)c2ccccc21 ; CC1(C)c2ccccc2C2(c3ccccc3-c3c(N(c4ccc(-c5ccccc5)cc4)c4ccc(-c5ccccc5)cc4)cccc32)c2ccccc21]

A target compound (5.8 g, yield 55%) was obtained in the same manner as in [Synthesis Example 1] except that 4′-bromo-10,10-dimethyl-10H-spiro[anthracene-9,9′-fluorene] of Preparation Example 3 and di([1,1′-biphenyl]-4-yl)amine were used.

[LCMS]: 677

### [Synthesis Example 10] Synthesis of Compound 24

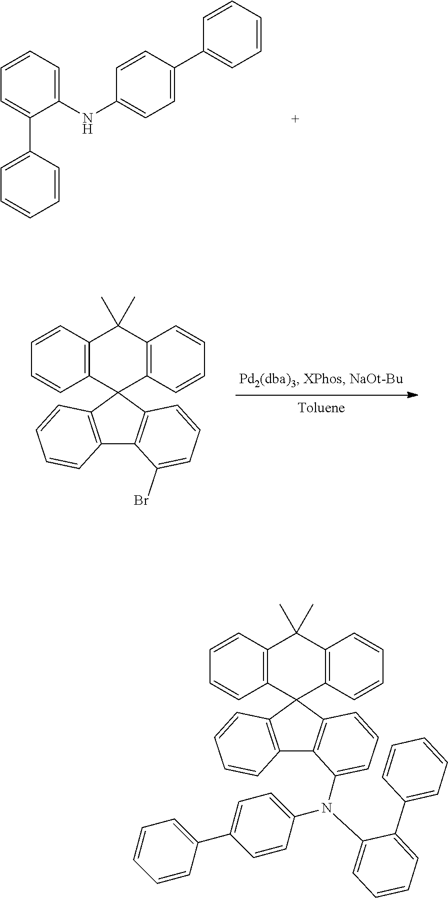 [SMILES: c1ccc(-c2ccc(Nc3ccccc3-c3ccccc3)cc2)cc1 ; CC1(C)c2ccccc2C2(c3ccccc3-c3c(Br)cccc32)c2ccccc21 ; CC1(C)c2ccccc2C2(c3ccccc3-c3c(N(c4ccc(-c5ccccc5)cc4)c4ccccc4-c4ccccc4)cccc32)c2ccccc21]

A target compound (6.5 g, yield 67%) was obtained in the same manner as in [Synthesis Example 1] except that 4′-bromo-10,10-dimethyl-10H-spiro[anthracene-9,9′-fluorene] of Preparation Example 3 and N-([1,1′-biphenyl]-4-yl)-[1,1′-biphenyl]-2-amine were used.

[LCMS]: 677

### [Synthesis Example 11] Synthesis of Compound 26

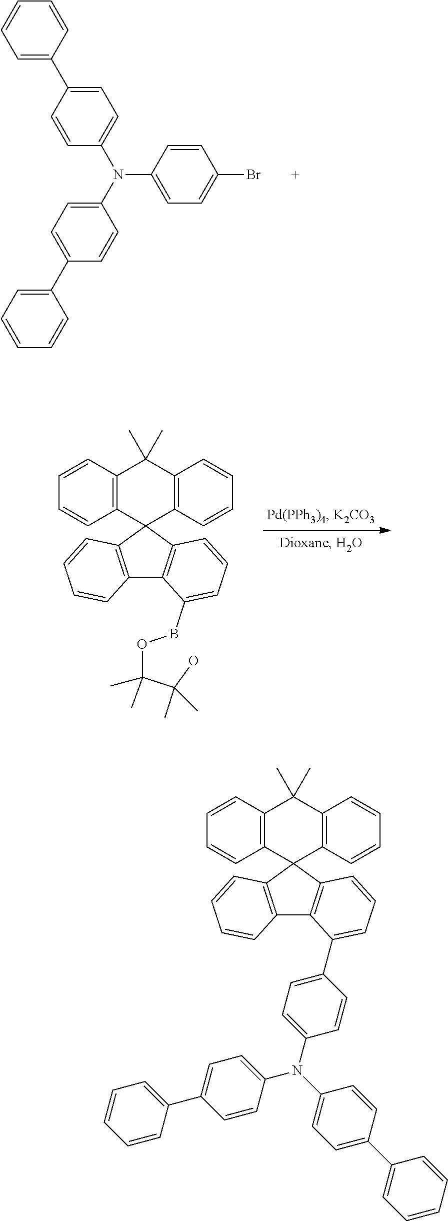

A target compound (3.8 g, yield 60%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 9 and N-([1,1′-biphenyl]-4-yl)-N-(4-bromophenyl)-[1,1′-biphenyl]-4-amuse were used.

[LCMS]: 753

### [Synthesis Example 12] Synthesis of Compound 30

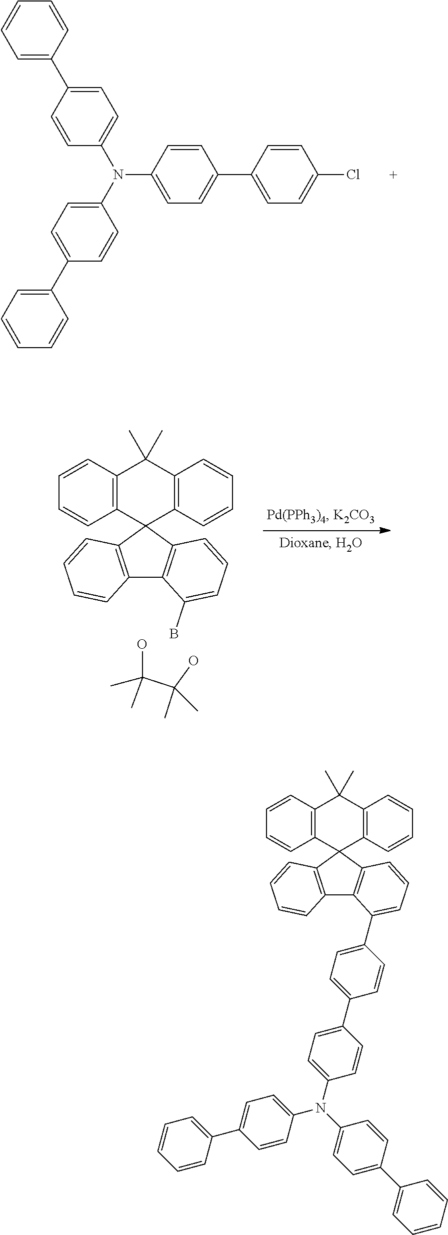

A target compound (4.4 g, yield 58%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 9 and N,N-di([1,1′-biphenyl]-4-yl)-4′-chloro-[1,1′-biphenyl]-4-amine were used.

[LCMS]: 830

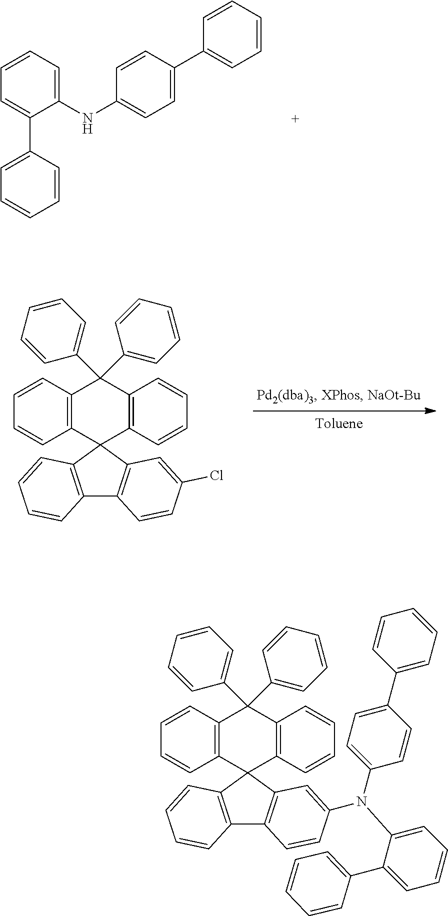 [SMILES: c1ccc(-c2ccc(Nc3ccccc3-c3ccccc3)cc2)cc1 ; Clc1ccc2c(c1)C1(c3ccccc3-2)c2ccccc2C(c2ccccc2)(c2ccccc2)c2ccccc21 ; c1ccc(-c2ccc(N(c3ccc4c(c3)C3(c5ccccc5-4)c4ccccc4C(c4ccccc4)(c4ccccc4)c4ccccc43)c3ccccc3-c3ccccc3)cc2)cc1]

### [Synthesis Example 13] Synthesis of Compound 34

A target compound (3.5 g, yield 56%) was obtained in the same manner as in [Synthesis Example 1] except that 2′-chloro-10,10-diphenyl-10H-spiro[anthracene-9,9′-fluorene] of Preparation Example 4 and N-([1,1′-biphenyl]-4-yl)-[1,1′-biphenyl]-2-amine were used.

[LCMS]: 802

### [Synthesis Example 14] Synthesis of Compound 36

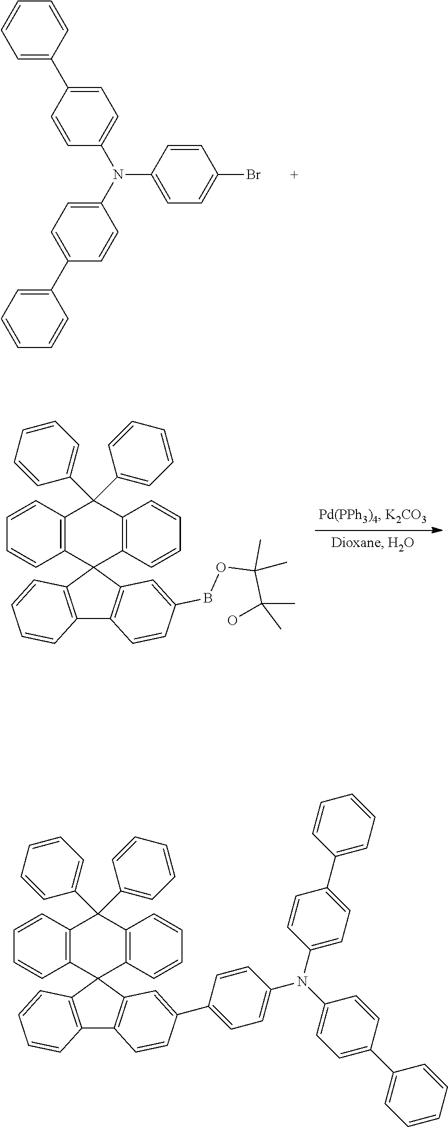

A target compound (4.7 g, yield 63%) was obtained in the same manner as in [Synthesis Example 3] except that 2-(10,10-diphenyl-10H-spiro[anthracene-9,9′-fluoren]-2′-yl)-4,4,5,5-tetramethyl-1,3,2-dioxaborolane of Preparation Example 11 and N-([1,1′-biphenyl]-4-yl)-N-(4-bromophenyl)-[1,1′-biphenyl]-4-amine were used.

[LCMS]: 878

### [Synthesis Example 15] Synthesis of Compound 42

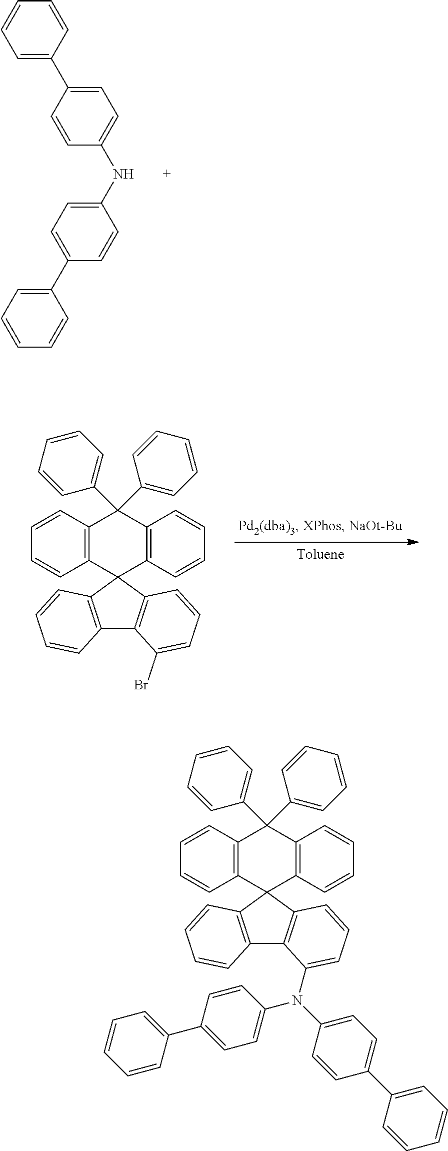 [SMILES: c1ccc(-c2ccc(Nc3ccc(-c4ccccc4)cc3)cc2)cc1 ; Brc1cccc2c1-c1ccccc1C21c2ccccc2C(c2ccccc2)(c2ccccc2)c2ccccc21 ; c1ccc(-c2ccc(N(c3ccc(-c4ccccc4)cc3)c3cccc4c3-c3ccccc3C43c4ccccc4C(c4ccccc4)(c4ccccc4)c4ccccc43)cc2)cc1]

A target compound (8.2 g, yield 70%) was obtained in the same manner as in [Synthesis Example 1] except that 4′-bromo-10,10-diphenyl-10H-spiro[anthracene-9,9′-fluorene] of Preparation Example 6 and di([1,1′-biphenyl]-4-yl)amine were used.

[LCMS]: 802

### [Synthesis Example 16] Synthesis of Compound 44

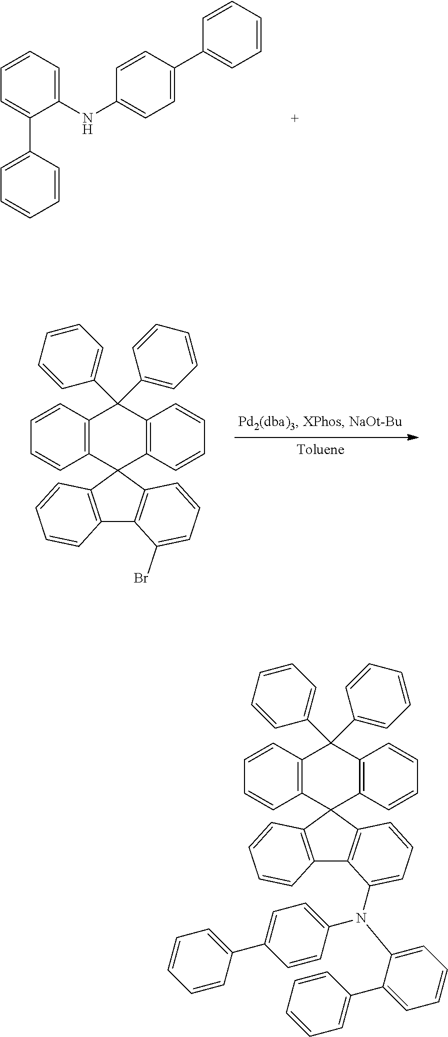 [SMILES: c1ccc(-c2ccc(Nc3ccccc3-c3ccccc3)cc2)cc1 ; Brc1cccc2c1-c1ccccc1C21c2ccccc2C(c2ccccc2)(c2ccccc2)c2ccccc21 ; c1ccc(-c2ccc(N(c3ccccc3-c3ccccc3)c3cccc4c3-c3ccccc3C43c4ccccc4C(c4ccccc4)(c4ccccc4)c4ccccc43)cc2)cc1]

A target compound (7.6 g, yield 65%) was obtained in the same manner as in [Synthesis Example 1] except that 4′-bromo-10,10-diphenyl-10H-spiro[anthracene-9,9′-fluorene] of Preparation Example 6 and N-([1,1′-biphenyl]-4-yl)-[1,1′-biphenyl]-2-amine were used.

[LCMS]: 802

### [Synthesis Example 17] Synthesis of Compound 51

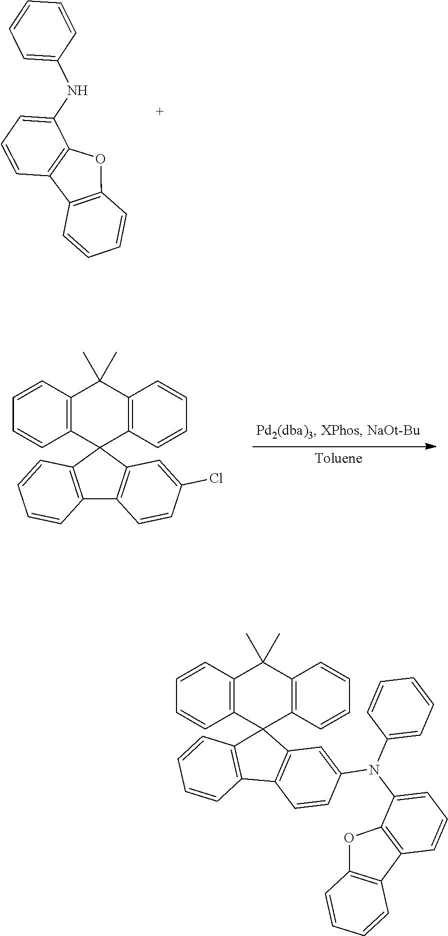 [SMILES: c1ccc(Nc2cccc3c2oc2ccccc23)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccccc4)c4cccc5c4oc4ccccc45)cc32)c2ccccc21]

A target compound (3.3 g, yield 52%) was obtained in the same manner as in [Synthesis Example 1] except that 2′-chloro-10,10-dimethyl-10H-spiro[anthracene-9,9′-fluorene] of Preparation Example 1 and N-phenyldibenzo[b,d]furan-4-amine were used.

[LCMS]: 615

### [Synthesis Example 18] Synthesis of Compound 54

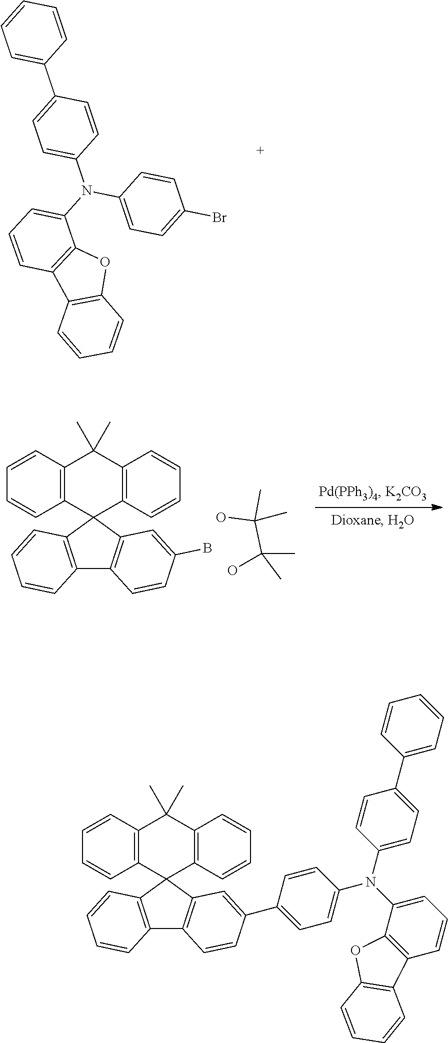

A target compound (5.0 g, yield 61%) was obtained in the same manner as in [Synthesis Example 3] except that 2-(10,10-dimethyl-10H-spiro[anthracene-9,9′-fluoren]-2′-yl)-4,4,5,5-tetramethyl-1,3,2-dioxaborolane of Preparation Example 7 and N-([1,1′-biphenyl]-4-yl)-N-(4-bromophenyl)dibenzo[b,d]furan-4-amine were used.

[LCMS]: 767

### [Synthesis Example 19] Synthesis of Compound 57

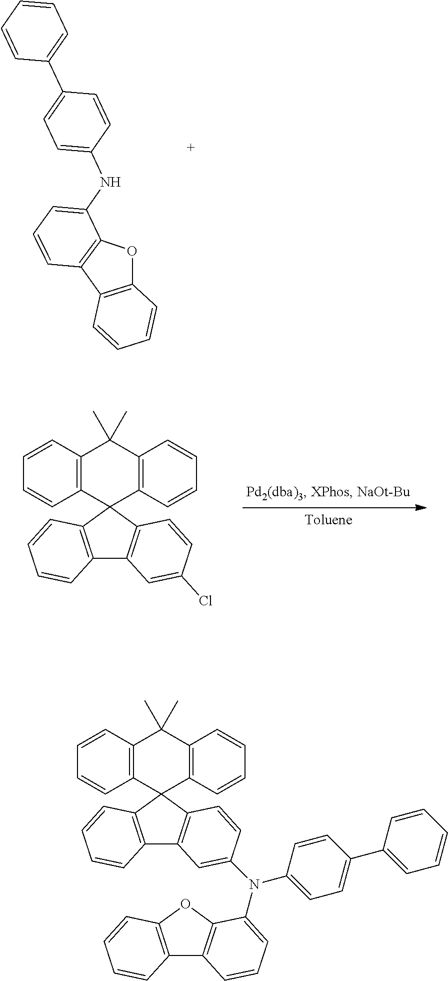 [SMILES: c1ccc(-c2ccc(Nc3cccc4c3oc3ccccc34)cc2)cc1 ; CC1(C)c2ccccc2C2(c3ccccc3-c3cc(Cl)ccc32)c2ccccc21 ; CC1(C)c2ccccc2C2(c3ccccc3-c3cc(N(c4ccc(-c5ccccc5)cc4)c4cccc5c4oc4ccccc45)ccc32)c2ccccc21]

A target compound (2.8 g, yield 55%) was obtained in the same manner as in [Synthesis Example 1] except that 3′-chloro-10,10-dimethyl-10H-spiro[anthracene-9,9′-fluorene] of Preparation Example 2 and N-([1,1′-biphenyl]-4-yl)dibenzo[b,d]furan-4-amine were used.

[LCMS]: 691

### [Synthesis Example 20] Synthesis of Compound 62

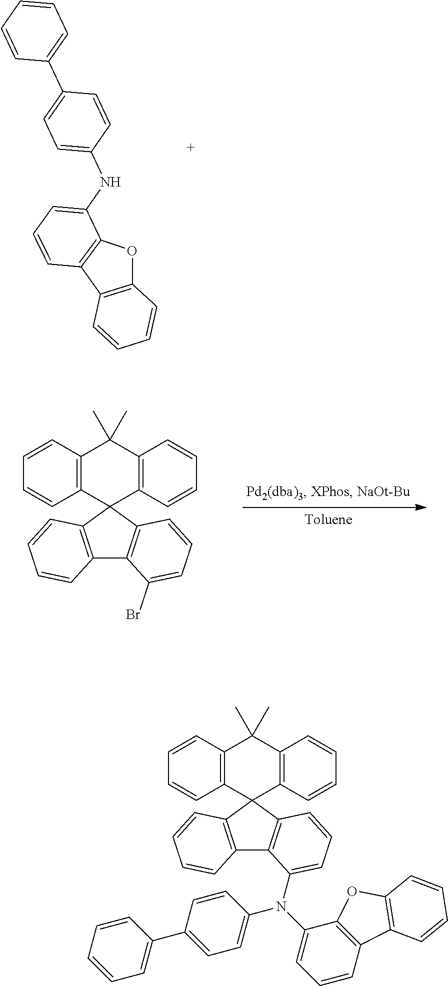 [SMILES: c1ccc(-c2ccc(Nc3cccc4c3oc3ccccc34)cc2)cc1 ; CC1(C)c2ccccc2C2(c3ccccc3-c3c(Br)cccc32)c2ccccc21 ; CC1(C)c2ccccc2C2(c3ccccc3-c3c(N(c4ccc(-c5ccccc5)cc4)c4cccc5c4oc4ccccc45)cccc32)c2ccccc21]

A target compound (6.1 g, yield 59%) was obtained in the same manner as in [Synthesis Example 1] except that 4′-bromo-10,10-dimethyl-10H-spiro[anthracene-9,9′-fluorene] of Preparation Example 3 and N-([1,1′-biphenyl]-4-yl)dibenzo[b,d]furan-4-amine were used.

[LCMS]: 691

### [Synthesis Example 21] Synthesis of Compound 67

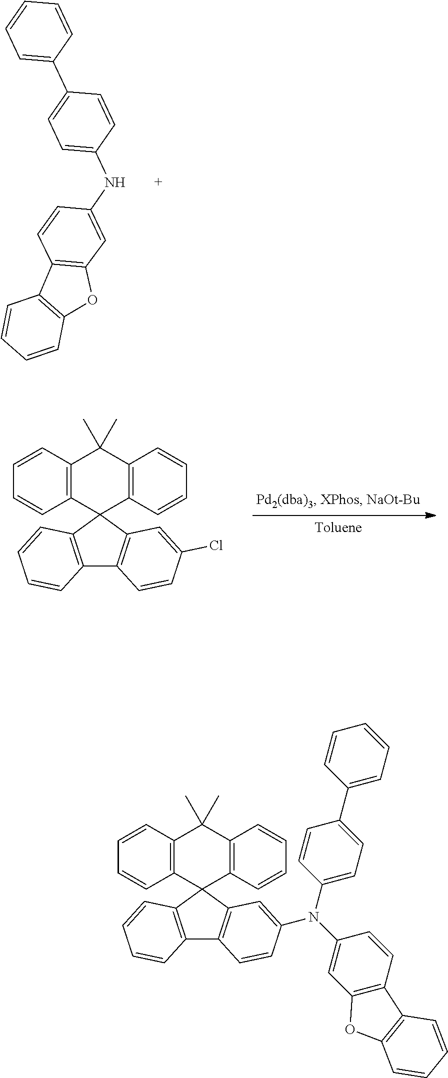 [SMILES: c1ccc(-c2ccc(Nc3ccc4c(c3)oc3ccccc34)cc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccc(-c5ccccc5)cc4)c4ccc5c(c4)oc4ccccc45)cc32)c2ccccc21]

A target compound (5.3 g, yield 50%) was obtained in the same manner as in [Synthesis Example 1] except that 2′-chloro-10,10-dimethyl-10H-spiro[anthracene-9,9′-fluorene] of Preparation Example 1 and N-([1,1′-biphenyl]-4-yl)dibenzo[b,d]furan-3-amine were used.

[LCMS]: 691

### [Synthesis Example 22] Synthesis of Compound 72

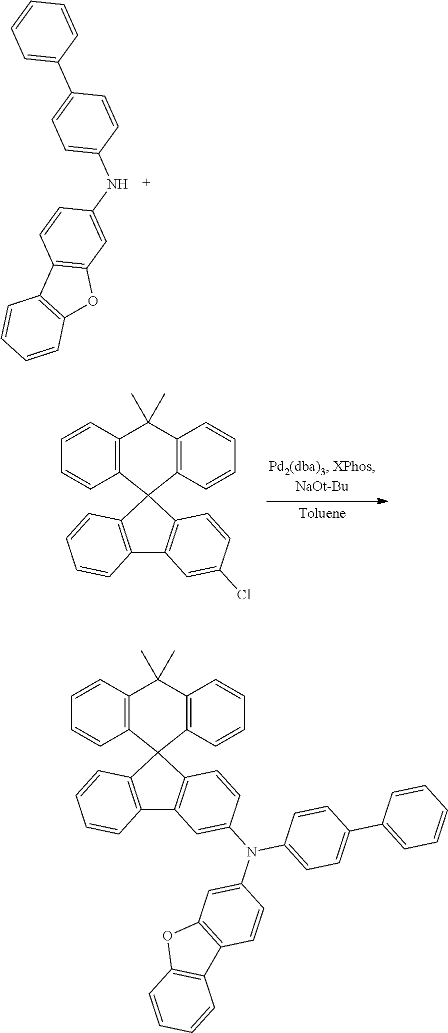 [SMILES: c1ccc(-c2ccc(Nc3ccc4c(c3)oc3ccccc34)cc2)cc1 ; CC1(C)c2ccccc2C2(c3ccccc3-c3cc(Cl)ccc32)c2ccccc21 ; CC1(C)c2ccccc2C2(c3ccccc3-c3cc(N(c4ccc(-c5ccccc5)cc4)c4ccc5c(c4)oc4ccccc45)ccc32)c2ccccc21]

A target compound (4.4 g, yield 49%) was obtained in the same manner as in [Synthesis Example 1] except that 3′-chloro-10,10-dimethyl-10H-spiro[anthracene-9,9′-fluorene] of Preparation Example 2 and N-([1,1′-biphenyl]-4-yl)dibenzo[b,d]furan-3-amine were used.

[LCMS]: 691

### [Synthesis Example 23] Synthesis of Compound 79

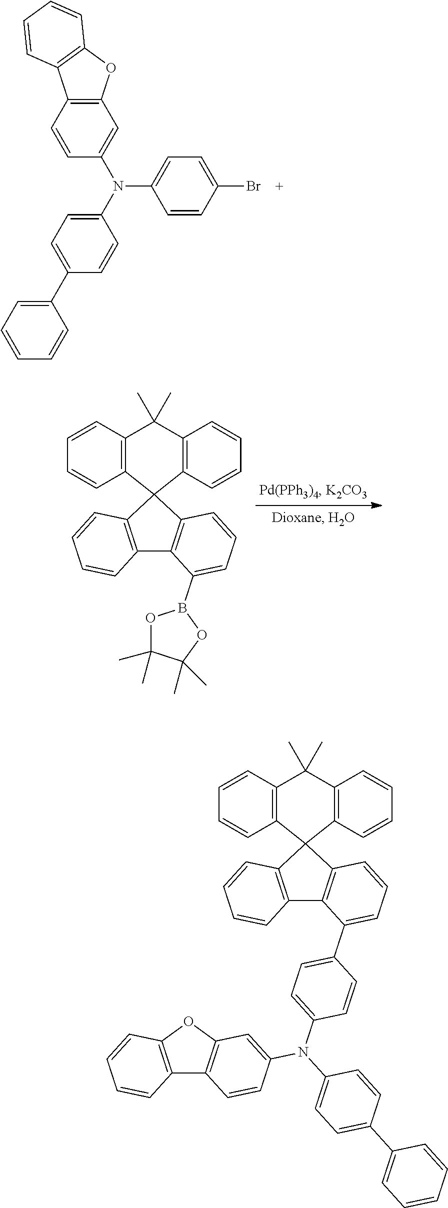 [SMILES: Brc1ccc(N(c2ccc(-c3ccccc3)cc2)c2ccc3c(c2)oc2ccccc23)cc1 ; CC1(C)c2ccccc2C2(c3ccccc3-c3c(B4OC(C)(C)C(C)(C)O4)cccc32)c2ccccc21 ; CC1(C)c2ccccc2C2(c3ccccc3-c3c(-c4ccc(N(c5ccc(-c6ccccc6)cc5)c5ccc6c(c5)oc5ccccc56)cc4)cccc32)c2ccccc21]

A target compound (6.9 g, yield 53%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 9 and N-([1,1′-biphenyl]-4-yl)-N-(4-bromophenyl)dibenzo[b,d]furan-3-amine were used.

[LCMS]: 767

### [Synthesis Example 24] Synthesis of Compound 92

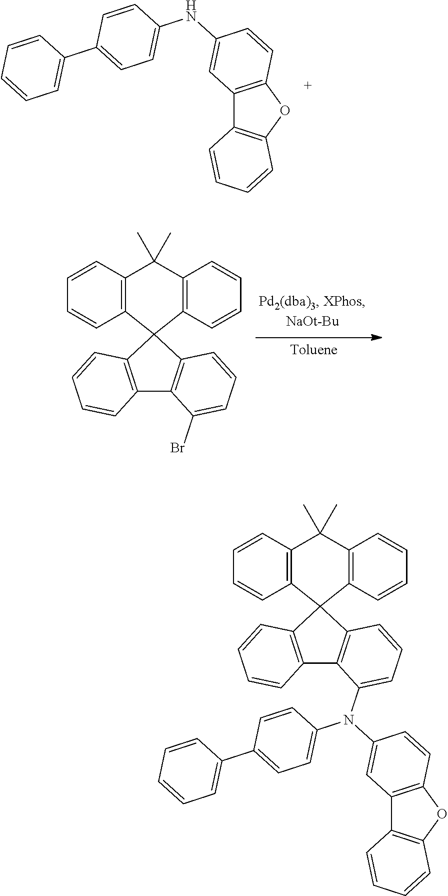 [SMILES: c1ccc(-c2ccc(Nc3ccc4oc5ccccc5c4c3)cc2)cc1 ; CC1(C)c2ccccc2C2(c3ccccc3-c3c(Br)cccc32)c2ccccc21 ; CC1(C)c2ccccc2C2(c3ccccc3-c3c(N(c4ccc(-c5ccccc5)cc4)c4ccc5oc6ccccc6c5c4)cccc32)c2ccccc21]

A target compound (5.1 g, yield 60%) was obtained in the same manner as in [Synthesis Example 1] except that 4′-bromo-10,10-dimethyl-10H-spiro[anthracene-9,9′-fluorene] of Preparation Example 3 and N-([1,1′-biphenyl]-4-yl)dibenzo[b,d]furan-2-amine were used.

[LCMS]: 691

### [Synthesis Example 25] Synthesis of Compound 97

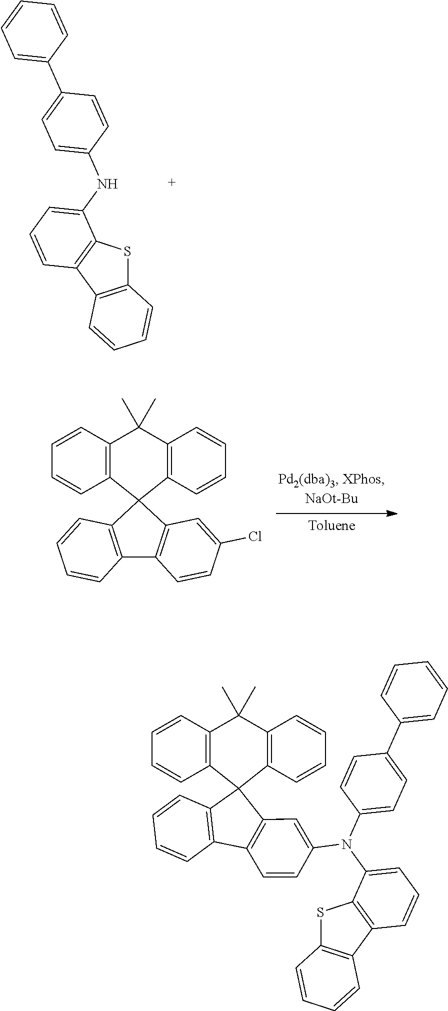 [SMILES: c1ccc(-c2ccc(Nc3cccc4c3sc3ccccc34)cc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccc(-c5ccccc5)cc4)c4cccc5c4sc4ccccc45)cc32)c2ccccc21]

A target compound (3.7 g, yield 64%) was obtained in the same manner as in [Synthesis Example 1] except that 2′-chloro-10,10-dimethyl-10H-spiro[anthracene-9,9′-fluorene] of Preparation Example 1 and N-([1,1′-biphenyl]-4-yl)dibenzo[b,d]thiophene-4-amine were used.

[LCMS]: 707

### [Synthesis Example 26] Synthesis of Compound 107

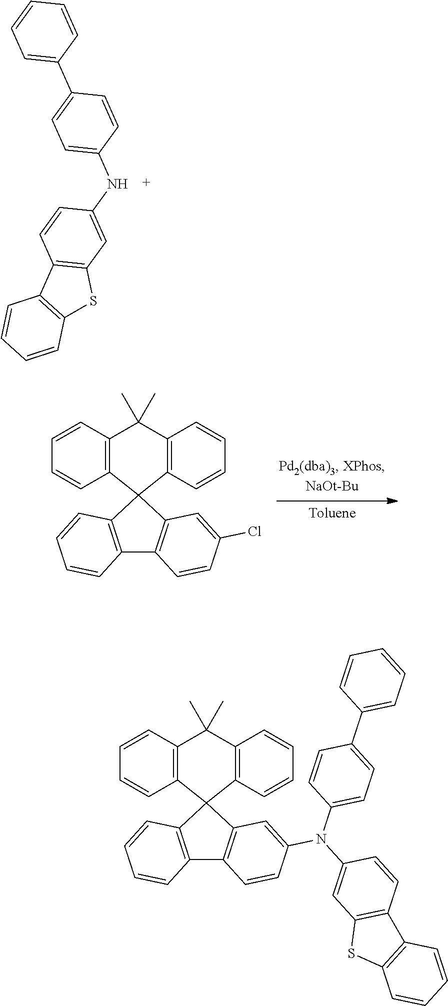 [SMILES: c1ccc(-c2ccc(Nc3ccc4c(c3)sc3ccccc34)cc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccc(-c5ccccc5)cc4)c4ccc5c(c4)sc4ccccc45)cc32)c2ccccc21]

A target compound (6.6 g, yield 53%) was obtained in the same manner as in [Synthesis Example 1] except that 2′-chloro-10,10-dimethyl-10H-spiro[anthracene-9,9′-fluorene] of Preparation Example 1 and N-([1,1′-biphenyl]-4-yl)dibenzo[b,d]thiophene-3-amine were used.

[LCMS]: 707

### [Synthesis Example 27] Synthesis of Compound 113

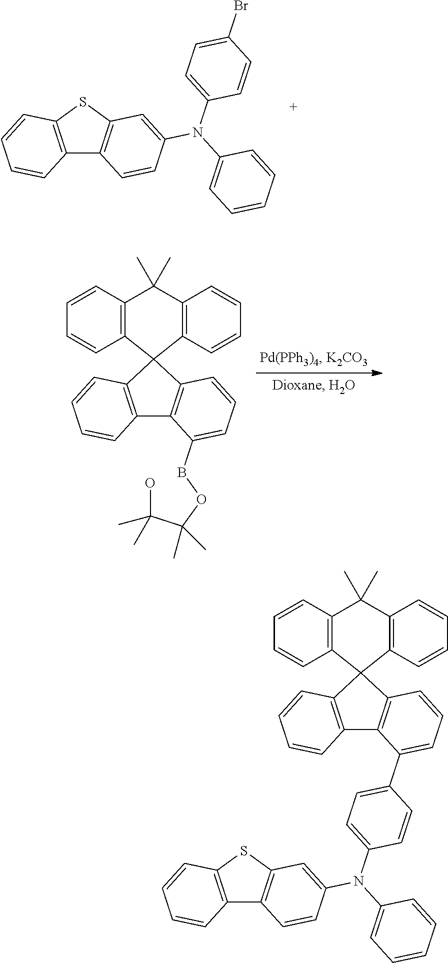

A target compound (5.3 g, yield 60%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 6 and N-(4-bromophenyl)-N-phenyldibenzo[b,d]thiophene-3-amine were used.

[LCMS]: 707

### [Synthesis Example 28] Synthesis of Compound 116

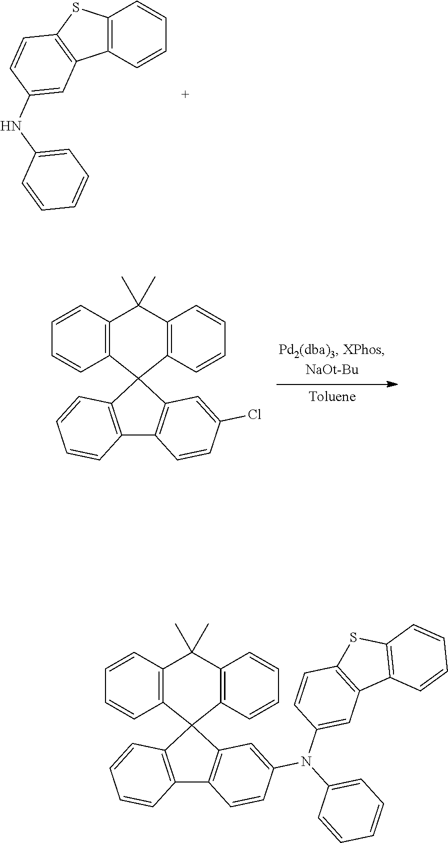 [SMILES: c1ccc(Nc2ccc3sc4ccccc4c3c2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccccc4)c4ccc5sc6ccccc6c5c4)cc32)c2ccccc21]

A target compound (3.3 g, yield 68%) was obtained in the same manner as in [Synthesis Example 1] except that 2′-chloro-10,10-dimethyl-10H-spiro[anthracene-9,9′-fluorene] of Preparation Example 1 and 2′-chloro-10,10-dimethyl-10H-spiro[anthracene-9,9′-fluorene] were used.

[LCMS]: 631

### [Synthesis Example 29] Synthesis of Compound 126

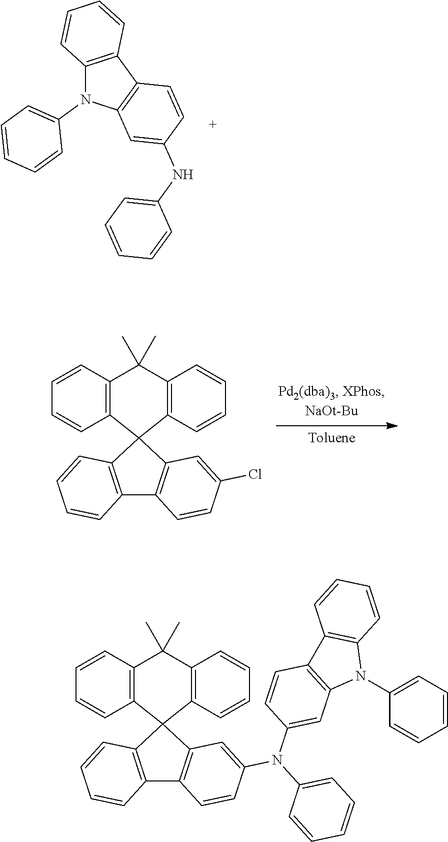 [SMILES: c1ccc(Nc2ccc3c4ccccc4n(-c4ccccc4)c3c2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccccc4)c4ccc5c6ccccc6n(-c6ccccc6)c5c4)cc32)c2ccccc21]

A target compound (5.3 g, yield 70%) was obtained in the carne manner as in [Synthesis Example 1] except that 2′-chloro-10,10-dimethyl-10H-spiro[anthracene-9,9′-fluorene] of Preparation Example 1 and N,9-diphenyl-9H-carbazole-2-amine were used.

[LCMS]: 690

### [Synthesis Example 30] Synthesis of Compound 129

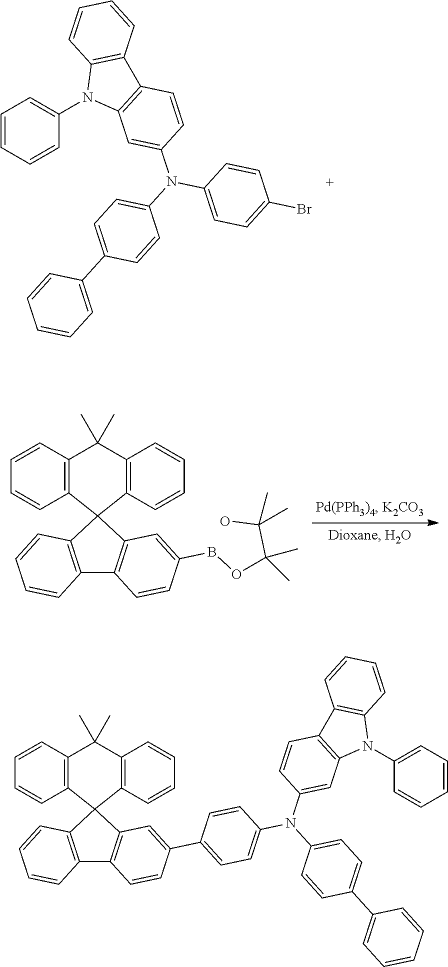

A target compound (2.7 g, yield 59%) was obtained in the came manner as in [Synthesis Example 3] except that 2-(10,10-dimethyl-10H-spiro[anthracene-9,9′-fluoren]-2′-yl)-4,4,5,5-tetramethyl-1,3,2-dioxaborolane of Preparation Example 4 and N-([1,1′-biphenyl]-4-yl)-N-(4-bromophenyl)-9-phenyl-9H-carbazole-2-amine were used.

[LCMS]: 843

### [Synthesis Example 31] Synthesis of Compound 136

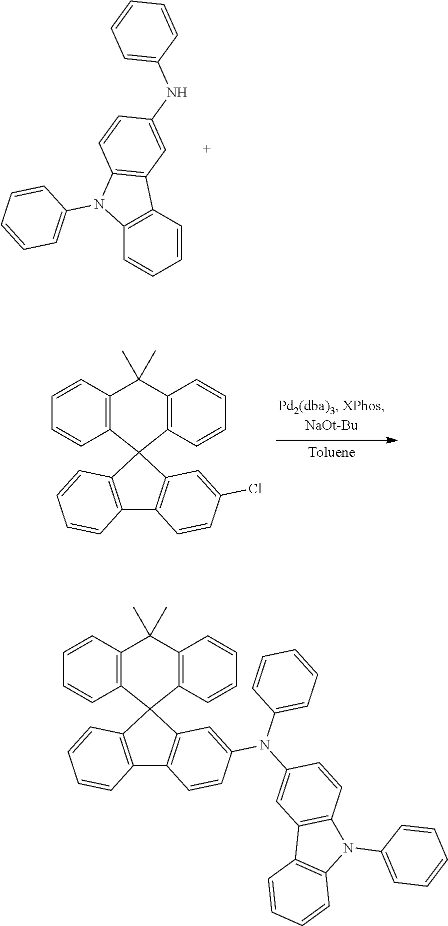 [SMILES: c1ccc(Nc2ccc3c(c2)c2ccccc2n3-c2ccccc2)cc1 ; CC1(C)c2ccccc2C2(c3ccccc3-c3ccc(Cl)cc32)c2ccccc21 ; CC1(C)c2ccccc2C2(c3ccccc3-c3ccc(N(c4ccccc4)c4ccc5c(c4)c4ccccc4n5-c4ccccc4)cc32)c2ccccc21]

A target compound (7.2 g, yield 54%) was obtained in the same manner as in [Synthesis Example 1] except that 2′-chloro-10,10-dimethyl-10H-spiro[anthracene-9,9′-fluorene] of Preparation Example 1 and N,9-diphenyl-9H-carbazole-3-amine were used.

[LCMS]: 690

### [Synthesis Example 32] Synthesis of Compound 143

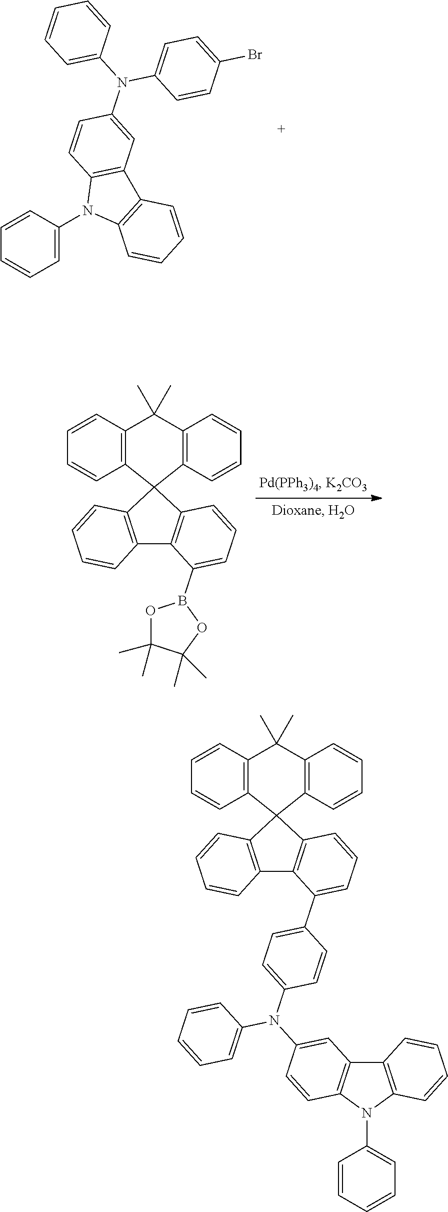 [SMILES: Brc1ccc(N(c2ccccc2)c2ccc3c(c2)c2ccccc2n3-c2ccccc2)cc1 ; CC1(C)c2ccccc2C2(c3ccccc3-c3c(B4OC(C)(C)C(C)(C)O4)cccc32)c2ccccc21 ; CC1(C)c2ccccc2C2(c3ccccc3-c3c(-c4ccc(N(c5ccccc5)c5ccc6c(c5)c5ccccc5n6-c5ccccc5)cc4)cccc32)c2ccccc21]

A target compound (4.3 g, yield 60%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 6 and N-(4-bromophenyl)-N,9-diphenyl-9H-carbazole-3-amine were used

[LCMS]: 766

### [Synthesis Example 33] Synthesis of Compound 149

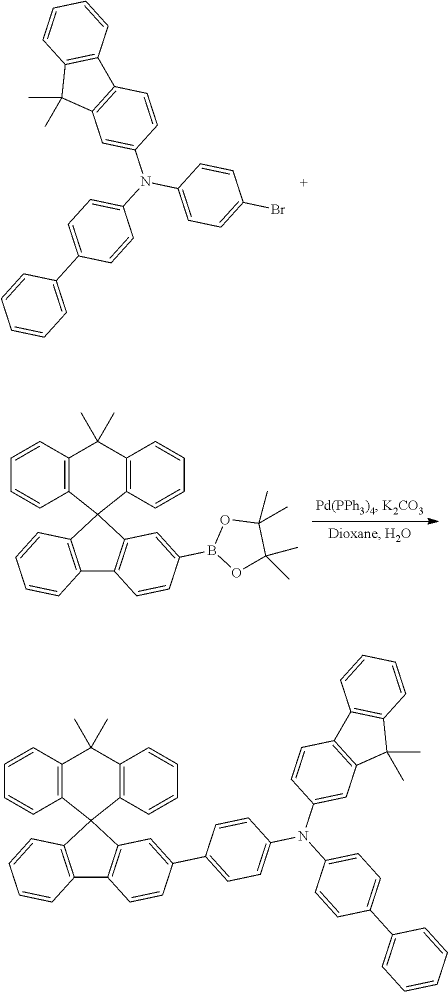 [SMILES: CC1(C)c2ccccc2-c2ccc(N(c3ccc(Br)cc3)c3ccc(-c4ccccc4)cc3)cc21 ; CC1(C)c2ccccc2C2(c3ccccc3-c3ccc(B4OC(C)(C)C(C)(C)O4)cc32)c2ccccc21 ; CC1(C)c2ccccc2-c2ccc(N(c3ccc(-c4ccccc4)cc3)c3ccc(-c4ccc5c(c4)C4(c6ccccc6-5)c5ccccc5C(C)(C)c5ccccc54)cc3)cc21]

A target compound (3.5 g, yield 65%) was obtained in the same manner as in [Synthesis Example 3] except that 2-(10,10-dimethyl-10H-spiro[anthracene-9,9′-fluoren]-2′-yl)-4,4,5,5-tetramethyl-1,3,2-dioxaborolane of Preparation Example 4 and N-([1,1′-biphenyl]-4-yl)-N-(4-bromophenyl)-9,9-dimethyl-9H-fluorene-2-amine were used.

[LCMS]: 794

### [Synthesis Example 34] Synthesis of Compound 154

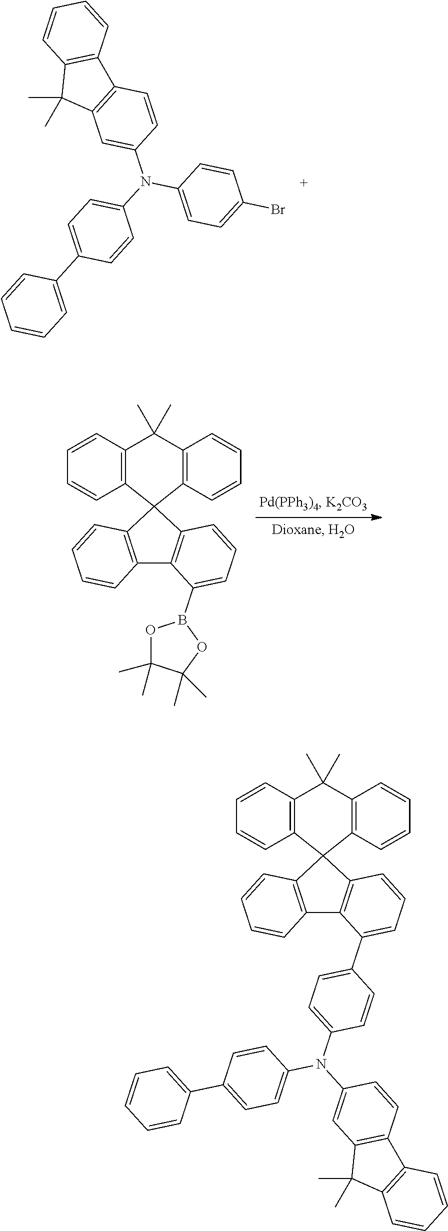 [SMILES: CC1(C)c2ccccc2-c2ccc(N(c3ccc(Br)cc3)c3ccc(-c4ccccc4)cc3)cc21 ; CC1(C)c2ccccc2C2(c3ccccc3-c3c(B4OC(C)(C)C(C)(C)O4)cccc32)c2ccccc21 ; CC1(C)c2ccccc2-c2ccc(N(c3ccc(-c4ccccc4)cc3)c3ccc(-c4cccc5c4-c4ccccc4C54c5ccccc5C(C)(C)c5ccccc54)cc3)cc21]

A target compound (6.9 g, yield 52%) was obtained in the same manner as in [Synthesis Example 3] except that 2-(10,10-dimethyl-10H-spiro[anthracene-9,9′-fluoren]-4′-yl)-4,4,5,5-tetramethyl-1,3,2-dioxaborolane of Preparation Example 6 and N-([1,1′-biphenyl]-4 yl)-N-(4-bromophenyl)-9,9-dimethyl-9H-fluorene-2-amine were used.

[LCMS]: 794

### [Examples 1 to 34] Manufacture of Organic Electroluminescent Device

After high purity sublimation purifying Compounds 2, 4, 6, 8, 12, 14, 16, 19, 22, 24, 26, 30, 34, 36, 42, 44, 51, 57, 62, 67, 72, 79, 92, 97, 107, 113, 116, 126, 129, 136, 143, 149 and 154 synthesized in the synthesis examples using commonly known methods, green organic electroluminescent devices were manufactured using the following procedure.

First, a glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning for 5 minutes using UV, the coated glass substrate was transferred to a vacuum deposition apparatus.

On the transparent ITO glass substrate (electrode) prepared as above, m-MTDATA (60 nm)/each compound of 2, 4, 6, 8, 12, 14, 16, 19, 22, 24, 26, 30, 34, 36, 42, 44, 51, 57, 62, 67, 72, 79, 92, 97, 107, 113, 116, 126, 129, 136, 143, 149 and 154 (80 nm)/DS-H522+5% DS-501 (300 nm)BCP (10 nm)/Alq₃ (30 nm)/LiF (1 nm)/Al (200 nm) were laminated in this order to manufacture an organic EL device.

DS-H522 and DS-501 used for manufacturing the device were products manufactured by Doosan Corporation Electro-Materials BG, and structures of m-MTDATA, TCTA, CBP, Ir(ppy)₃, and BCP are as follows.

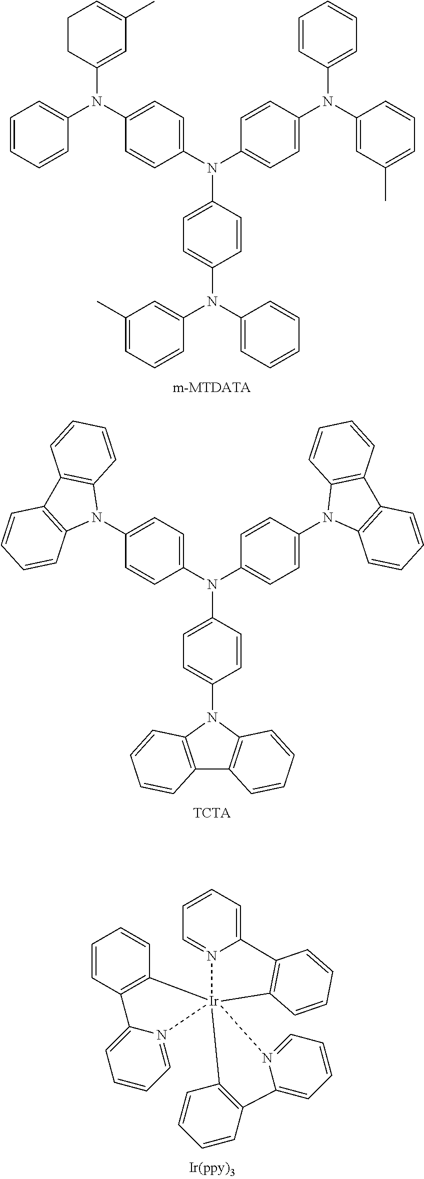

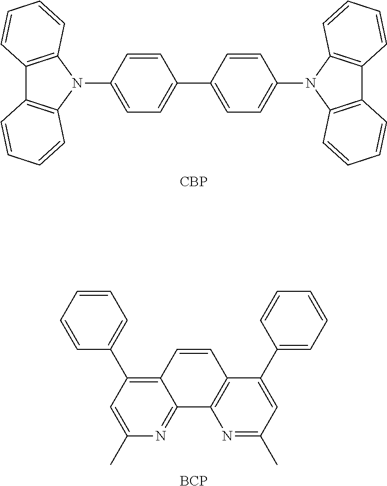

### [Comparative Example 1] Manufacture of Organic Electroluminescent Device

An organic EL device was manufactured in the same manner as in Example 1 except that NPB was used as the hole transport layer material instead of Compound 2 used as the hole transport layer material when forming the hole transport layer. A structure of the used NPB is as follows.

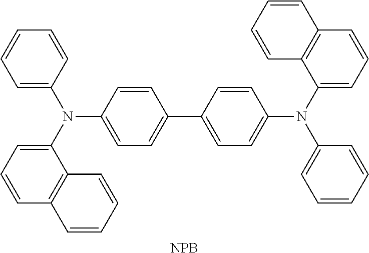

### Evaluation Example 1

For each of the green organic electroluminescent devices manufactured in Examples 1 to 34 and Comparative Example 1, driving voltage, current efficiency and light emission peak at current density of 10 mA/cm² were measured, and the results are shown in the following Table 1.

TABLE 1

| | | | Current |
|---|---|---|---|
| | Hole Transport | Driving | Efficiency |
| Sample | Layer | Voltage (V) | (cd/A) |
| Example 1 | Compound 2 | 4.3 | 24.3 |
| Example 2 | Compound 4 | 4.1 | 22.9 |
| Example 3 | Compound 6 | 4.1 | 23 |
| Example 4 | Compound 8 | 4.5 | 23.8 |
| Example 5 | Compound 12 | 5 | 21.9 |
| Example 6 | Compound 14 | 3.9 | 20.5 |
| Example 7 | Compound 16 | 4.3 | 21.5 |
| Example 8 | Compound 19 | 4.8 | 22.6 |
| Example 9 | Compound 22 | 4.2 | 23.7 |
| Example 10 | Compound 24 | 3.9 | 22 |
| Example 11 | Compound 26 | 4.1 | 21.9 |
| Example 12 | Compound 30 | 3.7 | 20.9 |
| Example 13 | Compound 34 | 3.9 | 22.4 |
| Example 14 | Compound 36 | 4.2 | 22.5 |
| Example 15 | Compound 42 | 4.5 | 23.9 |
| Example 16 | Compound 44 | 4.1 | 24.1 |
| Example 17 | Compound 51 | 4.4 | 21 |
| Example 18 | Compound 54 | 3.9 | 20.3 |
| Example 19 | Compound 57 | 4.3 | 19.4 |
| Example 20 | Compound 62 | 4.6 | 21.9 |
| Example 21 | Compound 67 | 4.1 | 22.7 |
| Example 22 | Compound 72 | 4 | 23 |
| Example 23 | Compound 79 | 4.8 | 22 |
| Example 24 | Compound 92 | 4.5 | 24 |
| Example 25 | Compound 97 | 3.9 | 21.2 |
| Example 26 | Compound 107 | 4.1 | 22.1 |
| Example 27 | Compound 113 | 4.2 | 23 |
| Example 28 | Compound 116 | 4.1 | 22.8 |
| Example 29 | Compound 126 | 4.5 | 21 |
| Example 30 | Compound 129 | 4.7 | 22.8 |
| Example 31 | Compound 136 | 3.8 | 24.1 |
| Example 32 | Compound 143 | 5.1 | 20.2 |
| Example 33 | Compound 149 | 5 | 19.8 |
| Example 34 | Compound 154 | 4.4 | 23.5 |
| Comparative | NPB | 5.2 | 18.2 |
| Example 1 | | | |

As shown in Table 1, it was seen that the organic electroluminescent devices using the compounds according to the present invention in a hole transport layer (organic electroluminescent devices each manufactured in Examples 1 to 34) exhibited superior performance in terms of current efficiency and driving voltage compared to when using existing NBP (Comparative Example 1).

### [National Research and Development Program Supporting This Invention]

[Unique Project Number] NRF-2016M3A7B4909246
[Name of Ministry] Ministry of Science and ICT
[Research Management Organization] National Research Foundation of Korea
[Name of Research Program] Development of Nano-Material Original Technology
[Title of Research Project] Development of Blue Phosphorescent Dopant for High Efficiency and Long Lifetime AMOLED
[Contribution] 1/1
[Leading Organization] Kangwon National University
[Research Period] 2017.03.01-2018.02.28

### INDUSTRIAL APPLICABILITY

The present invention relates to a novel organic compound capable of being used as a material for an organic electroluminescent device, and an organic electroluminescent device including the same.

1. A compound represented by the following Chemical Formula 1: 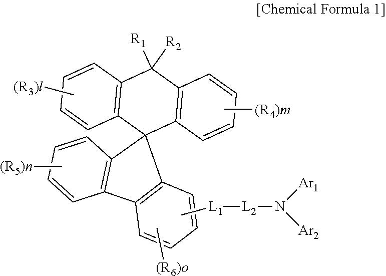
wherein, in Chemical Formula 1,
l, m and n are each independently an integer of 0 to 4;
o is an integer of 0 to 3;
R₁ and R₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group;
R₃ to R₆ are each independently selected from the group consisting of deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group, and when R₃ to R₆ are each present in plural numbers, these are the same as or different from each other;
the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of R₁ to R₆ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other;
L₁ and L₂ are each independently selected from the group consisting of a direct bond, a C₆-C₁₈ arylene group and a heteroarylene group having 5 to 18 nuclear atoms;
Ar₁ and Ar₂ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylamine group; and
the arylene group and the heteroarylene group of L₁ and L₂, and the alkyl group, the alkenyl group, the alkenyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphanyl group, the mono or diarylphosphinyl group and the arylsilyl group of Ar₁ and Ar₂ are each independently unsubstituted or substituted with one or more types of substituents selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphanyl group, a C₆˜C₆₀ mono or diarylphosphinyl group and a C₆˜C₆₀ arylsilyl group, and when substituted with a plurality of the substituents, these are the same as or different from each other.

2. The compound of claim 1, which is represented by any one of the following Chemical Formulae 2 to 4: 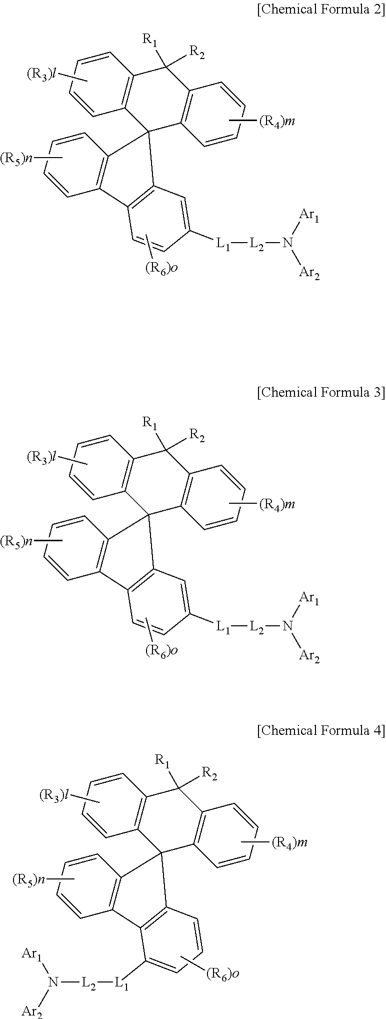
wherein, in Chemical Formulae 2 to 4,
R₁ to R₆, l, m, n, o, L₁, L₂, Ar₁ and Arₑ have the same definitions as in claim 1.

3. The compound of claim 1, wherein R₁ and R₂ are each independently selected from the group consisting of a C₁˜C₄₀ alkyl group, a C₆˜C₆₀ aryl group and a heteroaryl group having 5 to 60 nuclear atoms; and
the alkyl group, the aryl group and the heteroaryl group of R₁ and R₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

4. The compound of claim 1, wherein L₁ and L₂ are each independently a direct bond, or a linker selected from the group consisting of the following Chemical Formulae A-1 to A-4: 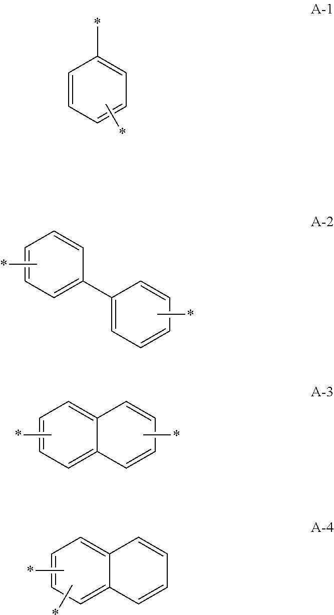
in Chemical Formulae A-1 to A-4,
* means a part where a bond is formed.

5. The compound of claim 1, wherein Ar₁ and Ar₂ are selected from the group consisting of a C₁˜C₄₀ alkyl group, a C₆˜C₆₀ aryl group and a heteroaryl group having 5 to 60 nuclear atoms; and
the alkyl group, the aryl group and the heteroaryl group of Ar₁ and Ar₂ are each independently unsubstituted or substituted with one or more types of substituents selected from the group consisting of a C₁˜C₄₀ alkyl group, a C₆˜C₆₀ aryl group and a heteroaryl group having 5 to 60 nuclear atoms, and when substituted with a plurality of the substituents, these are the same as or different from each other.

6. The compound of claim 1, wherein at least one of Ar₁ and Ar₂ is a substituent represented by the following Chemical Formula 5 or 6: 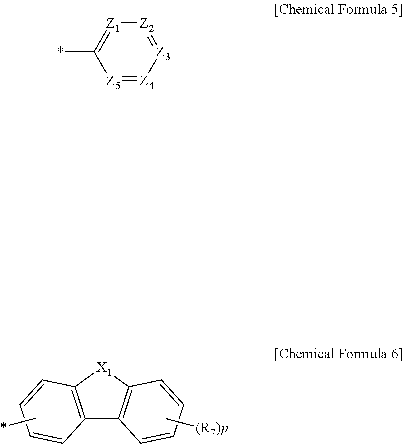
in Chemical Formulae 5 and 6,
* means a part where a bond is formed;
p is an integer of 0 to 4;
Z₁ to Z₅ are each independently N or C(R₈);
X₁ is O, S, N(R₉) or C(R₁₀)(R₁₁);
R₇ is selected from the group consisting of deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₃˜C₄₀ cycloalkyl group, a heterocycloalkyl group having 3 to 40 nuclear atoms, a C₆˜C₆₀ arylamine group, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, or bonds to adjacent groups to form a fused ring, and when R₇ is present in plural numbers, these are the same as or different from each other;
R₈ to R₁₁ are each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₃˜C₄₀ cycloalkyl group, a heterocycloalkyl group having 3 to 40 nuclear atoms, a C₆˜C₆₀ arylamine group, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, or bond to adjacent groups to form a fused ring, and when R₈ is present in plural numbers, these are the same as or different from each other; and
the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the mono or diarylphosphinyl group and the arylsilyl group of R₇ to R₁₁ are each independently unsubstituted or substituted with one or more types of substituents selected from the group consisting of deuterium, halogen, a cyano group, a nitro group, a C₁˜C₄₀ alkyl group, a C₂-C₄₀ alkenyl group, a C₂˜C₄₀ alkynyl group, a C₆˜C₆₀ aryl group, a heteroaryl group having 5 to 60 nuclear atoms, a C₆˜C₆₀ aryloxy group, a C₁˜C₄₀ alkyloxy group, a C₆˜C₆₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylsilyl group, and when substituted with a plurality of the substituents, these are the same as or different from each other.

7. The compound of claim 6, wherein R₉ to R₁₁ are each independently selected from the group consisting of a C₁˜C₄₀ alkyl group, a C₆˜C₆₀ aryl group and a heteroaryl group having 5 to 60 nuclear atoms.

8. The compound of claim 1, which is selected from the group consisting of the following compounds:

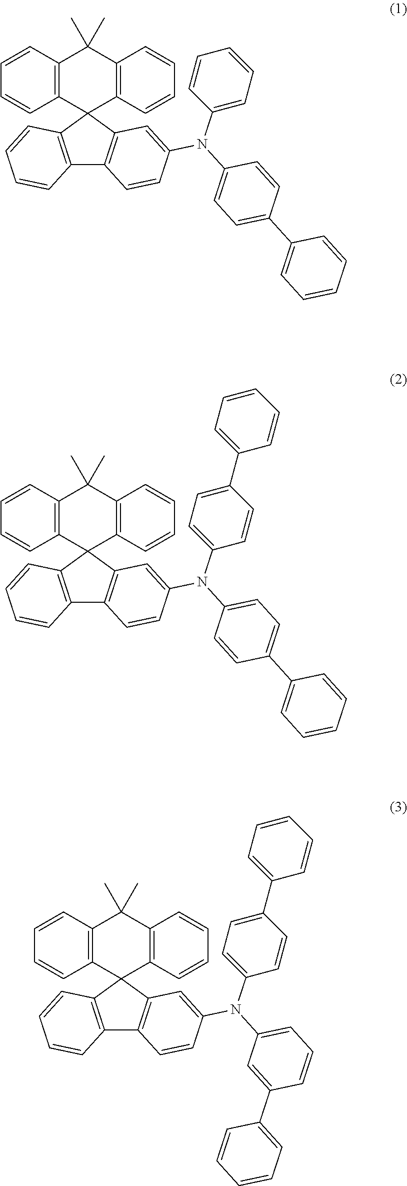 [SMILES: CC1(C)c2ccccc2C2(c3ccccc3-c3ccc(N(c4ccccc4)c4ccc(-c5ccccc5)cc4)cc32)c2ccccc21 ; CC1(C)c2ccccc2C2(c3ccccc3-c3ccc(N(c4ccc(-c5ccccc5)cc4)c4ccc(-c5ccccc5)cc4)cc32)c2ccccc21 ; CC1(C)c2ccccc2C2(c3ccccc3-c3ccc(N(c4ccc(-c5ccccc5)cc4)c4cccc(-c5ccccc5)c4)cc32)c2ccccc21]

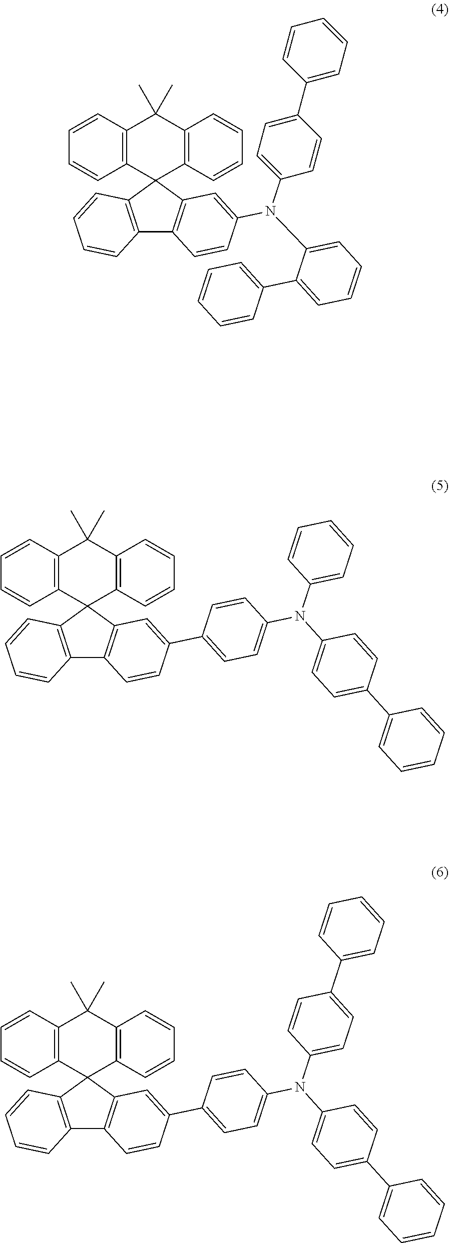 [SMILES: CC1(C)c2ccccc2C2(c3ccccc3-c3ccc(N(c4ccc(-c5ccccc5)cc4)c4ccccc4-c4ccccc4)cc32)c2ccccc21 ; CC1(C)c2ccccc2C2(c3ccccc3-c3ccc(-c4ccc(N(c5ccccc5)c5ccc(-c6ccccc6)cc5)cc4)cc32)c2ccccc21 ; CC1(C)c2ccccc2C2(c3ccccc3-c3ccc(-c4ccc(N(c5ccc(-c6ccccc6)cc5)c5ccc(-c6ccccc6)cc5)cc4)cc32)c2ccccc21]

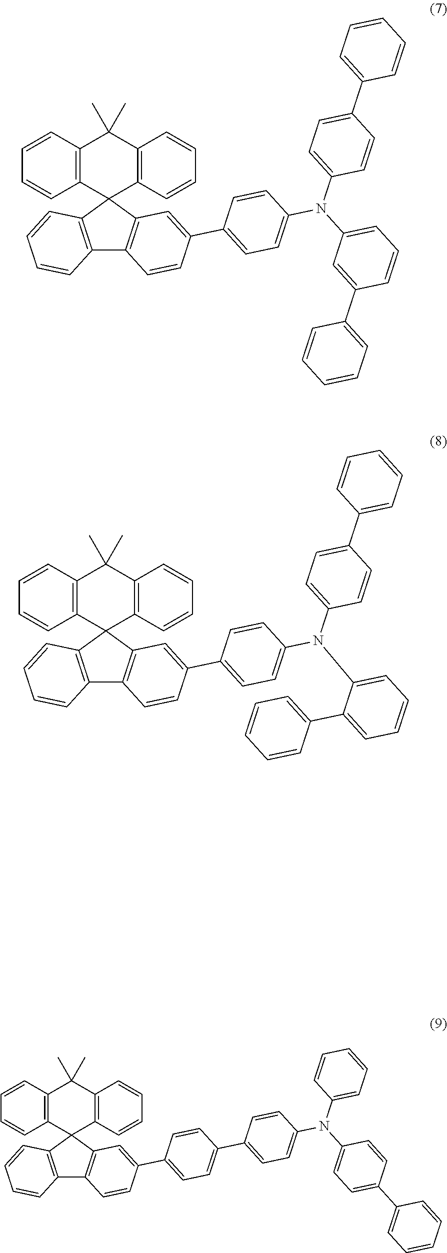 [SMILES: CC1(C)c2ccccc2C2(c3ccccc3-c3ccc(-c4ccc(N(c5ccc(-c6ccccc6)cc5)c5cccc(-c6ccccc6)c5)cc4)cc32)c2ccccc21 ; CC1(C)c2ccccc2C2(c3ccccc3-c3ccc(-c4ccc(N(c5ccc(-c6ccccc6)cc5)c5ccccc5-c5ccccc5)cc4)cc32)c2ccccc21 ; CC1(C)c2ccccc2C2(c3ccccc3-c3ccc(-c4ccc(-c5ccc(N(c6ccccc6)c6ccc(-c7ccccc7)cc6)cc5)cc4)cc32)c2ccccc21]

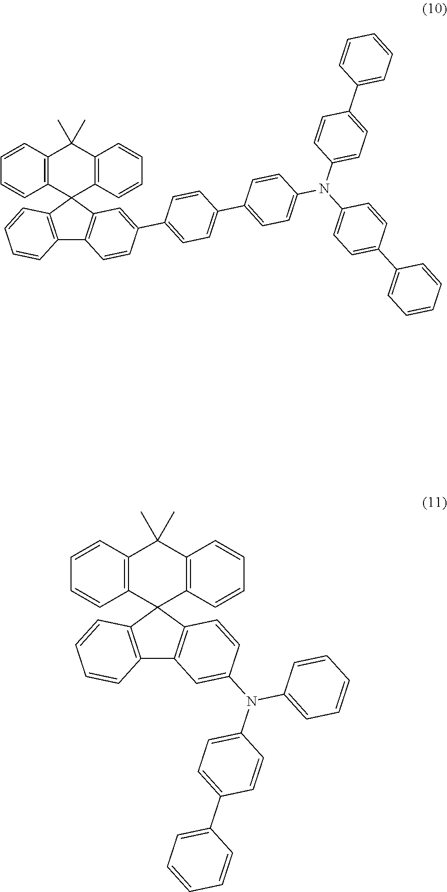

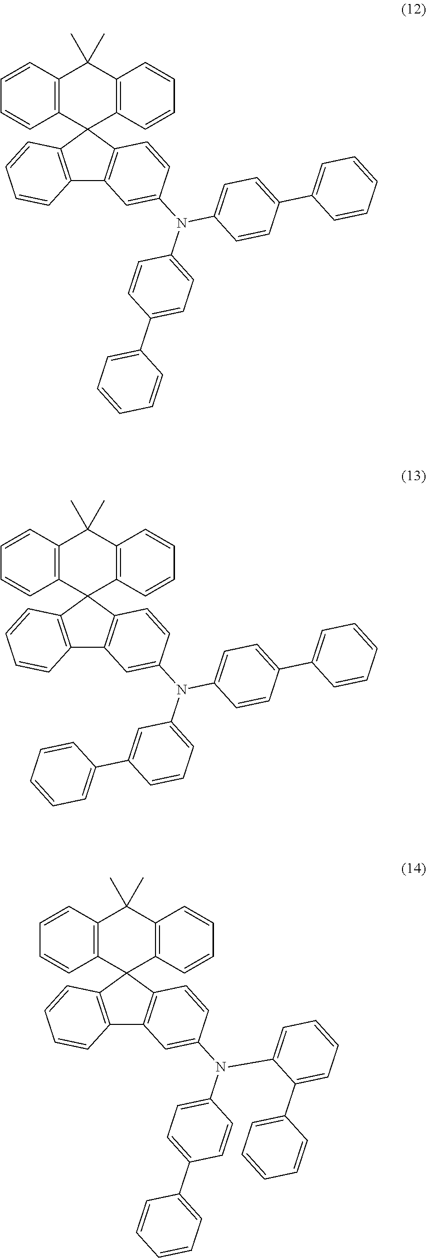 [SMILES: CC1(C)c2ccccc2C2(c3ccccc3-c3cc(N(c4ccc(-c5ccccc5)cc4)c4ccc(-c5ccccc5)cc4)ccc32)c2ccccc21 ; CC1(C)c2ccccc2C2(c3ccccc3-c3cc(N(c4ccc(-c5ccccc5)cc4)c4cccc(-c5ccccc5)c4)ccc32)c2ccccc21 ; CC1(C)c2ccccc2C2(c3ccccc3-c3cc(N(c4ccc(-c5ccccc5)cc4)c4ccccc4-c4ccccc4)ccc32)c2ccccc21]

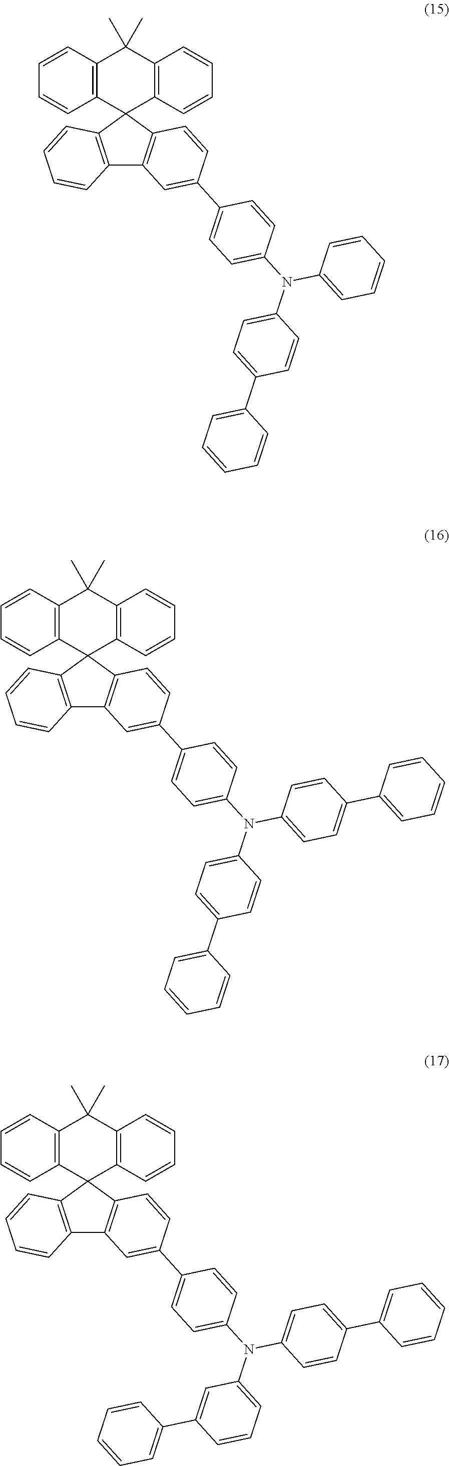 [SMILES: CC1(C)c2ccccc2C2(c3ccccc3-c3cc(-c4ccc(N(c5ccccc5)c5ccc(-c6ccccc6)cc5)cc4)ccc32)c2ccccc21 ; CC1(C)c2ccccc2C2(c3ccccc3-c3cc(-c4ccc(N(c5ccc(-c6ccccc6)cc5)c5ccc(-c6ccccc6)cc5)cc4)ccc32)c2ccccc21 ; CC1(C)c2ccccc2C2(c3ccccc3-c3cc(-c4ccc(N(c5ccc(-c6ccccc6)cc5)c5cccc(-c6ccccc6)c5)cc4)ccc32)c2ccccc21]

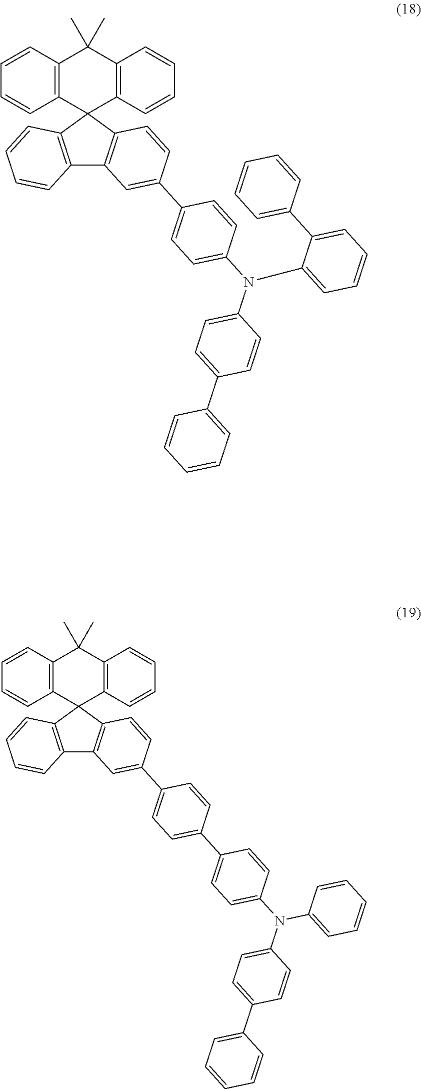 [SMILES: CC1(C)c2ccccc2C2(c3ccccc3-c3cc(-c4ccc(N(c5ccc(-c6ccccc6)cc5)c5ccccc5-c5ccccc5)cc4)ccc32)c2ccccc21 ; CC1(C)c2ccccc2C2(c3ccccc3-c3cc(-c4ccc(-c5ccc(N(c6ccccc6)c6ccc(-c7ccccc7)cc6)cc5)cc4)ccc32)c2ccccc21]

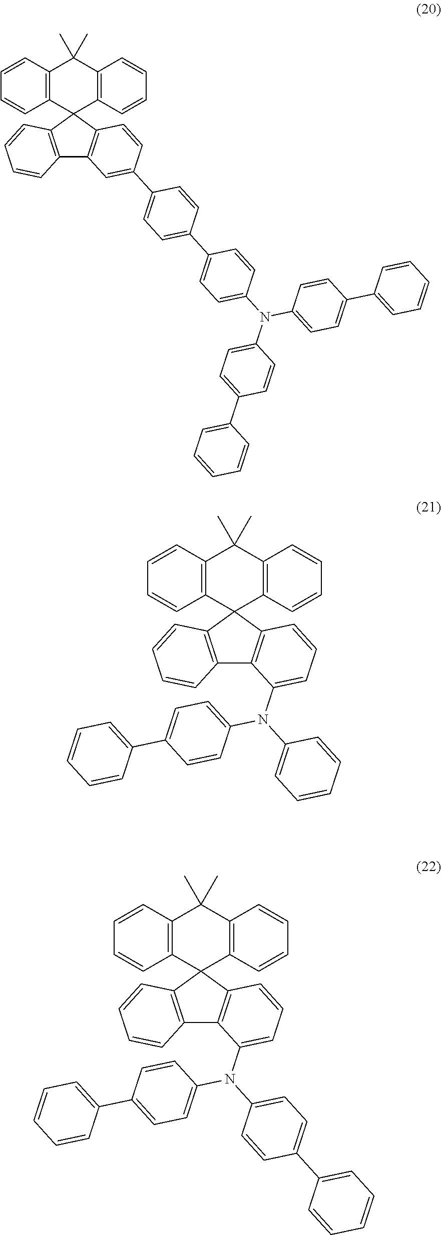

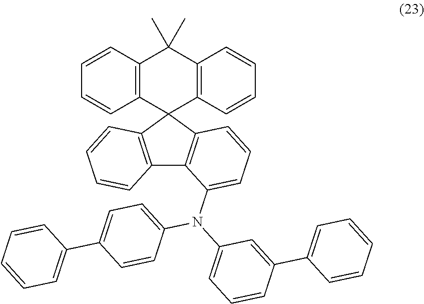 [SMILES: CC1(C)c2ccccc2C2(c3ccccc3-c3c(N(c4ccc(-c5ccccc5)cc4)c4cccc(-c5ccccc5)c4)cccc32)c2ccccc21]

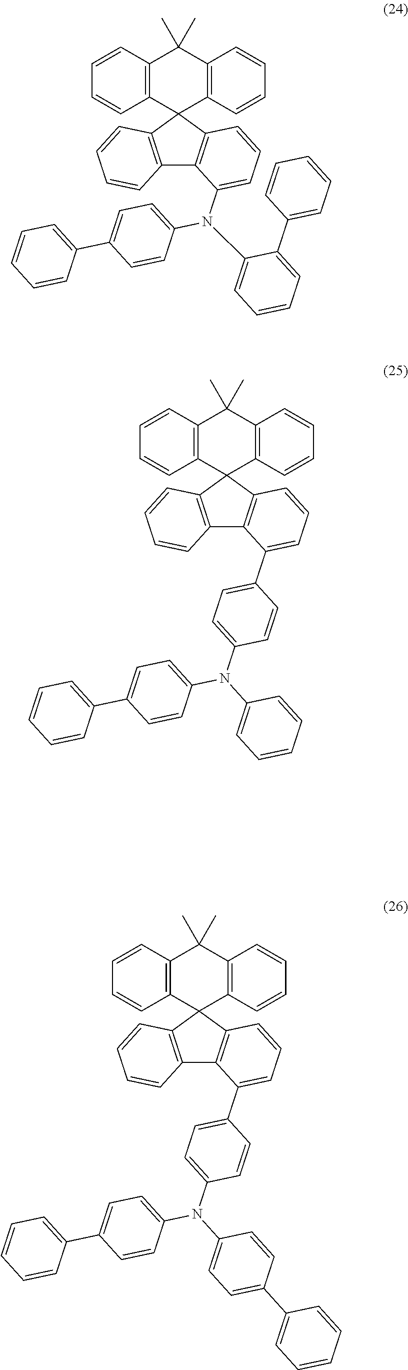 [SMILES: CC1(C)c2ccccc2C2(c3ccccc3-c3c(N(c4ccc(-c5ccccc5)cc4)c4ccccc4-c4ccccc4)cccc32)c2ccccc21 ; CC1(C)c2ccccc2C2(c3ccccc3-c3c(-c4ccc(N(c5ccccc5)c5ccc(-c6ccccc6)cc5)cc4)cccc32)c2ccccc21 ; CC1(C)c2ccccc2C2(c3ccccc3-c3c(-c4ccc(N(c5ccc(-c6ccccc6)cc5)c5ccc(-c6ccccc6)cc5)cc4)cccc32)c2ccccc21]

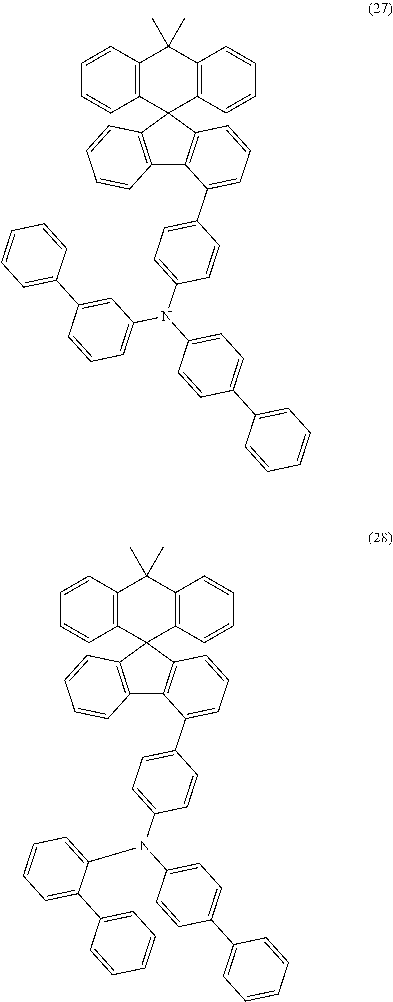 [SMILES: CC1(C)c2ccccc2C2(c3ccccc3-c3c(-c4ccc(N(c5ccc(-c6ccccc6)cc5)c5cccc(-c6ccccc6)c5)cc4)cccc32)c2ccccc21 ; CC1(C)c2ccccc2C2(c3ccccc3-c3c(-c4ccc(N(c5ccc(-c6ccccc6)cc5)c5ccccc5-c5ccccc5)cc4)cccc32)c2ccccc21]

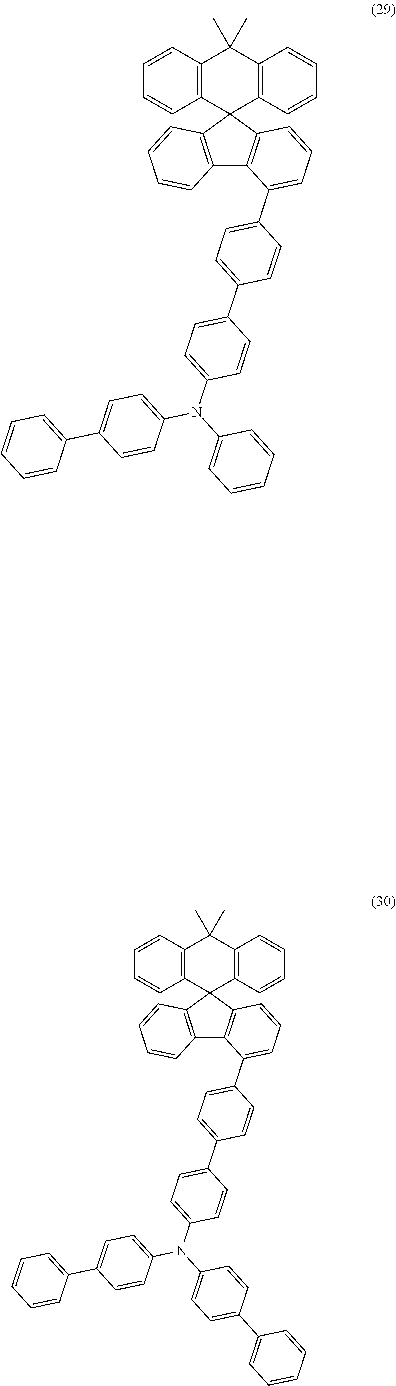 [SMILES: CC1(C)c2ccccc2C2(c3ccccc3-c3c(-c4ccc(-c5ccc(N(c6ccccc6)c6ccc(-c7ccccc7)cc6)cc5)cc4)cccc32)c2ccccc21 ; CC1(C)c2ccccc2C2(c3ccccc3-c3c(-c4ccc(-c5ccc(N(c6ccc(-c7ccccc7)cc6)c6ccc(-c7ccccc7)cc6)cc5)cc4)cccc32)c2ccccc21]

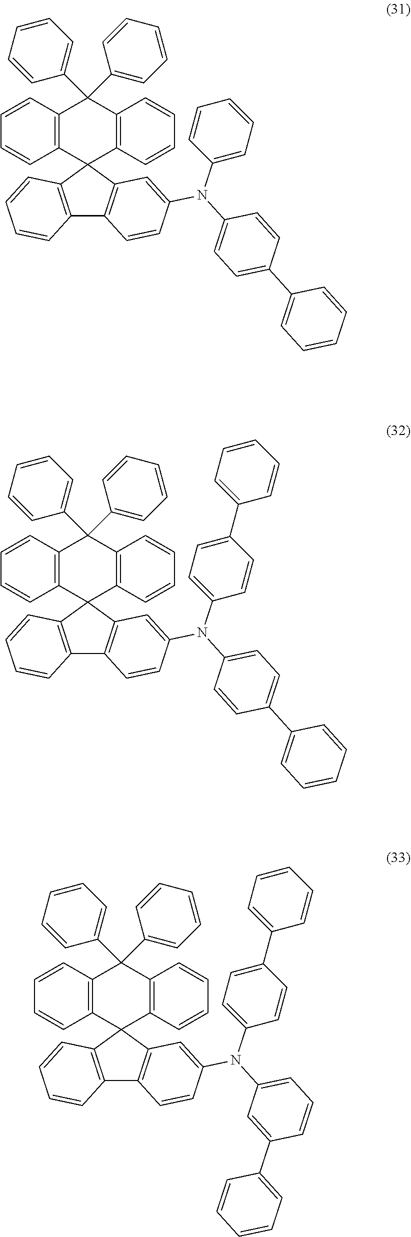 [SMILES: c1ccc(-c2ccc(N(c3ccccc3)c3ccc4c(c3)C3(c5ccccc5-4)c4ccccc4C(c4ccccc4)(c4ccccc4)c4ccccc43)cc2)cc1 ; c1ccc(-c2ccc(N(c3ccc(-c4ccccc4)cc3)c3ccc4c(c3)C3(c5ccccc5-4)c4ccccc4C(c4ccccc4)(c4ccccc4)c4ccccc43)cc2)cc1 ; c1ccc(-c2ccc(N(c3cccc(-c4ccccc4)c3)c3ccc4c(c3)C3(c5ccccc5-4)c4ccccc4C(c4ccccc4)(c4ccccc4)c4ccccc43)cc2)cc1]

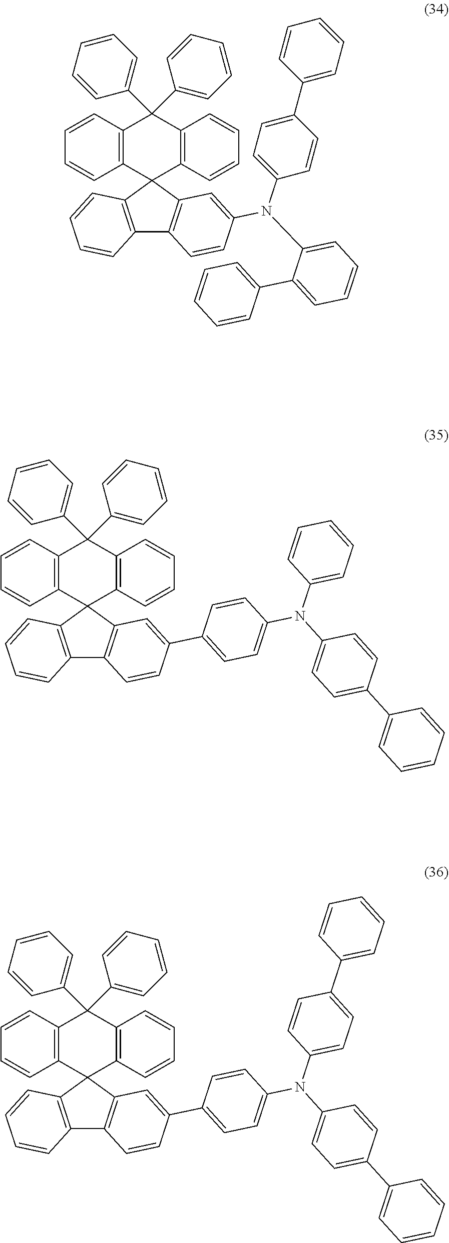 [SMILES: c1ccc(-c2ccc(N(c3ccc4c(c3)C3(c5ccccc5-4)c4ccccc4C(c4ccccc4)(c4ccccc4)c4ccccc43)c3ccccc3-c3ccccc3)cc2)cc1 ; c1ccc(-c2ccc(N(c3ccccc3)c3ccc(-c4ccc5c(c4)C4(c6ccccc6-5)c5ccccc5C(c5ccccc5)(c5ccccc5)c5ccccc54)cc3)cc2)cc1 ; c1ccc(-c2ccc(N(c3ccc(-c4ccccc4)cc3)c3ccc(-c4ccc5c(c4)C4(c6ccccc6-5)c5ccccc5C(c5ccccc5)(c5ccccc5)c5ccccc54)cc3)cc2)cc1]

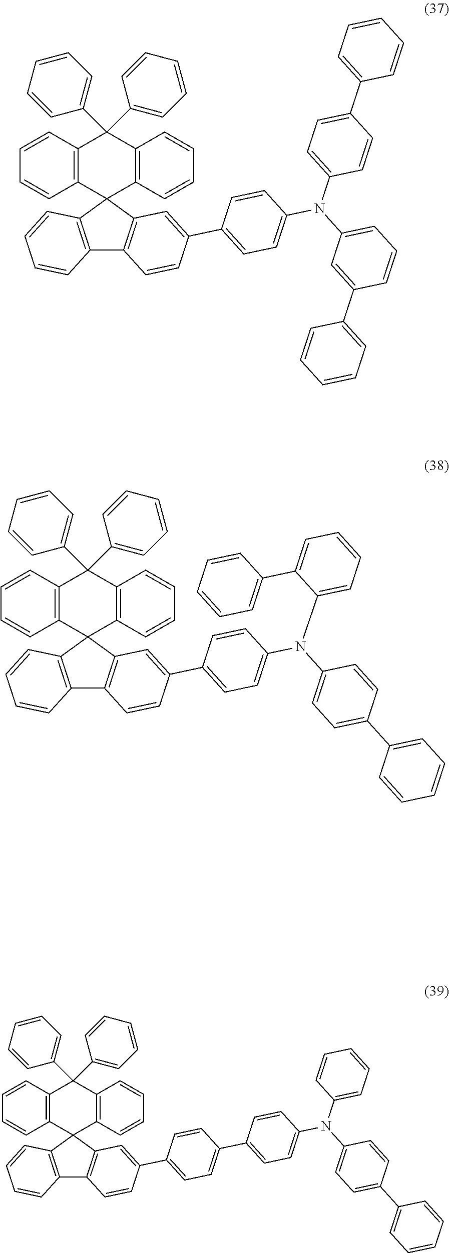 [SMILES: c1ccc(-c2ccc(N(c3ccc(-c4ccc5c(c4)C4(c6ccccc6-5)c5ccccc5C(c5ccccc5)(c5ccccc5)c5ccccc54)cc3)c3cccc(-c4ccccc4)c3)cc2)cc1 ; c1ccc(-c2ccc(N(c3ccc(-c4ccc5c(c4)C4(c6ccccc6-5)c5ccccc5C(c5ccccc5)(c5ccccc5)c5ccccc54)cc3)c3ccccc3-c3ccccc3)cc2)cc1 ; c1ccc(-c2ccc(N(c3ccccc3)c3ccc(-c4ccc(-c5ccc6c(c5)C5(c7ccccc7-6)c6ccccc6C(c6ccccc6)(c6ccccc6)c6ccccc65)cc4)cc3)cc2)cc1]

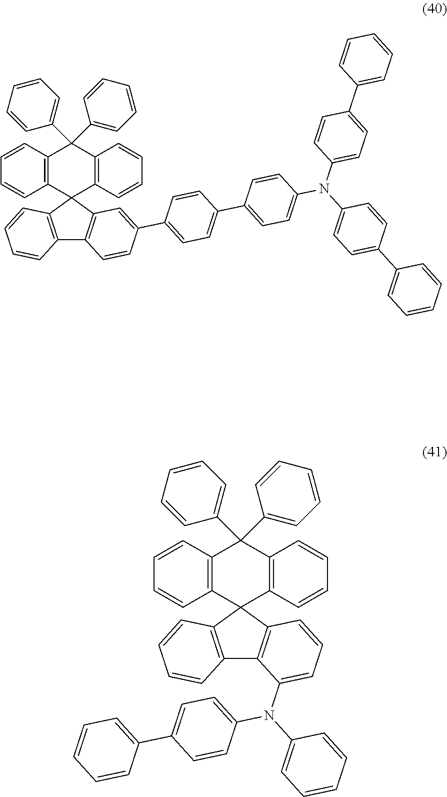

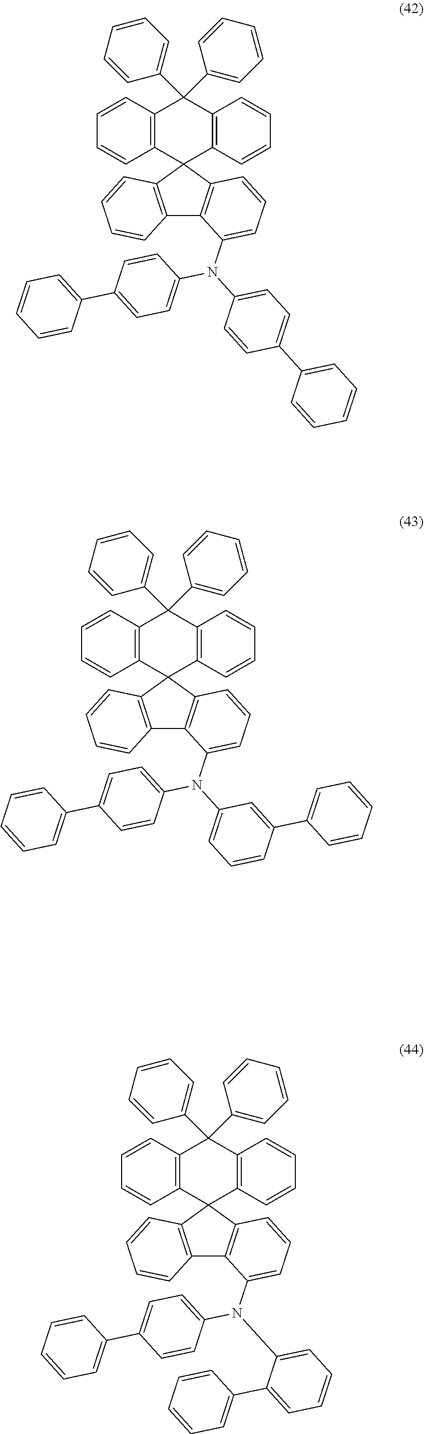 [SMILES: c1ccc(-c2ccc(N(c3ccc(-c4ccccc4)cc3)c3cccc4c3-c3ccccc3C43c4ccccc4C(c4ccccc4)(c4ccccc4)c4ccccc43)cc2)cc1 ; c1ccc(-c2ccc(N(c3cccc(-c4ccccc4)c3)c3cccc4c3-c3ccccc3C43c4ccccc4C(c4ccccc4)(c4ccccc4)c4ccccc43)cc2)cc1 ; c1ccc(-c2ccc(N(c3ccccc3-c3ccccc3)c3cccc4c3-c3ccccc3C43c4ccccc4C(c4ccccc4)(c4ccccc4)c4ccccc43)cc2)cc1]

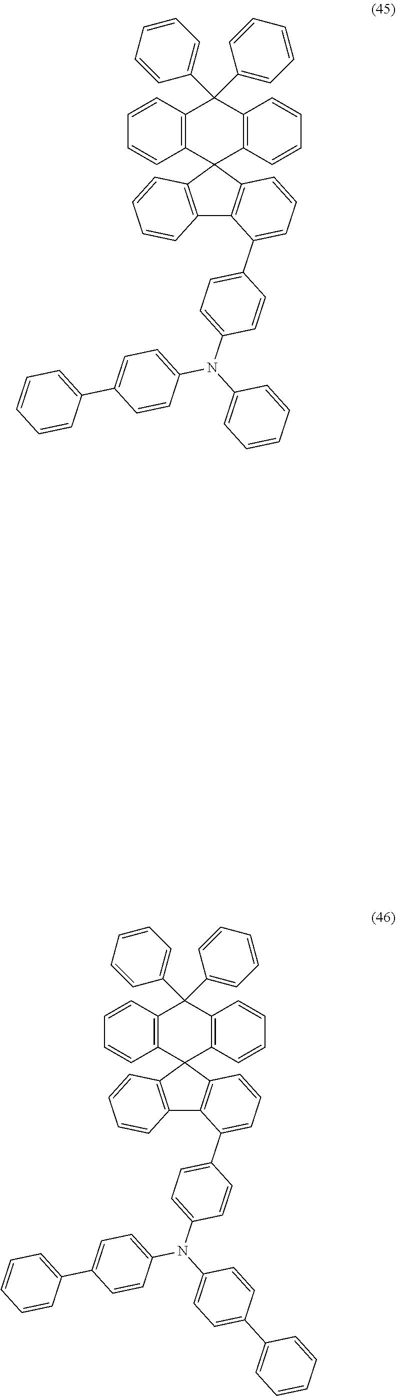 [SMILES: c1ccc(-c2ccc(N(c3ccccc3)c3ccc(-c4cccc5c4-c4ccccc4C54c5ccccc5C(c5ccccc5)(c5ccccc5)c5ccccc54)cc3)cc2)cc1 ; c1ccc(-c2ccc(N(c3ccc(-c4ccccc4)cc3)c3ccc(-c4cccc5c4-c4ccccc4C54c5ccccc5C(c5ccccc5)(c5ccccc5)c5ccccc54)cc3)cc2)cc1]

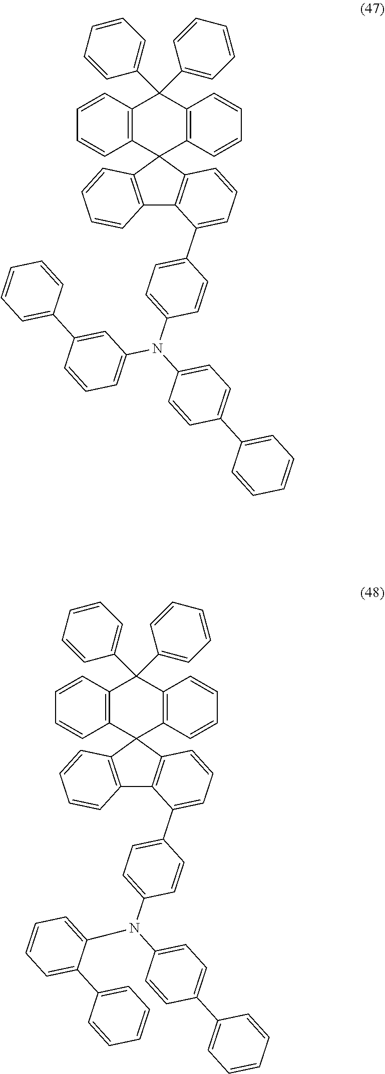 [SMILES: c1ccc(-c2ccc(N(c3ccc(-c4cccc5c4-c4ccccc4C54c5ccccc5C(c5ccccc5)(c5ccccc5)c5ccccc54)cc3)c3cccc(-c4ccccc4)c3)cc2)cc1 ; c1ccc(-c2ccc(N(c3ccc(-c4cccc5c4-c4ccccc4C54c5ccccc5C(c5ccccc5)(c5ccccc5)c5ccccc54)cc3)c3ccccc3-c3ccccc3)cc2)cc1]

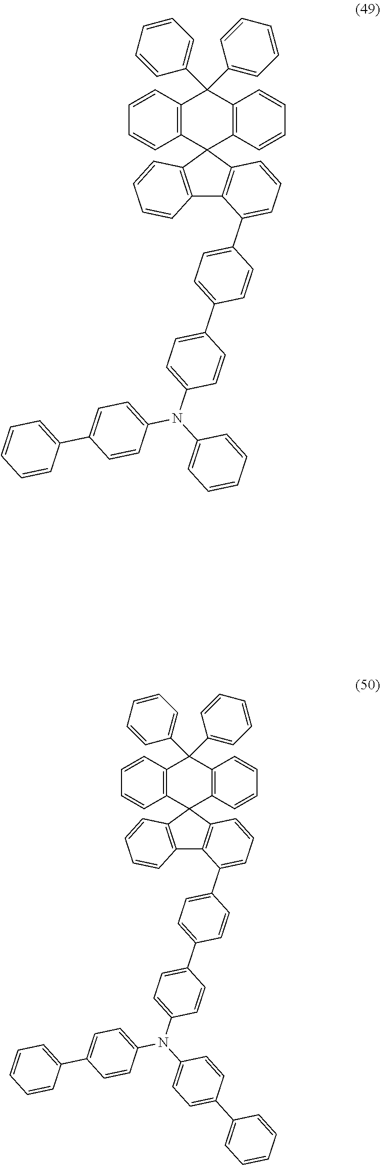 [SMILES: c1ccc(-c2ccc(N(c3ccccc3)c3ccc(-c4ccc(-c5cccc6c5-c5ccccc5C65c6ccccc6C(c6ccccc6)(c6ccccc6)c6ccccc65)cc4)cc3)cc2)cc1 ; c1ccc(-c2ccc(N(c3ccc(-c4ccccc4)cc3)c3ccc(-c4ccc(-c5cccc6c5-c5ccccc5C65c6ccccc6C(c6ccccc6)(c6ccccc6)c6ccccc65)cc4)cc3)cc2)cc1]

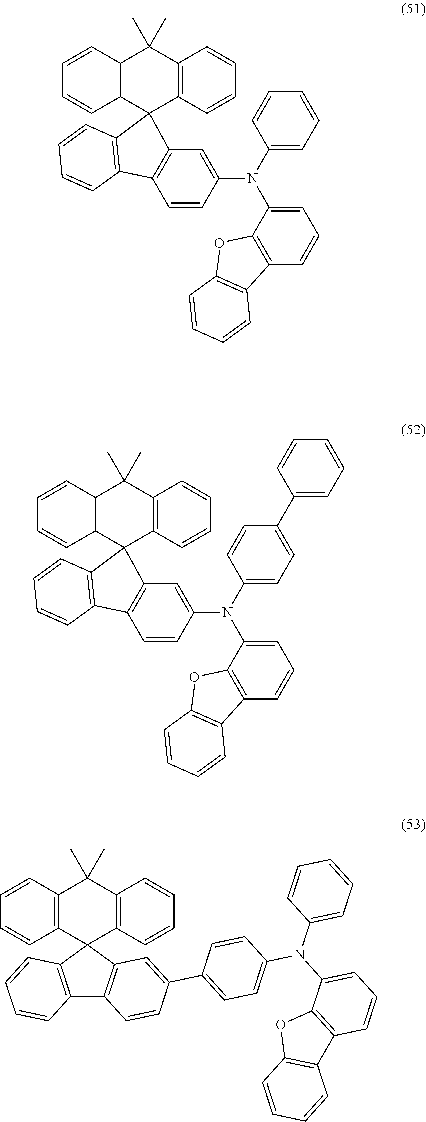 [SMILES: CC1(C)c2ccccc2C2(c3ccccc3-c3ccc(N(c4ccccc4)c4cccc5c4oc4ccccc45)cc32)C2C=CC=CC21 ; CC1(C)c2ccccc2C2(c3ccccc3-c3ccc(N(c4ccc(-c5ccccc5)cc4)c4cccc5c4oc4ccccc45)cc32)C2C=CC=CC21 ; CC1(C)c2ccccc2C2(c3ccccc3-c3ccc(-c4ccc(N(c5ccccc5)c5cccc6c5oc5ccccc56)cc4)cc32)c2ccccc21]

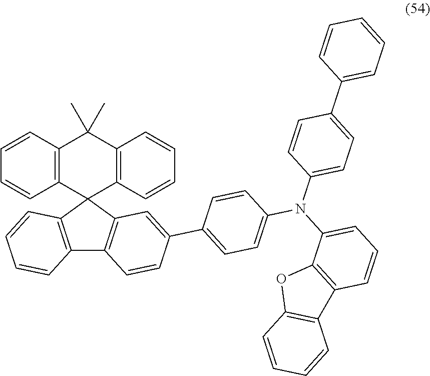 [SMILES: CC1(C)c2ccccc2C2(c3ccccc3-c3ccc(-c4ccc(N(c5ccc(-c6ccccc6)cc5)c5cccc6c5oc5ccccc56)cc4)cc32)c2ccccc21]

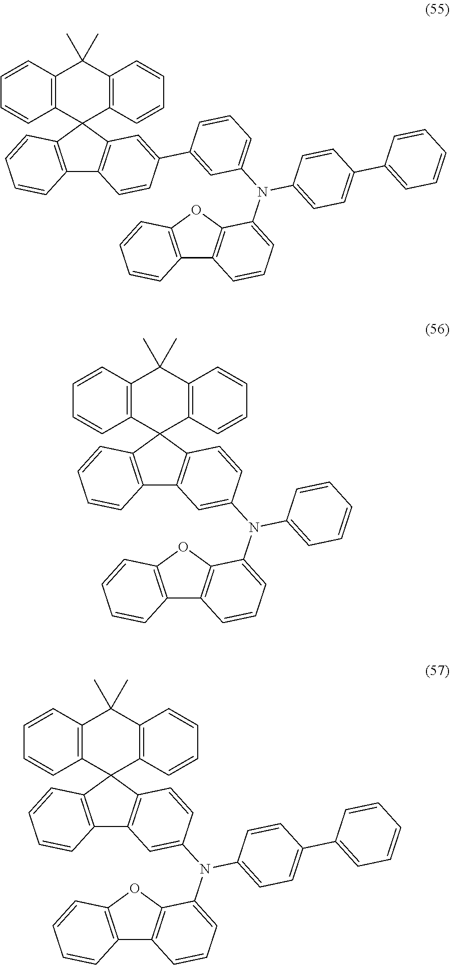 [SMILES: CC1(C)c2ccccc2C2(c3ccccc3-c3ccc(-c4cccc(N(c5ccc(-c6ccccc6)cc5)c5cccc6c5oc5ccccc56)c4)cc32)c2ccccc21 ; CC1(C)c2ccccc2C2(c3ccccc3-c3cc(N(c4ccccc4)c4cccc5c4oc4ccccc45)ccc32)c2ccccc21 ; CC1(C)c2ccccc2C2(c3ccccc3-c3cc(N(c4ccc(-c5ccccc5)cc4)c4cccc5c4oc4ccccc45)ccc32)c2ccccc21]

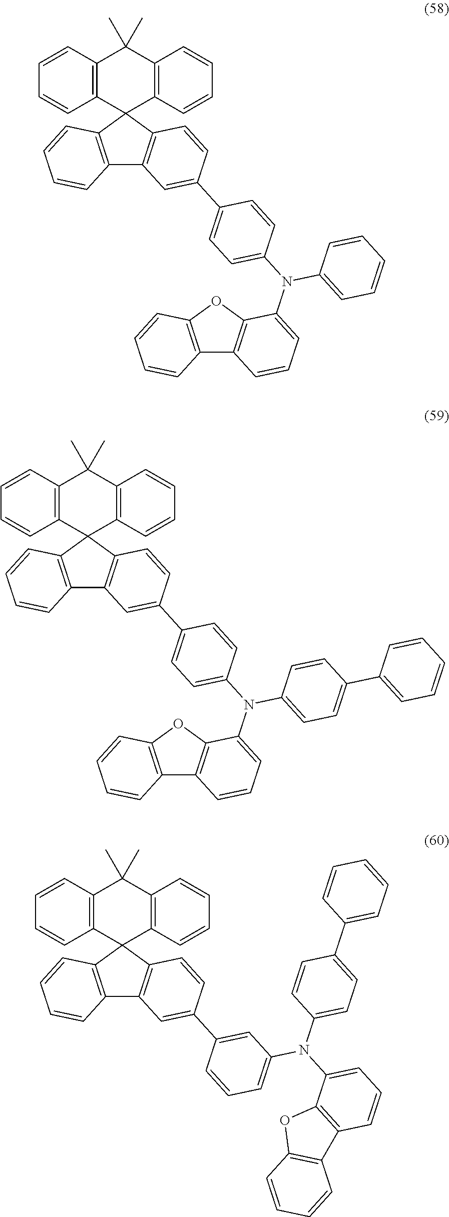 [SMILES: CC1(C)c2ccccc2C2(c3ccccc3-c3cc(-c4ccc(N(c5ccccc5)c5cccc6c5oc5ccccc56)cc4)ccc32)c2ccccc21 ; CC1(C)c2ccccc2C2(c3ccccc3-c3cc(-c4ccc(N(c5ccc(-c6ccccc6)cc5)c5cccc6c5oc5ccccc56)cc4)ccc32)c2ccccc21 ; CC1(C)c2ccccc2C2(c3ccccc3-c3cc(-c4cccc(N(c5ccc(-c6ccccc6)cc5)c5cccc6c5oc5ccccc56)c4)ccc32)c2ccccc21]

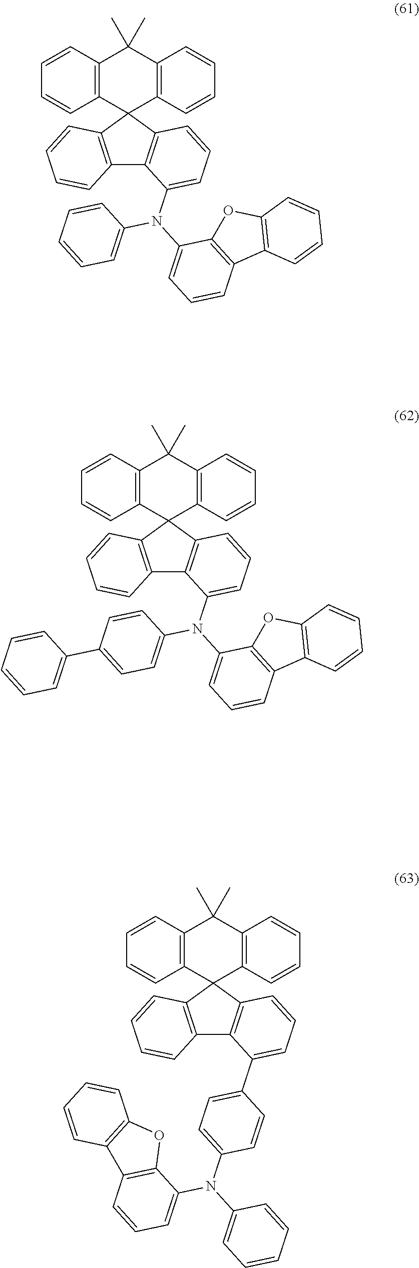 [SMILES: CC1(C)c2ccccc2C2(c3ccccc3-c3c(N(c4ccccc4)c4cccc5c4oc4ccccc45)cccc32)c2ccccc21 ; CC1(C)c2ccccc2C2(c3ccccc3-c3c(N(c4ccc(-c5ccccc5)cc4)c4cccc5c4oc4ccccc45)cccc32)c2ccccc21 ; CC1(C)c2ccccc2C2(c3ccccc3-c3c(-c4ccc(N(c5ccccc5)c5cccc6c5oc5ccccc56)cc4)cccc32)c2ccccc21]

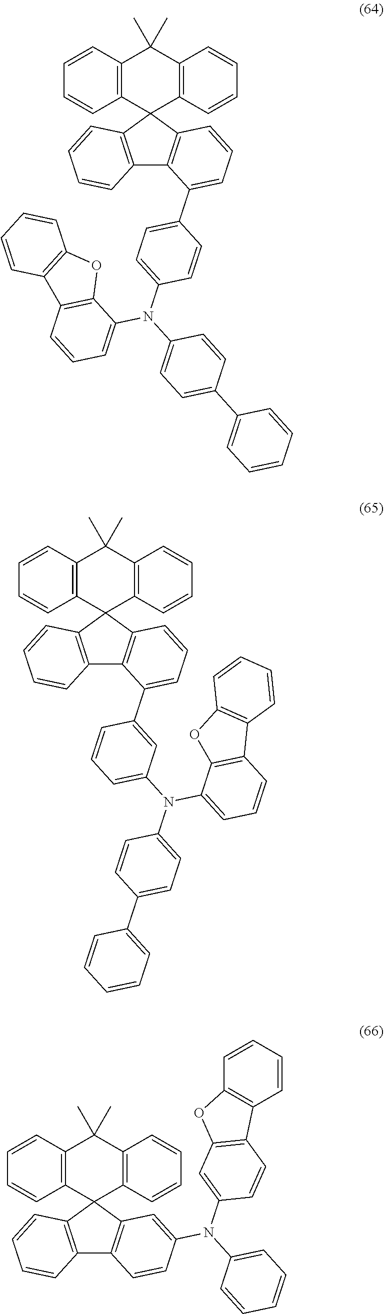 [SMILES: CC1(C)c2ccccc2C2(c3ccccc3-c3c(-c4ccc(N(c5ccc(-c6ccccc6)cc5)c5cccc6c5oc5ccccc56)cc4)cccc32)c2ccccc21 ; CC1(C)c2ccccc2C2(c3ccccc3-c3c(-c4cccc(N(c5ccc(-c6ccccc6)cc5)c5cccc6c5oc5ccccc56)c4)cccc32)c2ccccc21 ; CC1(C)c2ccccc2C2(c3ccccc3-c3ccc(N(c4ccccc4)c4ccc5c(c4)oc4ccccc45)cc32)c2ccccc21]

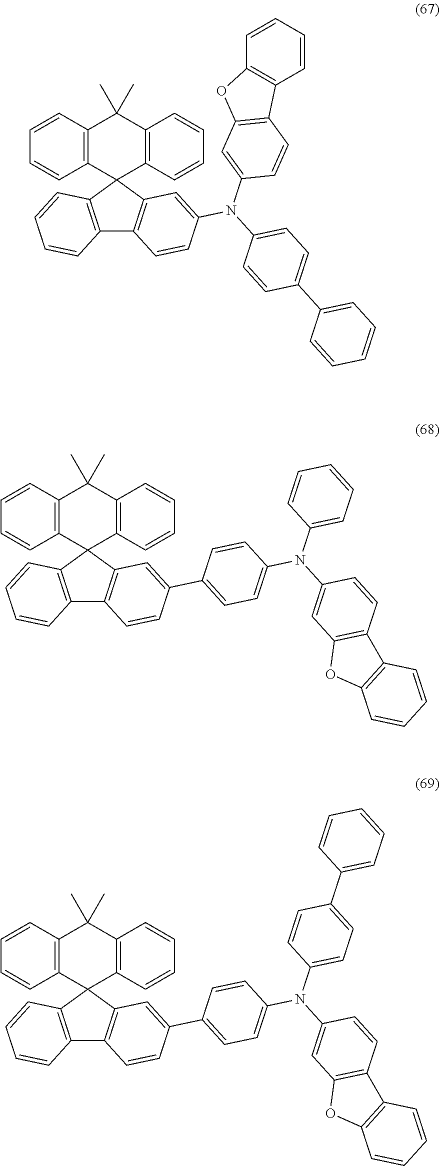 [SMILES: CC1(C)c2ccccc2C2(c3ccccc3-c3ccc(N(c4ccc(-c5ccccc5)cc4)c4ccc5c(c4)oc4ccccc45)cc32)c2ccccc21 ; CC1(C)c2ccccc2C2(c3ccccc3-c3ccc(-c4ccc(N(c5ccccc5)c5ccc6c(c5)oc5ccccc56)cc4)cc32)c2ccccc21 ; CC1(C)c2ccccc2C2(c3ccccc3-c3ccc(-c4ccc(N(c5ccc(-c6ccccc6)cc5)c5ccc6c(c5)oc5ccccc56)cc4)cc32)c2ccccc21]

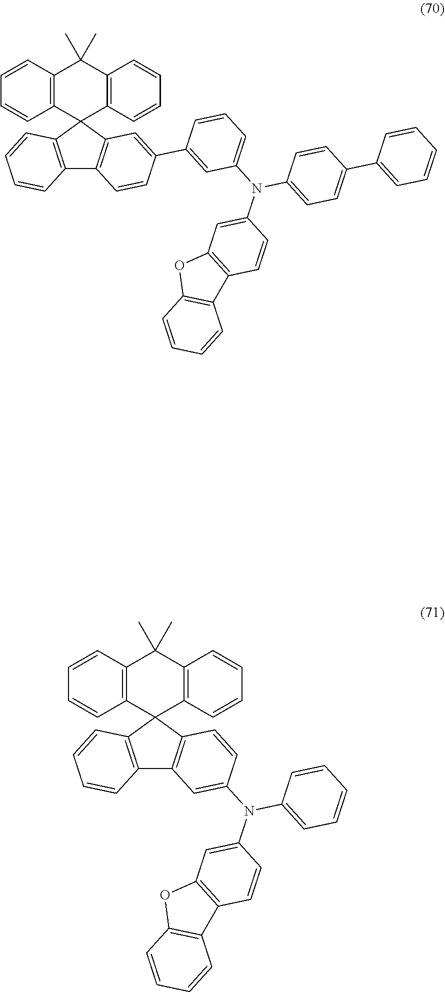 [SMILES: CC1(C)c2ccccc2C2(c3ccccc3-c3ccc(-c4cccc(N(c5ccc(-c6ccccc6)cc5)c5ccc6c(c5)oc5ccccc56)c4)cc32)c2ccccc21 ; CC1(C)c2ccccc2C2(c3ccccc3-c3cc(N(c4ccccc4)c4ccc5c(c4)oc4ccccc45)ccc32)c2ccccc21]

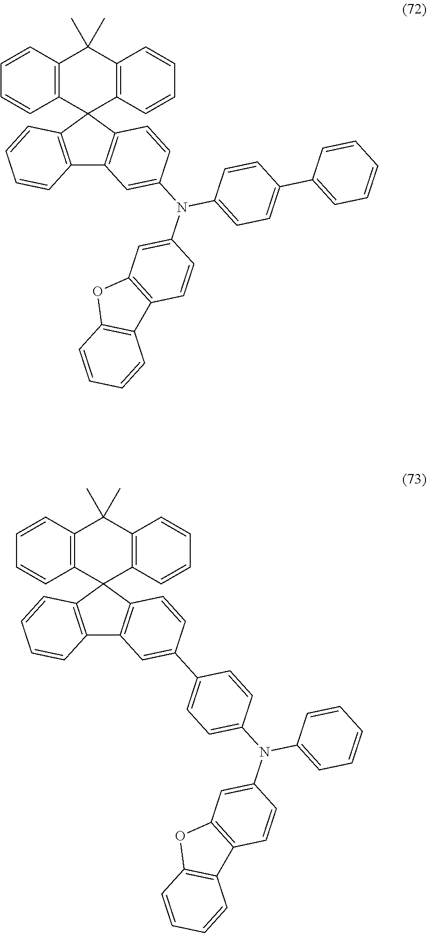 [SMILES: CC1(C)c2ccccc2C2(c3ccccc3-c3cc(N(c4ccc(-c5ccccc5)cc4)c4ccc5c(c4)oc4ccccc45)ccc32)c2ccccc21 ; CC1(C)c2ccccc2C2(c3ccccc3-c3cc(-c4ccc(N(c5ccccc5)c5ccc6c(c5)oc5ccccc56)cc4)ccc32)c2ccccc21]

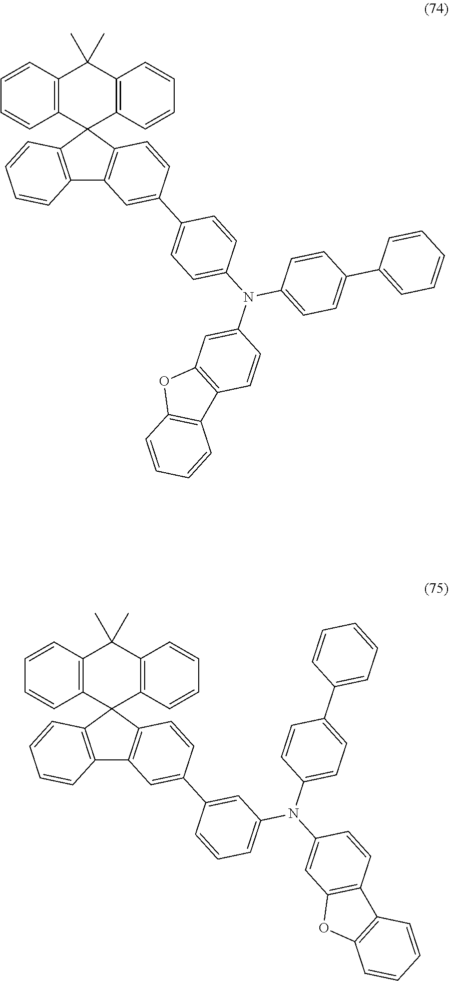 [SMILES: CC1(C)c2ccccc2C2(c3ccccc3-c3cc(-c4ccc(N(c5ccc(-c6ccccc6)cc5)c5ccc6c(c5)oc5ccccc56)cc4)ccc32)c2ccccc21 ; CC1(C)c2ccccc2C2(c3ccccc3-c3cc(-c4cccc(N(c5ccc(-c6ccccc6)cc5)c5ccc6c(c5)oc5ccccc56)c4)ccc32)c2ccccc21]

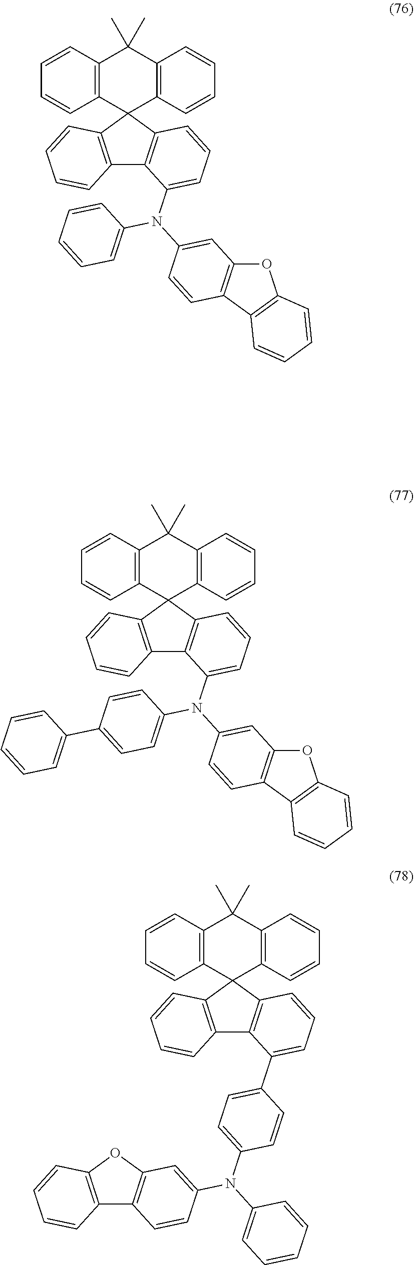 [SMILES: CC1(C)c2ccccc2C2(c3ccccc3-c3c(N(c4ccccc4)c4ccc5c(c4)oc4ccccc45)cccc32)c2ccccc21 ; CC1(C)c2ccccc2C2(c3ccccc3-c3c(N(c4ccc(-c5ccccc5)cc4)c4ccc5c(c4)oc4ccccc45)cccc32)c2ccccc21 ; CC1(C)c2ccccc2C2(c3ccccc3-c3c(-c4ccc(N(c5ccccc5)c5ccc6c(c5)oc5ccccc56)cc4)cccc32)c2ccccc21]

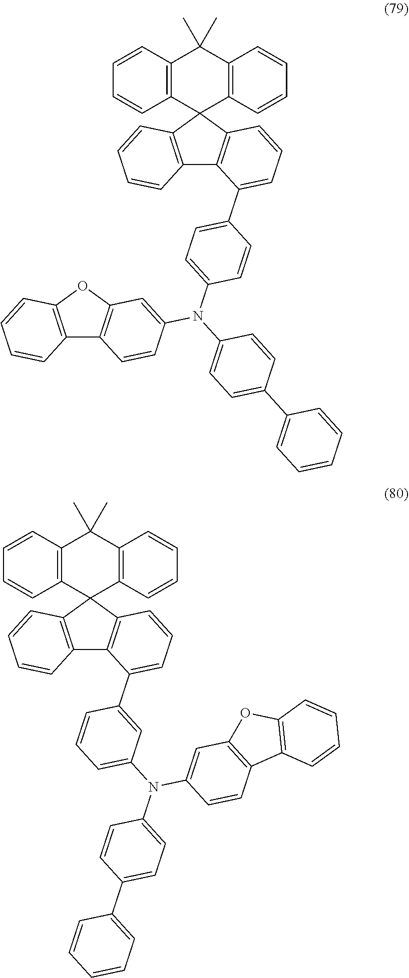 [SMILES: CC1(C)c2ccccc2C2(c3ccccc3-c3c(-c4ccc(N(c5ccc(-c6ccccc6)cc5)c5ccc6c(c5)oc5ccccc56)cc4)cccc32)c2ccccc21 ; CC1(C)c2ccccc2C2(c3ccccc3-c3c(-c4cccc(N(c5ccc(-c6ccccc6)cc5)c5ccc6c(c5)oc5ccccc56)c4)cccc32)c2ccccc21]

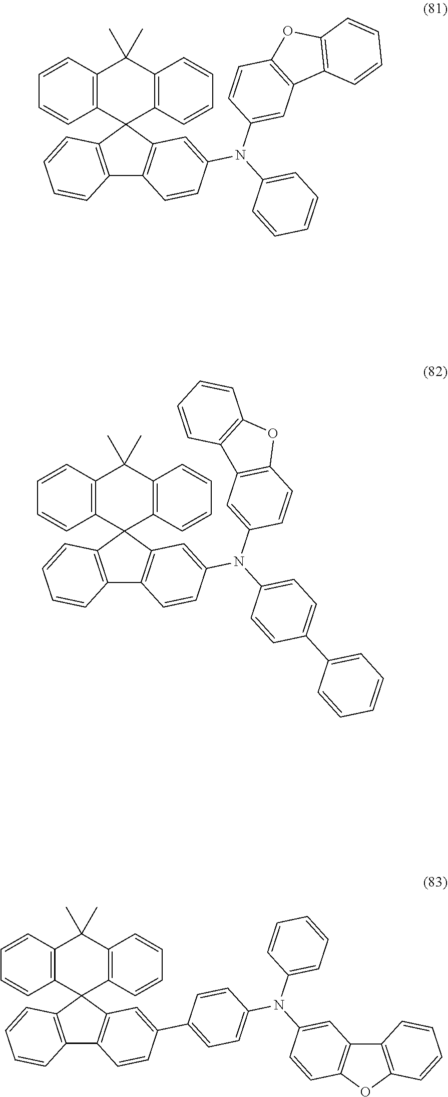 [SMILES: CC1(C)c2ccccc2C2(c3ccccc3-c3ccc(N(c4ccccc4)c4ccc5oc6ccccc6c5c4)cc32)c2ccccc21 ; CC1(C)c2ccccc2C2(c3ccccc3-c3ccc(N(c4ccc(-c5ccccc5)cc4)c4ccc5oc6ccccc6c5c4)cc32)c2ccccc21 ; CC1(C)c2ccccc2C2(c3ccccc3-c3ccc(-c4ccc(N(c5ccccc5)c5ccc6oc7ccccc7c6c5)cc4)cc32)c2ccccc21]

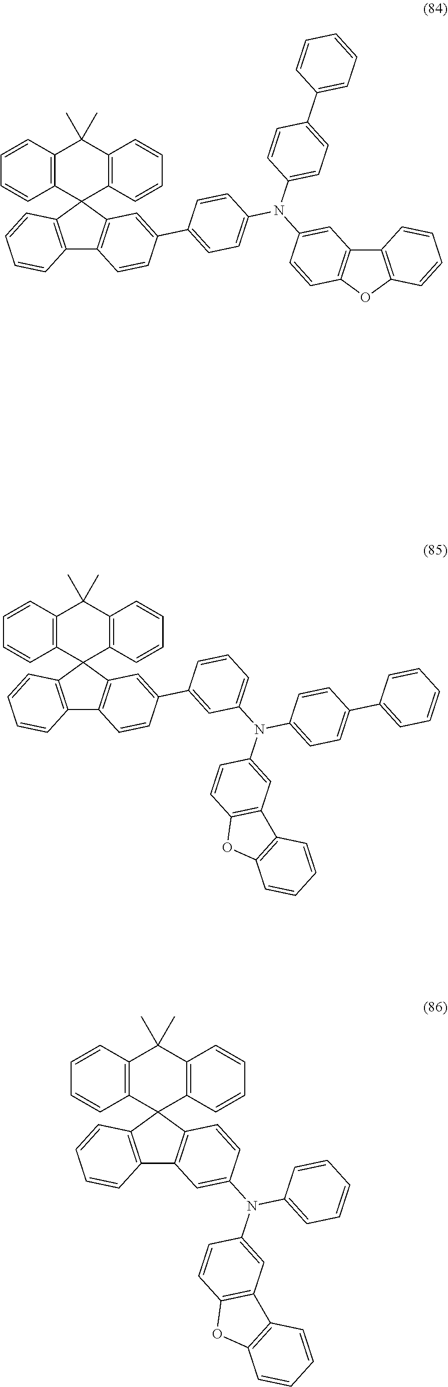 [SMILES: CC1(C)c2ccccc2C2(c3ccccc3-c3ccc(-c4ccc(N(c5ccc(-c6ccccc6)cc5)c5ccc6oc7ccccc7c6c5)cc4)cc32)c2ccccc21 ; CC1(C)c2ccccc2C2(c3ccccc3-c3ccc(-c4cccc(N(c5ccc(-c6ccccc6)cc5)c5ccc6oc7ccccc7c6c5)c4)cc32)c2ccccc21 ; CC1(C)c2ccccc2C2(c3ccccc3-c3cc(N(c4ccccc4)c4ccc5oc6ccccc6c5c4)ccc32)c2ccccc21]

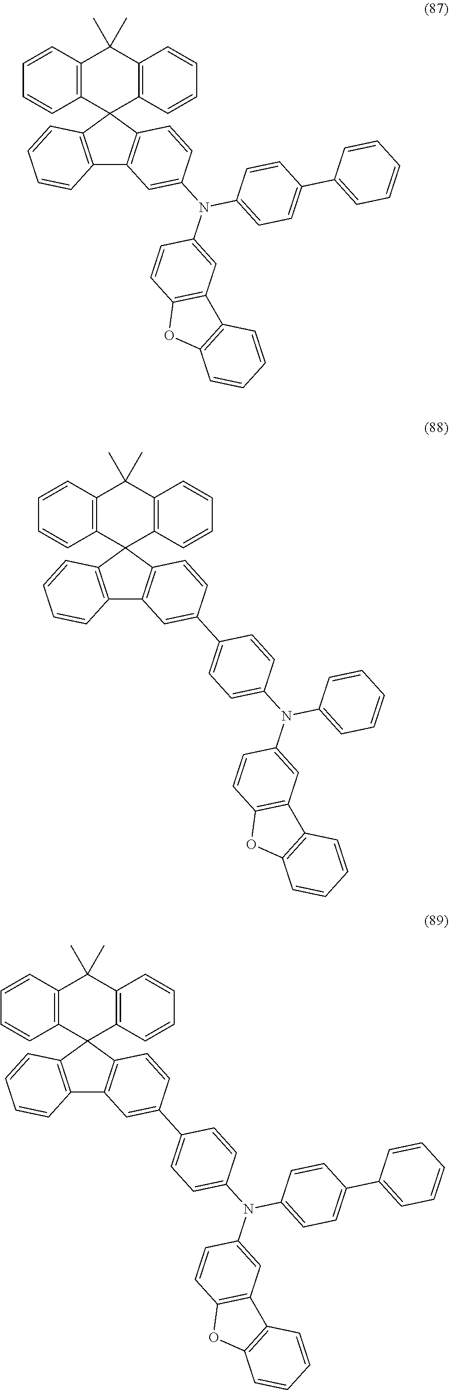 [SMILES: CC1(C)c2ccccc2C2(c3ccccc3-c3cc(N(c4ccc(-c5ccccc5)cc4)c4ccc5oc6ccccc6c5c4)ccc32)c2ccccc21 ; CC1(C)c2ccccc2C2(c3ccccc3-c3cc(-c4ccc(N(c5ccccc5)c5ccc6oc7ccccc7c6c5)cc4)ccc32)c2ccccc21 ; CC1(C)c2ccccc2C2(c3ccccc3-c3cc(-c4ccc(N(c5ccc(-c6ccccc6)cc5)c5ccc6oc7ccccc7c6c5)cc4)ccc32)c2ccccc21]

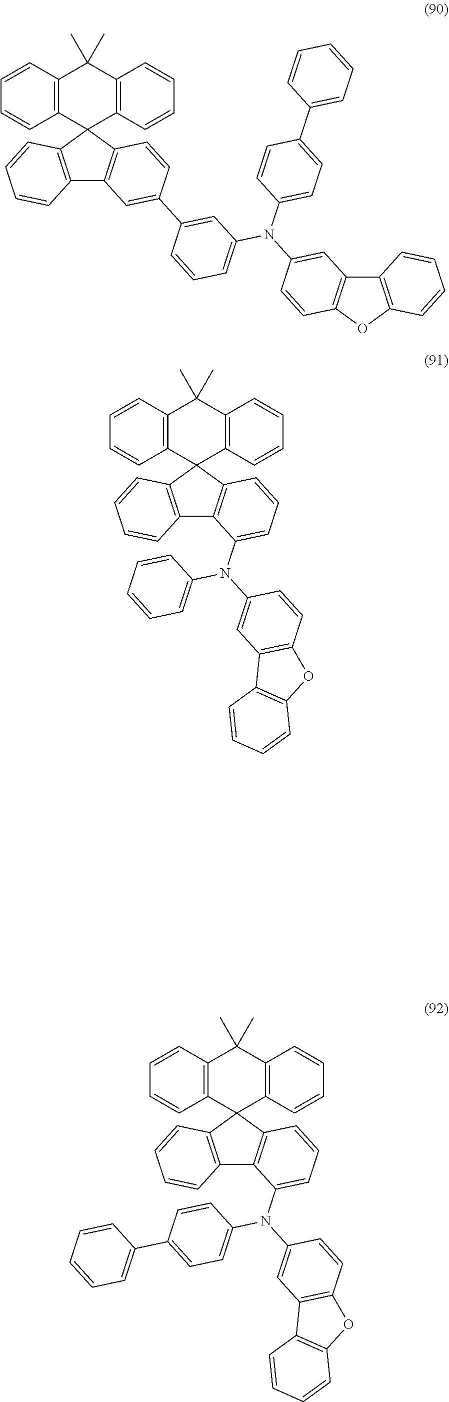 [SMILES: CC1(C)c2ccccc2C2(c3ccccc3-c3cc(-c4cccc(N(c5ccc(-c6ccccc6)cc5)c5ccc6oc7ccccc7c6c5)c4)ccc32)c2ccccc21 ; CC1(C)c2ccccc2C2(c3ccccc3-c3c(N(c4ccccc4)c4ccc5oc6ccccc6c5c4)cccc32)c2ccccc21 ; CC1(C)c2ccccc2C2(c3ccccc3-c3c(N(c4ccc(-c5ccccc5)cc4)c4ccc5oc6ccccc6c5c4)cccc32)c2ccccc21]

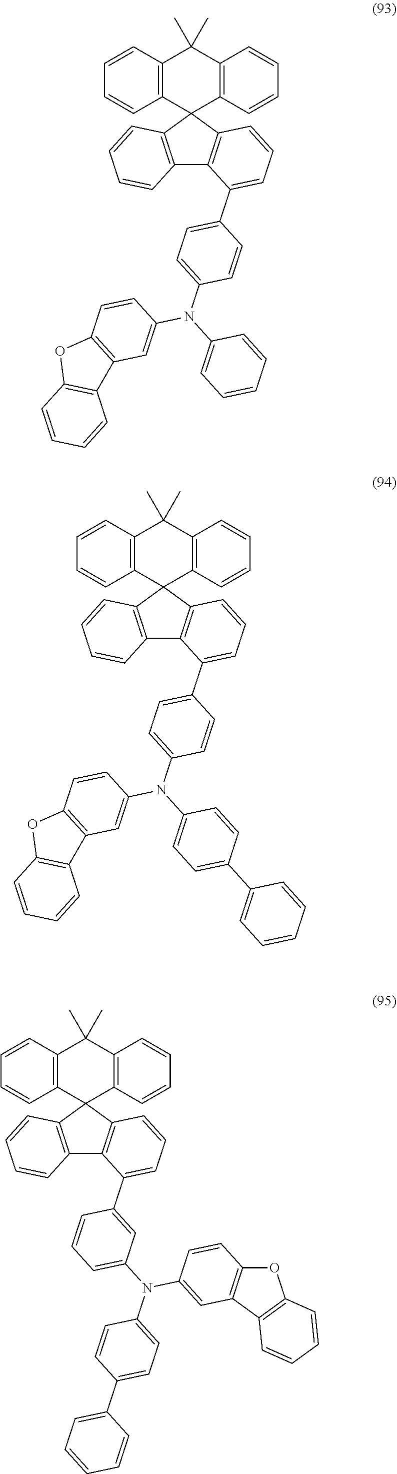 [SMILES: CC1(C)c2ccccc2C2(c3ccccc3-c3c(-c4ccc(N(c5ccccc5)c5ccc6oc7ccccc7c6c5)cc4)cccc32)c2ccccc21 ; CC1(C)c2ccccc2C2(c3ccccc3-c3c(-c4ccc(N(c5ccc(-c6ccccc6)cc5)c5ccc6oc7ccccc7c6c5)cc4)cccc32)c2ccccc21 ; CC1(C)c2ccccc2C2(c3ccccc3-c3c(-c4cccc(N(c5ccc(-c6ccccc6)cc5)c5ccc6oc7ccccc7c6c5)c4)cccc32)c2ccccc21]

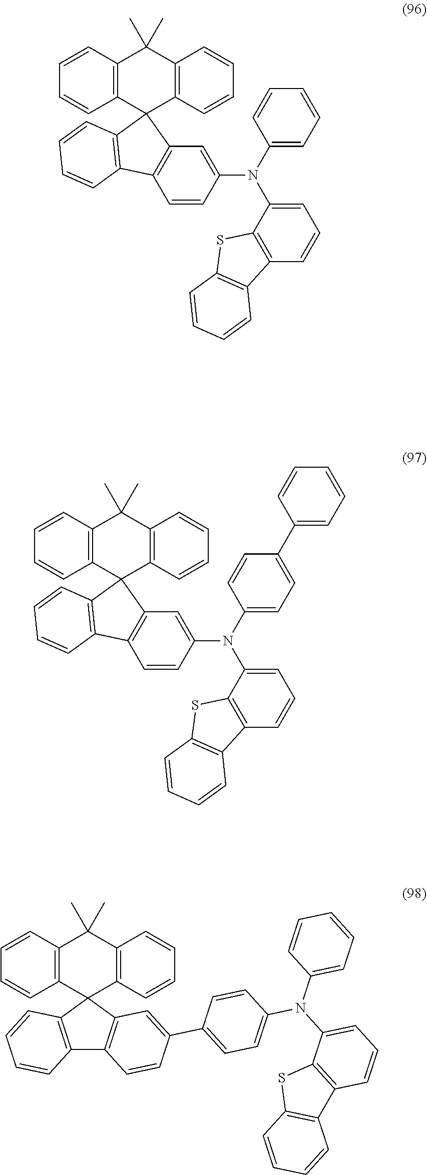 [SMILES: CC1(C)c2ccccc2C2(c3ccccc3-c3ccc(N(c4ccccc4)c4cccc5c4sc4ccccc45)cc32)c2ccccc21 ; CC1(C)c2ccccc2C2(c3ccccc3-c3ccc(N(c4ccc(-c5ccccc5)cc4)c4cccc5c4sc4ccccc45)cc32)c2ccccc21 ; CC1(C)c2ccccc2C2(c3ccccc3-c3ccc(-c4ccc(N(c5ccccc5)c5cccc6c5sc5ccccc56)cc4)cc32)c2ccccc21]

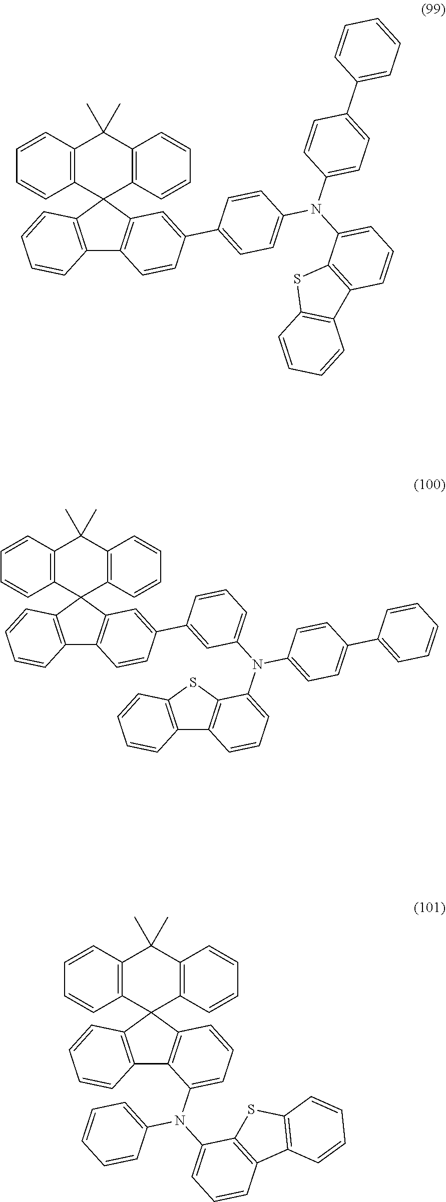 [SMILES: CC1(C)c2ccccc2C2(c3ccccc3-c3ccc(-c4ccc(N(c5ccc(-c6ccccc6)cc5)c5cccc6c5sc5ccccc56)cc4)cc32)c2ccccc21 ; CC1(C)c2ccccc2C2(c3ccccc3-c3ccc(-c4cccc(N(c5ccc(-c6ccccc6)cc5)c5cccc6c5sc5ccccc56)c4)cc32)c2ccccc21 ; CC1(C)c2ccccc2C2(c3ccccc3-c3c(N(c4ccccc4)c4cccc5c4sc4ccccc45)cccc32)c2ccccc21]

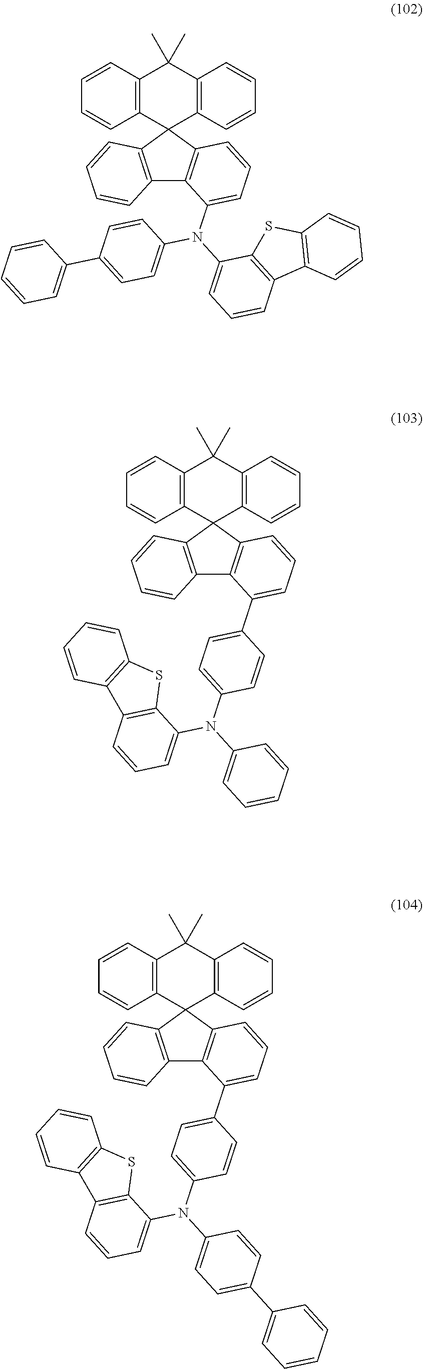 [SMILES: CC1(C)c2ccccc2C2(c3ccccc3-c3c(N(c4ccc(-c5ccccc5)cc4)c4cccc5c4sc4ccccc45)cccc32)c2ccccc21 ; CC1(C)c2ccccc2C2(c3ccccc3-c3c(-c4ccc(N(c5ccccc5)c5cccc6c5sc5ccccc56)cc4)cccc32)c2ccccc21 ; CC1(C)c2ccccc2C2(c3ccccc3-c3c(-c4ccc(N(c5ccc(-c6ccccc6)cc5)c5cccc6c5sc5ccccc56)cc4)cccc32)c2ccccc21]

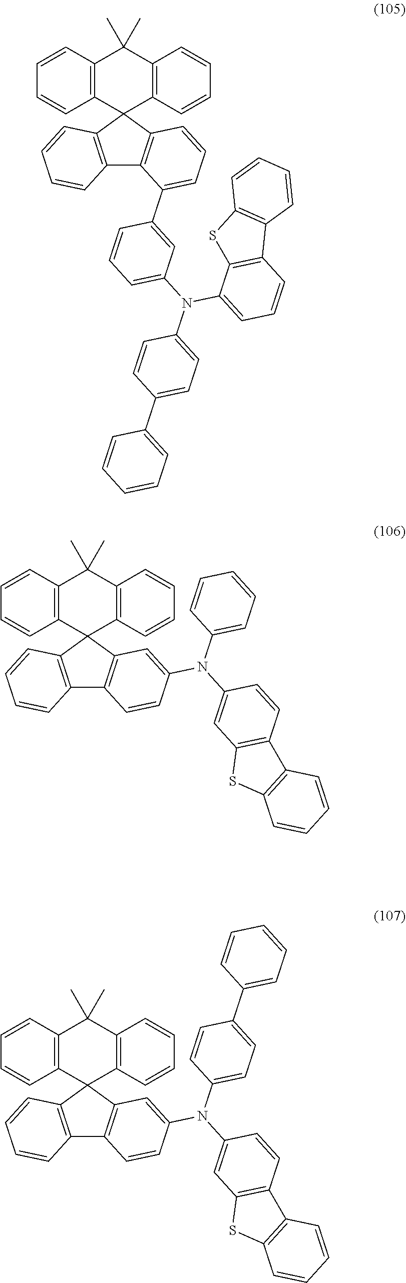 [SMILES: CC1(C)c2ccccc2C2(c3ccccc3-c3c(-c4cccc(N(c5ccc(-c6ccccc6)cc5)c5cccc6c5sc5ccccc56)c4)cccc32)c2ccccc21 ; CC1(C)c2ccccc2C2(c3ccccc3-c3ccc(N(c4ccccc4)c4ccc5c(c4)sc4ccccc45)cc32)c2ccccc21 ; CC1(C)c2ccccc2C2(c3ccccc3-c3ccc(N(c4ccc(-c5ccccc5)cc4)c4ccc5c(c4)sc4ccccc45)cc32)c2ccccc21]

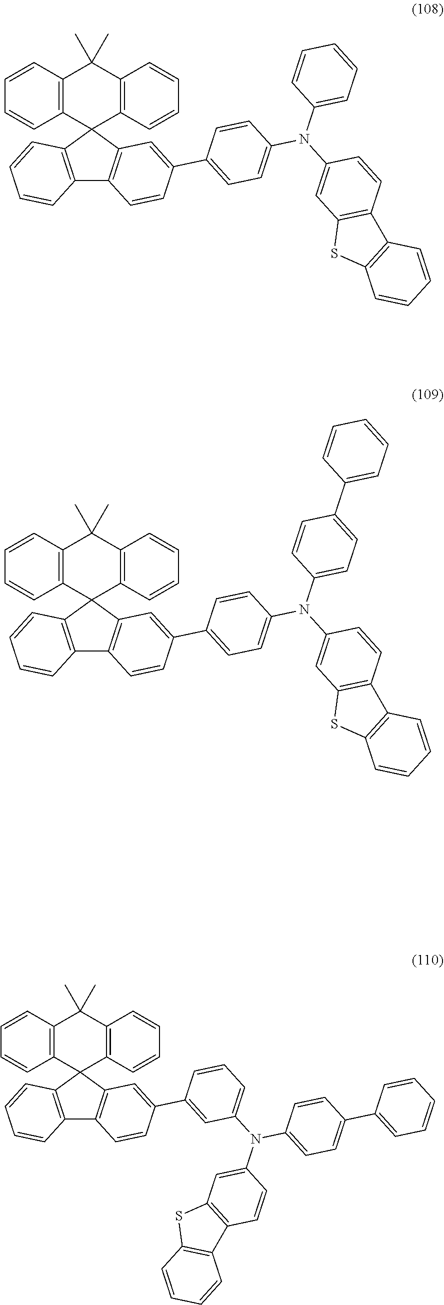 [SMILES: CC1(C)c2ccccc2C2(c3ccccc3-c3ccc(-c4ccc(N(c5ccccc5)c5ccc6c(c5)sc5ccccc56)cc4)cc32)c2ccccc21 ; CC1(C)c2ccccc2C2(c3ccccc3-c3ccc(-c4ccc(N(c5ccc(-c6ccccc6)cc5)c5ccc6c(c5)sc5ccccc56)cc4)cc32)c2ccccc21 ; CC1(C)c2ccccc2C2(c3ccccc3-c3ccc(-c4cccc(N(c5ccc(-c6ccccc6)cc5)c5ccc6c(c5)sc5ccccc56)c4)cc32)c2ccccc21]

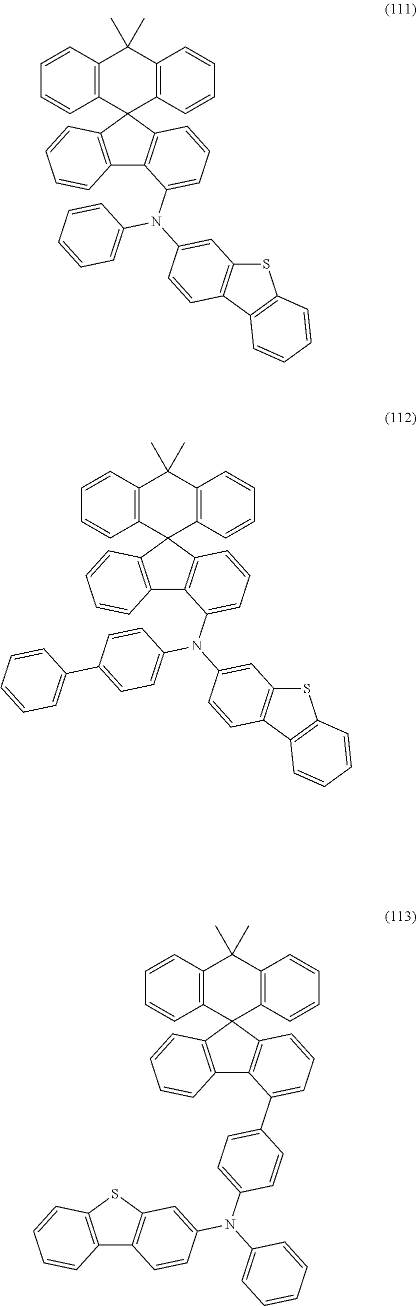 [SMILES: CC1(C)c2ccccc2C2(c3ccccc3-c3c(N(c4ccccc4)c4ccc5c(c4)sc4ccccc45)cccc32)c2ccccc21 ; CC1(C)c2ccccc2C2(c3ccccc3-c3c(N(c4ccc(-c5ccccc5)cc4)c4ccc5c(c4)sc4ccccc45)cccc32)c2ccccc21 ; CC1(C)c2ccccc2C2(c3ccccc3-c3c(-c4ccc(N(c5ccccc5)c5ccc6c(c5)sc5ccccc56)cc4)cccc32)c2ccccc21]

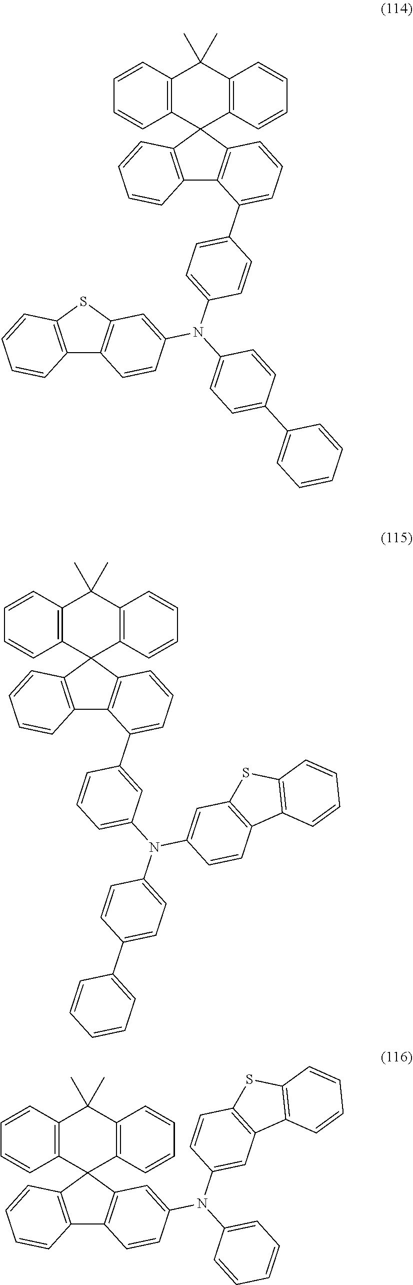

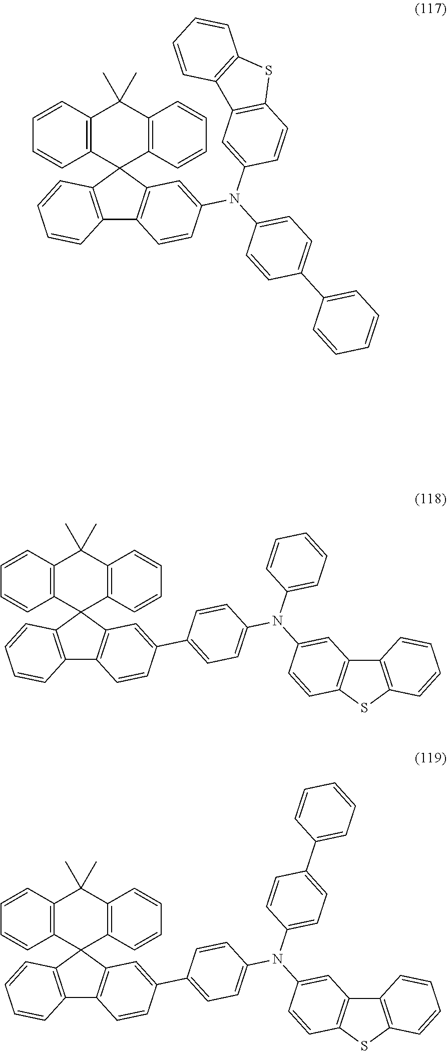 [SMILES: CC1(C)c2ccccc2C2(c3ccccc3-c3ccc(N(c4ccc(-c5ccccc5)cc4)c4ccc5sc6ccccc6c5c4)cc32)c2ccccc21 ; CC1(C)c2ccccc2C2(c3ccccc3-c3ccc(-c4ccc(N(c5ccccc5)c5ccc6sc7ccccc7c6c5)cc4)cc32)c2ccccc21 ; CC1(C)c2ccccc2C2(c3ccccc3-c3ccc(-c4ccc(N(c5ccc(-c6ccccc6)cc5)c5ccc6sc7ccccc7c6c5)cc4)cc32)c2ccccc21]

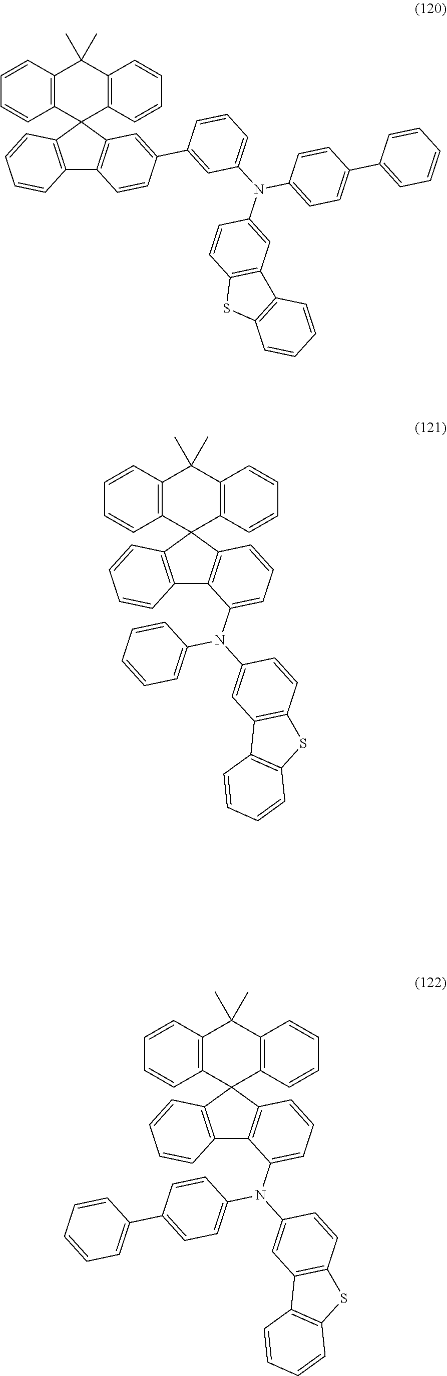 [SMILES: CC1(C)c2ccccc2C2(c3ccccc3-c3ccc(-c4cccc(N(c5ccc(-c6ccccc6)cc5)c5ccc6sc7ccccc7c6c5)c4)cc32)c2ccccc21 ; CC1(C)c2ccccc2C2(c3ccccc3-c3c(N(c4ccccc4)c4ccc5sc6ccccc6c5c4)cccc32)c2ccccc21 ; CC1(C)c2ccccc2C2(c3ccccc3-c3c(N(c4ccc(-c5ccccc5)cc4)c4ccc5sc6ccccc6c5c4)cccc32)c2ccccc21]

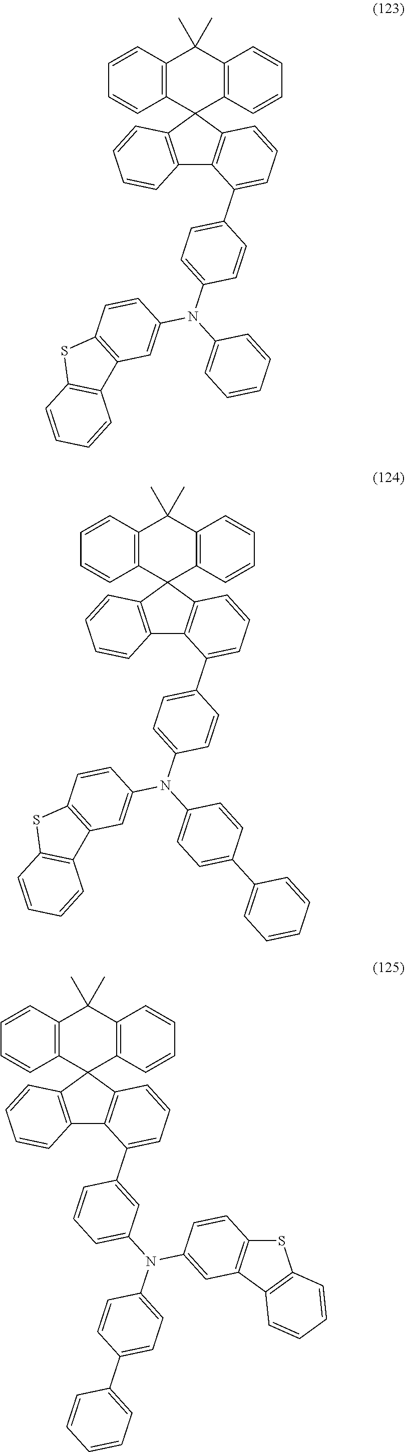 [SMILES: CC1(C)c2ccccc2C2(c3ccccc3-c3c(-c4ccc(N(c5ccccc5)c5ccc6sc7ccccc7c6c5)cc4)cccc32)c2ccccc21 ; CC1(C)c2ccccc2C2(c3ccccc3-c3c(-c4ccc(N(c5ccc(-c6ccccc6)cc5)c5ccc6sc7ccccc7c6c5)cc4)cccc32)c2ccccc21 ; CC1(C)c2ccccc2C2(c3ccccc3-c3c(-c4cccc(N(c5ccc(-c6ccccc6)cc5)c5ccc6sc7ccccc7c6c5)c4)cccc32)c2ccccc21]

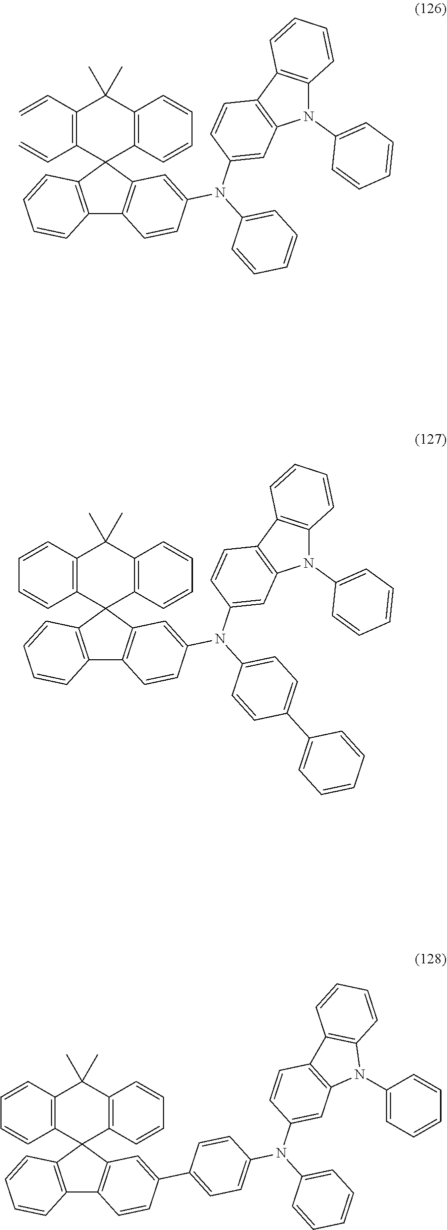 [SMILES: C=CC1=C(C=C)C2(c3ccccc3-c3ccc(N(c4ccccc4)c4ccc5c6ccccc6n(-c6ccccc6)c5c4)cc32)c2ccccc2C1(C)C ; CC1(C)c2ccccc2C2(c3ccccc3-c3ccc(N(c4ccc(-c5ccccc5)cc4)c4ccc5c6ccccc6n(-c6ccccc6)c5c4)cc32)c2ccccc21 ; CC1(C)c2ccccc2C2(c3ccccc3-c3ccc(-c4ccc(N(c5ccccc5)c5ccc6c7ccccc7n(-c7ccccc7)c6c5)cc4)cc32)c2ccccc21]

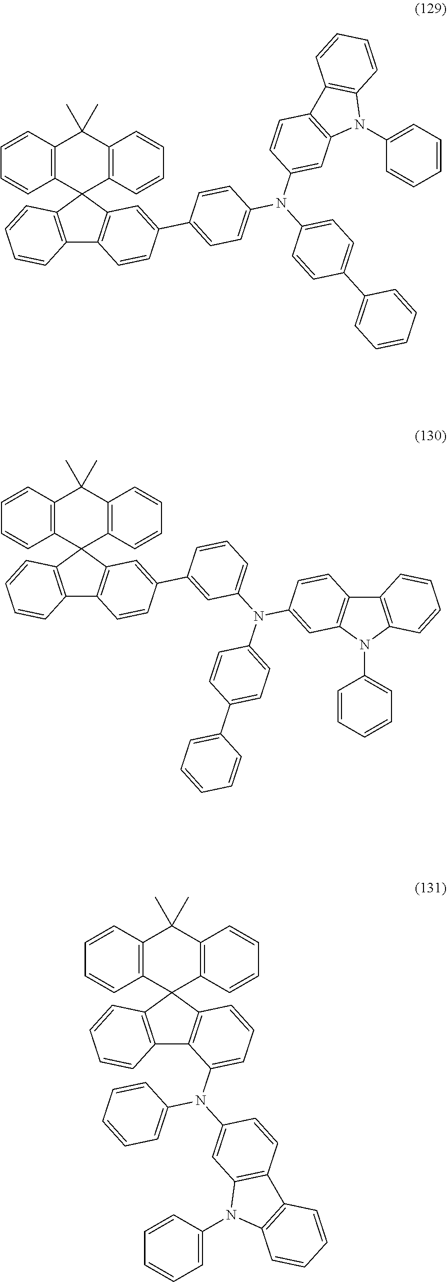 [SMILES: CC1(C)c2ccccc2C2(c3ccccc3-c3ccc(-c4ccc(N(c5ccc(-c6ccccc6)cc5)c5ccc6c7ccccc7n(-c7ccccc7)c6c5)cc4)cc32)c2ccccc21 ; CC1(C)c2ccccc2C2(c3ccccc3-c3ccc(-c4cccc(N(c5ccc(-c6ccccc6)cc5)c5ccc6c7ccccc7n(-c7ccccc7)c6c5)c4)cc32)c2ccccc21 ; CC1(C)c2ccccc2C2(c3ccccc3-c3c(N(c4ccccc4)c4ccc5c6ccccc6n(-c6ccccc6)c5c4)cccc32)c2ccccc21]

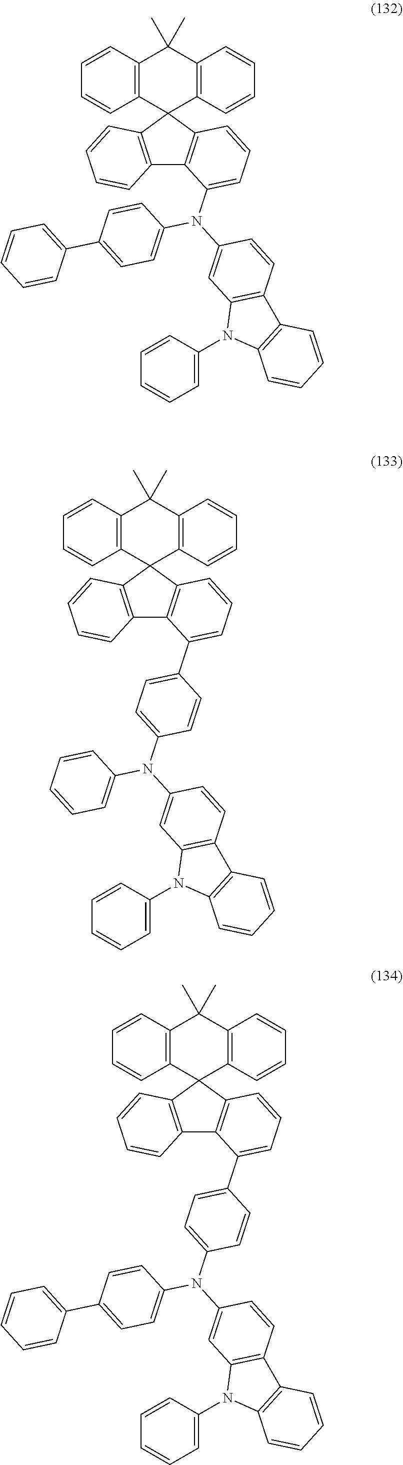 [SMILES: CC1(C)c2ccccc2C2(c3ccccc3-c3c(N(c4ccc(-c5ccccc5)cc4)c4ccc5c6ccccc6n(-c6ccccc6)c5c4)cccc32)c2ccccc21 ; CC1(C)c2ccccc2C2(c3ccccc3-c3c(-c4ccc(N(c5ccccc5)c5ccc6c7ccccc7n(-c7ccccc7)c6c5)cc4)cccc32)c2ccccc21 ; CC1(C)c2ccccc2C2(c3ccccc3-c3c(-c4ccc(N(c5ccc(-c6ccccc6)cc5)c5ccc6c7ccccc7n(-c7ccccc7)c6c5)cc4)cccc32)c2ccccc21]

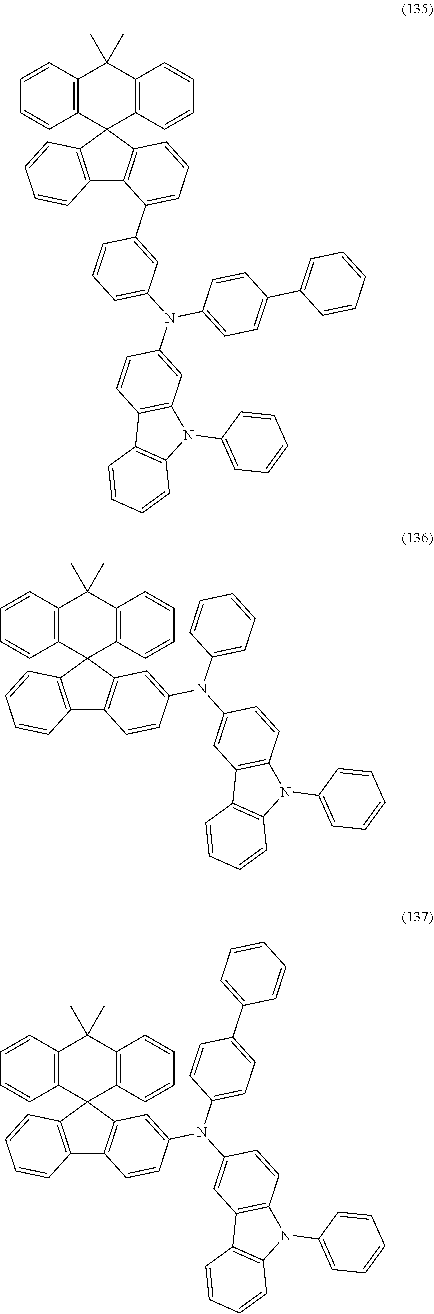 [SMILES: CC1(C)c2ccccc2C2(c3ccccc3-c3c(-c4cccc(N(c5ccc(-c6ccccc6)cc5)c5ccc6c7ccccc7n(-c7ccccc7)c6c5)c4)cccc32)c2ccccc21 ; CC1(C)c2ccccc2C2(c3ccccc3-c3ccc(N(c4ccccc4)c4ccc5c(c4)c4ccccc4n5-c4ccccc4)cc32)c2ccccc21 ; CC1(C)c2ccccc2C2(c3ccccc3-c3ccc(N(c4ccc(-c5ccccc5)cc4)c4ccc5c(c4)c4ccccc4n5-c4ccccc4)cc32)c2ccccc21]

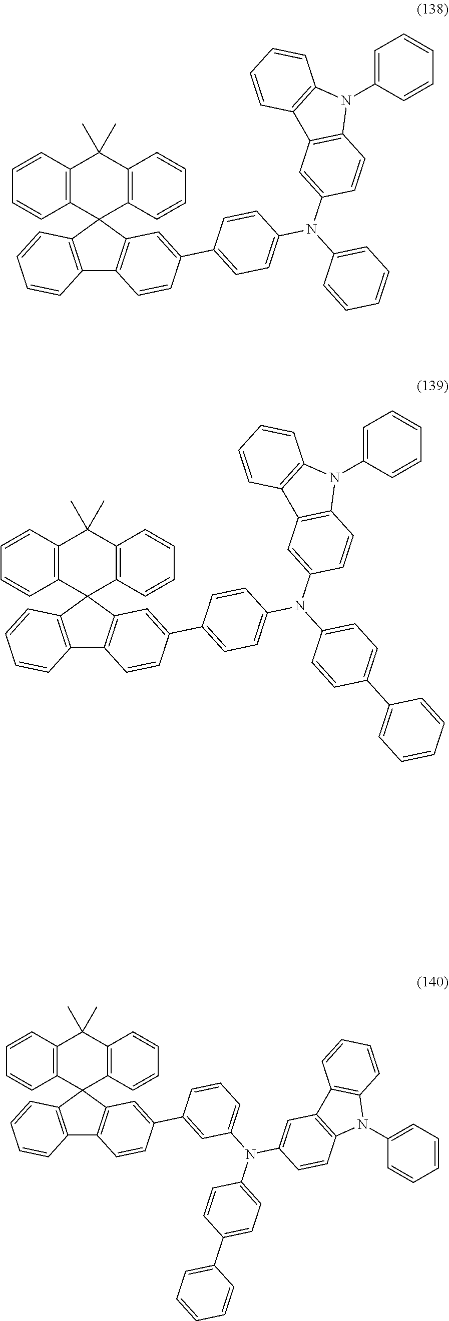 [SMILES: CC1(C)c2ccccc2C2(c3ccccc3-c3ccc(-c4ccc(N(c5ccccc5)c5ccc6c(c5)c5ccccc5n6-c5ccccc5)cc4)cc32)c2ccccc21 ; CC1(C)c2ccccc2C2(c3ccccc3-c3ccc(-c4ccc(N(c5ccc(-c6ccccc6)cc5)c5ccc6c(c5)c5ccccc5n6-c5ccccc5)cc4)cc32)c2ccccc21 ; CC1(C)c2ccccc2C2(c3ccccc3-c3ccc(-c4cccc(N(c5ccc(-c6ccccc6)cc5)c5ccc6c(c5)c5ccccc5n6-c5ccccc5)c4)cc32)c2ccccc21]

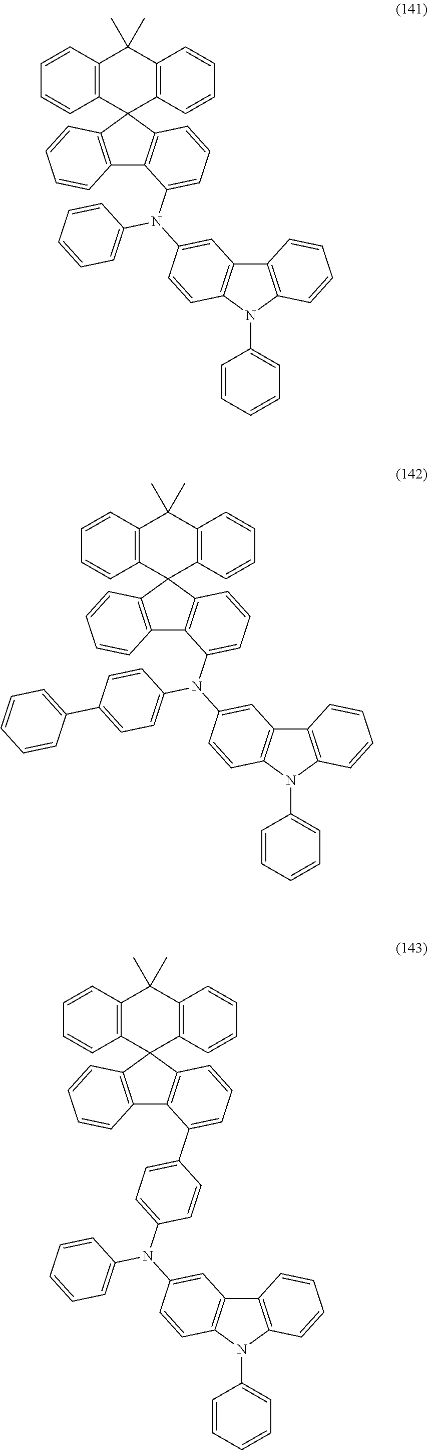 [SMILES: CC1(C)c2ccccc2C2(c3ccccc3-c3c(N(c4ccccc4)c4ccc5c(c4)c4ccccc4n5-c4ccccc4)cccc32)c2ccccc21 ; CC1(C)c2ccccc2C2(c3ccccc3-c3c(N(c4ccc(-c5ccccc5)cc4)c4ccc5c(c4)c4ccccc4n5-c4ccccc4)cccc32)c2ccccc21 ; CC1(C)c2ccccc2C2(c3ccccc3-c3c(-c4ccc(N(c5ccccc5)c5ccc6c(c5)c5ccccc5n6-c5ccccc5)cc4)cccc32)c2ccccc21]

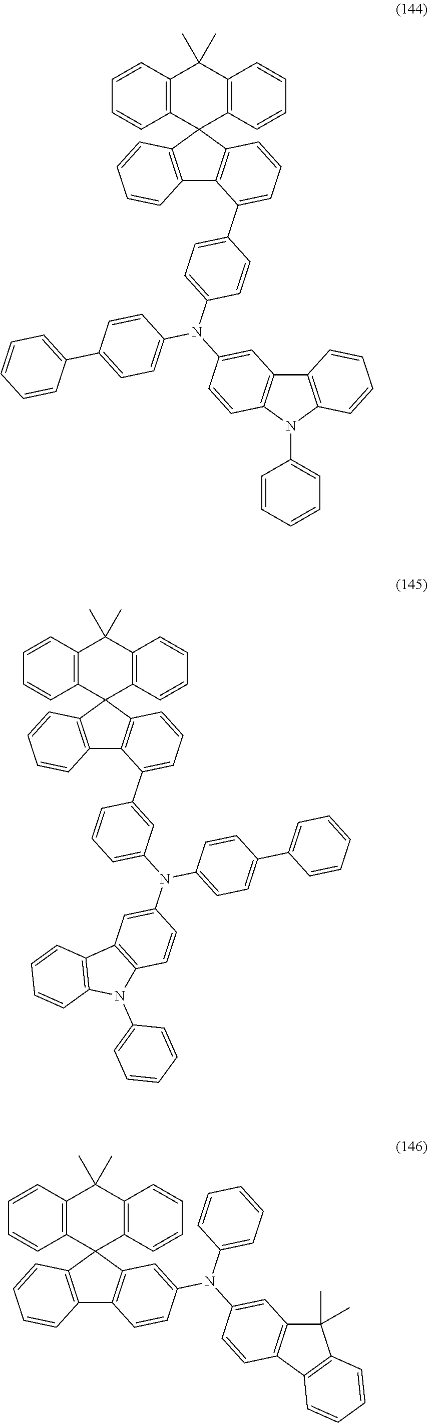 [SMILES: CC1(C)c2ccccc2C2(c3ccccc3-c3c(-c4ccc(N(c5ccc(-c6ccccc6)cc5)c5ccc6c(c5)c5ccccc5n6-c5ccccc5)cc4)cccc32)c2ccccc21 ; CC1(C)c2ccccc2C2(c3ccccc3-c3c(-c4cccc(N(c5ccc(-c6ccccc6)cc5)c5ccc6c(c5)c5ccccc5n6-c5ccccc5)c4)cccc32)c2ccccc21 ; CC1(C)c2ccccc2-c2ccc(N(c3ccccc3)c3ccc4c(c3)C3(c5ccccc5-4)c4ccccc4C(C)(C)c4ccccc43)cc21]

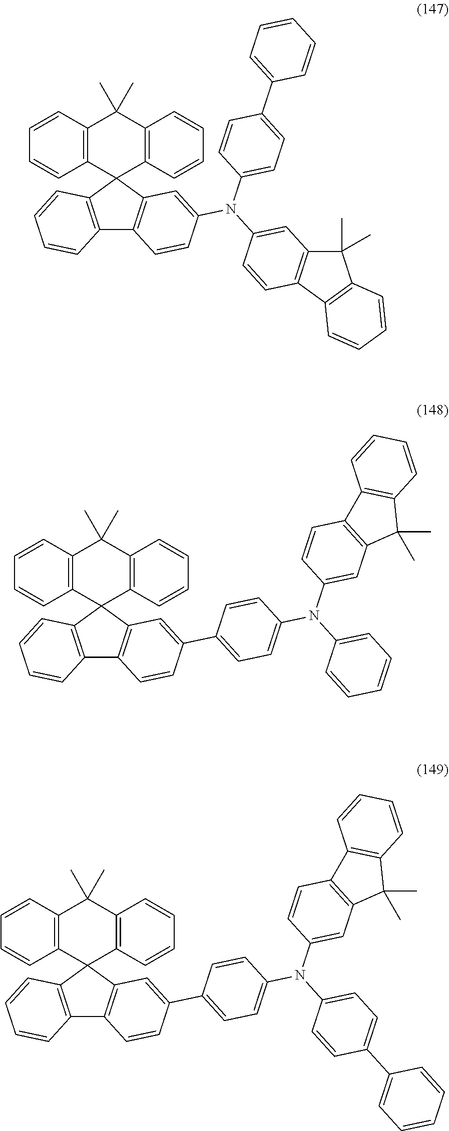 [SMILES: CC1(C)c2ccccc2-c2ccc(N(c3ccc(-c4ccccc4)cc3)c3ccc4c(c3)C3(c5ccccc5-4)c4ccccc4C(C)(C)c4ccccc43)cc21 ; CC1(C)c2ccccc2-c2ccc(N(c3ccccc3)c3ccc(-c4ccc5c(c4)C4(c6ccccc6-5)c5ccccc5C(C)(C)c5ccccc54)cc3)cc21 ; CC1(C)c2ccccc2-c2ccc(N(c3ccc(-c4ccccc4)cc3)c3ccc(-c4ccc5c(c4)C4(c6ccccc6-5)c5ccccc5C(C)(C)c5ccccc54)cc3)cc21]

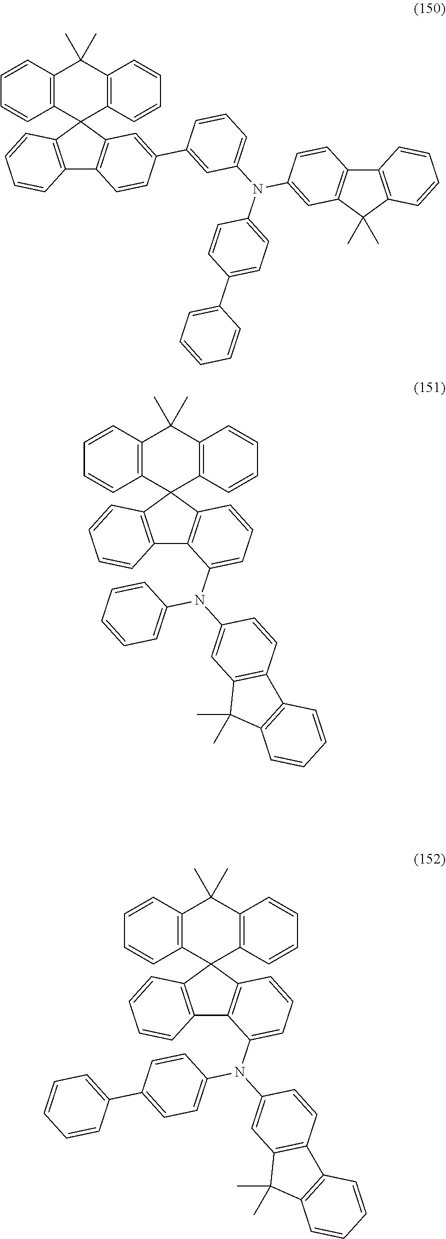

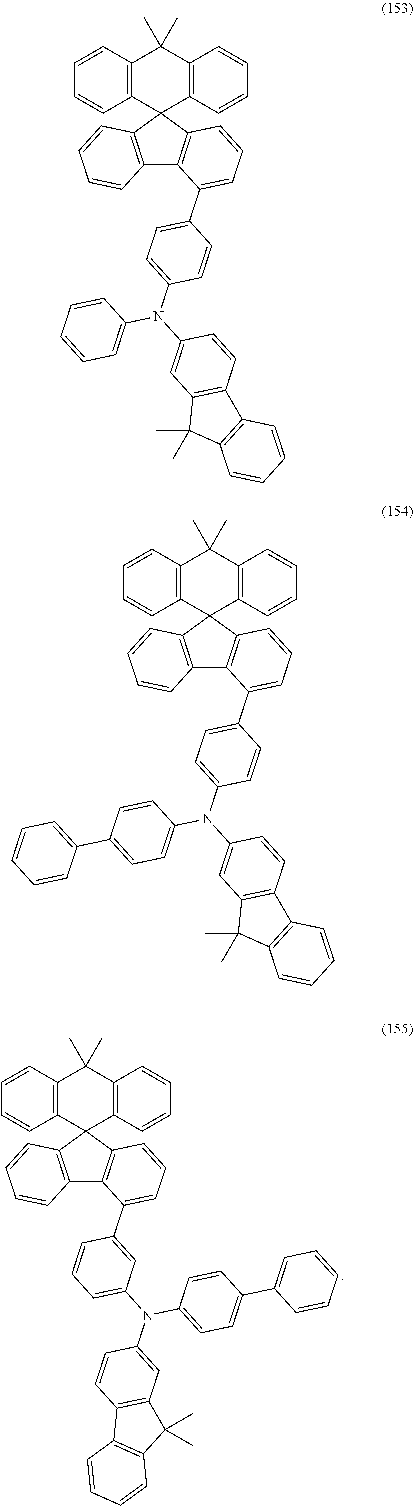

9. An organic electroluminescent device comprising:
an anode;
a cathode; and
one or more organic material layers provided between the anode and the cathode,
wherein at least one of the one or more organic material layers includes the compound represented by Chemical Formula 1 of claim 1.

10. The organic electroluminescent device of claim 9, wherein the organic material layer includes one or more layers selected from the group consisting of a hole injection layer, a hole transport layer, a hole transport auxiliary layer, an electron transport layer, an electron transport auxiliary layer and a light emitting layer.
